SEQUENCE LISTING (67 sequences)

SEQ ID NO: 1 - DNA, 1675 bp, Homo sapiens
  source 1..1675: mol_type = other DNA
attcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctcttatatctcactcccactattacccctttattttcaaacagggaaacagtcttcaagttccacttggtaaaaaatgtgaaccccttgtatatagagtttggctcacagtgtaaagggcctcagtgattcacattttccagattaggaatctgatgctcaaagaagttaaatggcatagttggggtgacacagctgtctagtgggaggccagccttctatattttagccagcgttctttcctgcgggccaggtcatgaggagtatgcagactctaagagggagcaaaagtatctgaaggatttaatattttagcaaggaatagatatacaatcatcccttggtctccctgggggattggtttcaggaccccttcttggacaccaaatctatggatatttaagtcccttctataaaatggtatagtatttgcatataacctatccacatcctcctgtatactttaaatcatttctagattacttgtaatacctaatacaatgtaaatgctatgcaaatagttgttattgtttaaggaataatgacaagaaaaaaaagtctgtacatgctcagtaaagacacaaccatccctttttttccccagtgtttttgatccatggtttgctgaatccacagatgtggagcccctggatacggaaggcccgctgtactttgaatgacaaataacagatttaaaattttcaaggcatagttttatacctga

SEQ ID NO: 2 - DNA, 1675 bp, Homo sapiens; identical to SEQ ID NO: 1
  source 1..1675: mol_type = other DNA

SEQ ID NO: 3 - DNA, 1508 bp, Homo sapiens
  source 1..1508: mol_type = other DNA
ttttgaaacccttcaaaggcagagacttgtccagcctaacctgcctgctgctcctagctcctgaggctcagggcccttggcttctgtccgctctgctcagggccctccagcgtggccactgctcagccatgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtgatctcgaaccgaacagcagtgcttccaagataatctttggatcagggaccagactcagcatccggccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccctcgctccttcctctgcattgcccctcttctccctctccaaacagagggaactctcctacccccaaggaggtgaaagctgctaccacctctgtgcccccccggtaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctggagaatgttgtgaggggtttatttttttttaatagtgttcataaagaaatacatagtattcttcttctcaagacgtggggggaaattatctcattatcgaggccctgctatgctgtgtgtctgggcgtgttgtatgtcctgctgccgatgccttcattaaaatgatttggaa

SEQ ID NO: 4 - DNA, 1221 bp, Homo sapiens
  source 1..1221: mol_type = other DNA
gggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagccggacagggggcgtaaagaattcacccctccactttgggaacgggaccaggctcactgtgacagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaaggcagccctggaagtggagttaggagcttctaacccgtcatggtttcaatacacattcttcttttgccagcgcttctgaagagctgctctcacctctctgcatcccaatagatatccccctatgtgcatgcacacctgcacactcacggctgaaatctccctaacccagggggaccttagcatgcctaagtgactaaaccaataaaaatgttctggtctggcctgaaaaaaaaaaaaaaaaaaaaaaaaaaaaaaaaaaaaaaaaaaaaaaaaaaaaaaaaaaaaa

SEQ ID NO: 5 - DNA, 1434 bp, Homo sapiens
  source 1..1434: mol_type = other DNA
gatggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgcgggcgccgtggatagagcaggaggggccggagtattgggaccaggagacacggaatatgaaggcccactcacagactgaccgagcgaacctggggaccctgcgcggctactacaaccagagcgaggacggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagagcaccccacccccatgtccaccatgaccctcttcccacgctgacctgtgctccctctccaatcatctttcctgttccagagaggtggggctgaggtgtctccatctctgtctcaacttcatggtgcactgagctgtaacttcttccttccctattaaaattagaacctgagtataaaaaaaaaaaaaaaaaaaa

SEQ ID NO: 6 - DNA, 4427 bp, Homo sapiens
  source 1..4427: mol_type = other DNA
gtcagcgccccccgcccggcgtctcccggggccaggtccaccctctgctgcgccacctggggcatcctccttccccgttgccagtctcgatccgccccgtcgttcctggccctgggctttgccaccctatgctgacaccccgtcccagtcccccttaccattccccttcgaccaccccacttccgaattggagccgcttcaactggccctgggcttagccactctgtgctgaccactctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctagttttagcactgaaaccctcagtcctaggaaaacagggatggttggtcactgtctctgggtgactcttgattcccggccagtttctccacctggggctgtgtttctcgtcctgcatccttctccaggcaggtccccaagcatcgcccccctgctgtggctgttcccaagttcttagggtaccccacgtgggtttatcaaccacttggtgaggctggtaccctgcccccattcctgcaccccaattgccttagtggctagggggttgggggctagagtaggaggggctggagccaggattcttagggctgaacagagaagagctgggggcctgggctcctgggtttgagagaggaggggctggggcctggactcctgggtccgagggaggaggggctggggcctggactcctgggtctgagggtggagggactgggggcctggactcctgggtccgagggaggaggggctggggcctggactcgtgggtctgagggaggaggggctgggggcctggacttctgggtcttagggaggcggggctgggcctggacccctgggtctgaatggggagaggctgggggcctggactccttcatctgagggcggaagggctggggcctggcctcctgggttgaatggggaggggttgggcctggactctggagtccctggtgcccaggcctcaggcatctttcacagggatgcctgtactgggcaggtccttgaaagggaaaggcccattgctctccttgcccccctcccctatcgccatgacaactgggtggaaataaacgagccgagttcatcccgttcccagggcacgtgcggccccttcacagcccgagtttccatgacctcatgctcttggccctcgtagctccctcccgcctcctccagatgggcagctttggagaggtgagggacttggggggtaatttatcccgtggatctaggagtttagcttcactccttcctcagctccagttcaggtcccggagcccacccagtgtccacaaggcctggggcaagtccctcctccgaccccctggacttcggcttttgtccccccaagttttggacccctaagggaagaatgagaaacggtggcccgtgtcagcccctggctgcagggccccgtgcagagggggcctcagtgaactggagtgtgacagcctggggcccaggcacacaggtgtgcagctgtctcacccctctgggagtcccgcccaggcccctgagtctgtcccagcacagggtggccttcctccaccctgcatagccctgggcccacggcttcgttcctgcagagtatctgctggggtggtttccgagcttgacccttggaaggacctggctgggtttaaggcaggaggggctgggggccaggactcctggctctgaaggaggaggggctggaacctcttccctagtctgagcactggaagcgccacctgtgggtggtgacgggggttttgccgtgtctaacaggtaccatgtggggttcccgcacccagatgagaagccccctcccttccccgttcacttcctgtttgcagatagccaggagtcctttcgtggtttccactgagcactgaaggcctggccggcctgaccactgggcaaccaggcgtatcttaaacagccagtggccagaggctgttgggtcattttccccactgtcctagcaccgtgtccctggatctgttttcgtggctccctctggagtcccgacttgctgggacaccgtggctggggtaggtgcggctgacggctgtttcccacccccag

SEQ ID NO: 7 - DNA, 1707 bp, synthetic construct
  misc_feature 1..1707: note = Description of Artificial Sequence: Synthetic polynucleotide
  source 1..1707: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 8 - protein, 93 aa, synthetic construct
  REGION 1..93: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIA

SEQ ID NO: 9 - protein, 83 aa, synthetic construct
  REGION 1..83: note = Description of Artificial Sequence: Synthetic polypeptide
DATFGDTCSTHPLKEEIIKKRSETFKAKMLKLGPDGRKALYSKPGSKNGRWNPETHKFCKCGVRIQTSAYTCSKCRNGGSGGS

SEQ ID NO: 10 - protein, 83 aa, synthetic construct
  REGION 1..83: note = Description of Artificial Sequence: Synthetic polypeptide
DATFGDTCSTHPLKEEIIKKRSETVKAKMLKLGPDGRKALYSRPGSKSGRWNPETHKFCKCGVRIQTSAYTCSKCRNGGSGGS

SEQ ID NO: 11 - protein, 83 aa, synthetic construct
  REGION 1..83: note = Description of Artificial Sequence: Synthetic polypeptide
DATFGDTCSTHPLKEEIIKKRSETFKAKMLKLGPDGRKALYSRPGSKSGRWNPETHKFCKCGVRIQTSAYTCSKCRNGGSGGS

SEQ ID NO: 12 - protein, 86 aa, synthetic construct
  REGION 1..86: note = Description of Artificial Sequence: Synthetic polypeptide
DATFGDTCSTHPLKEEIIKKRSETVKAKMLKLGPDGRKALYSRPGSKSGRWNPETHKFCKCGVRIQTSAYTCSKCRNGGSGGTGGS

SEQ ID NO: 13 - protein, 87 aa, synthetic construct
  REGION 1..87: note = Description of Artificial Sequence: Synthetic polypeptide
DATFGDTCSTHPLKEEIIKKRSETVKAKMLKLGPDGRKALYSRPGSKSGRWNPETHKFCKCGVRIQTSAYTCSKCRNGGGGSGGGGS

SEQ ID NO: 14 - protein, 95 aa, synthetic construct
  REGION 1..95: note = Description of Artificial Sequence: Synthetic polypeptide
DATFGDTCSTHPLKEEIIKKRSETVKAKMLKLGPDGRKALYSRPGSKSGRWNPETHKFCKCGVRIQTSAYTCSKCRNKESGSVSSEQLAQFRSLD

SEQ ID NO: 15 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 16 - protein, 1053 aa, synthetic construct
  REGION 1..1053: note = Description of Artificial Sequence: Synthetic polypeptide
MKRNYILGLEIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 17 - RNA, 21 nt, synthetic construct
  misc_feature 1..21: note = Description of Artificial Sequence: Synthetic oligonucleotide
  source 1..21: mol_type = other RNA
cgctactctctctttctggcc

SEQ ID NO: 18 - RNA, 21 nt, synthetic construct
  misc_feature 1..21: note = Description of Artificial Sequence: Synthetic oligonucleotide
  source 1..21: mol_type = other RNA
agcgcgagcacagctaaggcc

SEQ ID NO: 19 - RNA, 21 nt, synthetic construct
  misc_feature 1..21: note = Description of Artificial Sequence: Synthetic oligonucleotide
  source 1..21: mol_type = other RNA
ttttgtctgtgatatacacat

SEQ ID NO: 20 - RNA, 21 nt, synthetic construct
  misc_feature 1..21: note = Description of Artificial Sequence: Synthetic oligonucleotide
  source 1..21: mol_type = other RNA
cttgacagcggaagtggttgc

SEQ ID NO: 21 - RNA, 21 nt, synthetic construct
  misc_feature 1..21: note = Description of Artificial Sequence: Synthetic oligonucleotide
  source 1..21: mol_type = other RNA
tgggtcagggccagggccccc

SEQ ID NO: 22 - RNA, 21 nt, synthetic construct
  misc_feature 1..21: note = Description of Artificial Sequence: Synthetic oligonucleotide
  source 1..21: mol_type = other RNA
ccctgctctgggcttctgggt

SEQ ID NO: 23 - DNA, 1707 bp, synthetic construct; identical to SEQ ID NO: 7
  misc_feature 1..1707: note = Description of Artificial Sequence: Synthetic polynucleotide
  source 1..1707: mol_type = other DNA

SEQ ID NO: 24 - DNA, 69 bp, synthetic construct
  misc_feature 1..69: note = Description of Artificial Sequence: Synthetic oligonucleotide
  source 1..69: mol_type = other DNA
tcttaagatctttgctttcccttctctataacttcgtatagcatacattatacgaagttatattcgtct

SEQ ID NO: 25 - protein, 1053 aa, synthetic construct
  REGION 1..1053: note = Description of Artificial Sequence: Synthetic polypeptide
MKRNYILGLEIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 26 - protein, 1053 aa, synthetic construct
  REGION 1..1053: note = Description of Artificial Sequence: Synthetic polypeptide
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 27 - protein, 1368 aa, synthetic construct
  REGION 1..1368: note = Description of Artificial Sequence: Synthetic polypeptide
MDKKYSIGLEIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFESPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 28 - protein, 1368 aa, synthetic construct
  REGION 1..1368: note = Description of Artificial Sequence: Synthetic polypeptide
MDKKYSIGLEIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFESPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 29 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGTGGSGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 30 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETFKAKMLKLGPDGRKALYSKPGSKNGRWNPETHKFCKCGVRIQTSAYTCSKCRNGGSGGTGGSGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 31 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSRPGSKSGRWNPETHKFCKCGVRIQTSAYTCSKCRNGGSGGTGGSGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 32 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETFKAKMLKLGPDGRKALYSRPGSKSGRWNPETHKFCKCGVRIQTSAYTCSKCRNGGSGGTGGSGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 33 - protein, 1232 aa, synthetic construct
  REGION 1..1232: note = Description of Artificial Sequence: Synthetic polypeptide
MGKSGIYQIKNTLNNKVYVGSAKDFERRWKRHFKDLEKGCHSSIKLQRSFNKHGNVFECSILEEIPYEKDLIIERENFWIKELNSKINGYNIADASFGDTCSTHPLKEEIIKKRSETVKAKMLKLGPDGRKALYSKPGSKNGRWNPETHKFCKCGVRIRTSAYTCSKCRNGGSGGTGGSGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 34 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGTGGSGKRNYILGLEIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 35 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETFKAKMLKLGPDGRKALYSKPGSKNGRWNPETHKFCKCGVRIQTSAYTCSKCRNGGSGGTGGSGKRNYILGLEIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 36 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSRPGSKSGRWNPETHKFCKCGVRIQTSAYTCSKCRNGGSGGTGGSGKRNYILGLEIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 37 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETFKAKMLKLGPDGRKALYSRPGSKSGRWNPETHKFCKCGVRIQTSAYTCSKCRNGGSGGTGGSGKRNYILGLEIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 38 - protein, 1232 aa, synthetic construct
  REGION 1..1232: note = Description of Artificial Sequence: Synthetic polypeptide
MGKSGIYQIKNTLNNKVYVGSAKDFERRWKRHFKDLEKGCHSSIKLQRSFNKHGNVFECSILEEIPYEKDLIIERENFWIKELNSKINGYNIADASFGDTCSTHPLKEEIIKKRSETVKAKMLKLGPDGRKALYSKPGSKNGRWNPETHKFCKCGVRIRTSAYTCSKCRNGGSGGTGGSGKRNYILGLEIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 39 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGTGGSGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 40 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETFKAKMLKLGPDGRKALYSKPGSKNGRWNPETHKFCKCGVRIQTSAYTCSKCRNGGSGGTGGSGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 41 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSRPGSKSGRWNPETHKFCKCGVRIQTSAYTCSKCRNGGSGGTGGSGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 42 - protein, 1232 aa, synthetic construct
  REGION 1..1232: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETFKAKMLKLGPDGRKALYSRPGSKSGRWNPETHKFCKCGVRIQTSAYTCSKCRNGGSGGTGGSGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 43 - protein, 1232 aa, synthetic construct
  REGION 1..1232: note = Description of Artificial Sequence: Synthetic polypeptide
MGKSGIYQIKNTLNNKVYVGSAKDFERRWKRHFKDLEKGCHSSIKLQRSFNKHGNVFECSILEEIPYEKDLIIERENFWIKELNSKINGYNIADASFGDTCSTHPLKEEIIKKRSETVKAKMLKLGPDGRKALYSKPGSKNGRWNPETHKFCKCGVRIRTSAYTCSKCRNGGSGGTGGSGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 44 - protein, 1486 aa, synthetic construct
  REGION 1..1486: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGTGGSGTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 45 - protein, 1486 aa, synthetic construct
  REGION 1..1486: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETFKAKMLKLGPDGRKALYSKPGSKNGRWNPETHKFCKCGVRIQTSAYTCSKCRNGGSGGTGGSGTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 46 - protein, 1486 aa, synthetic construct
  REGION 1..1486: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSRPGSKSGRWNPETHKFCKCGVRIQTSAYTCSKCRNGGSGGTGGSGTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 47 - protein, 1486 aa, synthetic construct
  REGION 1..1486: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETFKAKMLKLGPDGRKALYSRPGSKSGRWNPETHKFCKCGVRIQTSAYTCSKCRNGGSGGTGGSGTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 48 - protein, 1486 aa, synthetic construct
  REGION 1..1486: note = Description of Artificial Sequence: Synthetic polypeptide
MGKSGIYQIKNTLNNKVYVGSAKDFERRWKRHFKDLEKGCHSSIKLQRSFNKHGNVFECSILEEIPYEKDLIIERENFWIKELNSKINGYNIADASFGDTCSTHPLKEEIIKKRSETVKAKMLKLGPDGRKALYSKPGSKNGRWNPETHKFCKCGVRIRTSAYTCSKCRNGGSGGTGGSGTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 49 - protein, 1156 aa, synthetic construct
  REGION 1..1156: note = Description of Artificial Sequence: Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGTGGSQEIKRINKIRRRLVKDSNTKKAGKTGPMKTLLVRVMTPDLRERLENLRKKPENIPQPISNTSRANLNKLLTDYTEMKKAILHVYWEEFQKDPVGLMSRVAQPAPKNIDQRKLIPVKDGNERLTSSGFACSQCCQPLYVYKLEQVNDKGKPHTNYFGRCNVSEHERLILLSPHKPEANDELVTYSLGKFGQRALDFYSIHVTRESNHPVKPLEQIGGNSCASGPVGKALSDACMGAVASFLTKYQDIILEHQKVIKKNEKRLANLKDIASANGLAFPKITLPPQPHTKEGIEAYNNVVAQIVIWVNLNLWQKLKIGRDEAKPLQRLKGFPSFPLVERQANEVDWWDMVCNVKKLINEKKEDGKVFWQNLAGYKRQEALLPYLSSEEDRKKGKKFARYQFGDLLLHLEKKHGEDWGKVYDEAWERIDKKVEGLSKHIKLEEERRSEDAQSKAALTDWLRAKASFVIEGLKEADKDEFCRCELKLQKWYGDLRGKPFAIEAENSILDISGFSKQYNCAFIWQKDGVKKLNLYLIINYFKGGKLRFKKIKPEAFEANRFYTVINKKSGEIVPMEVNFNFDDPNLIILPLAFGKRQGREFIWNDLLSLETGSLKLANGRVIEKTLYNRRTRQDEPALFVALTFERREVLDSSNIKPMNLIGIDRGENIPAVIALTDPEGCPLSRFKDSLGNPTHILRIGESYKEKQRTIQAAKEVEQRRAGGYSRKYASKAKNLADDMVRNTARDLLYYAVTQDAMLIFENLSRGFGRQGKRTFMAERQYTRMEDWLTAKLAYEGLPSKTYLSKTLAQYTSKTCSNCGFTITSADYDRVLEKLKKTATGWMTTINGKELKVEGQITYYNRYKRQNVVKDLSVELDRLSEESVNNDISSWTKGRSGEALSLLKKRFSHRPVQEKFVCLNCGFETHADEQAALNIARSWLFLRSQEYKKYQTNKTTGNTDKRAFVETWQSFYRKKLKEVWKPAV

SEQ ID NO: 50 - RNA, 20 nt, synthetic construct
  misc_feature 1..20: note = Description of Artificial Sequence: Synthetic oligonucleotide
  source 1..20: mol_type = other RNA
taatttctactcttgtagat

SEQ ID NO: 51 - protein, 1306 aa, unidentified
  REGION 1..1306: note = Description of Unknown: Cas12a sequence
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 52 - protein, 977 aa, unidentified
  REGION 1..977: note = Description of Unknown: CasX sequence
QEIKRINKIRRRLVKDSNTKKAGKTGPMKTLLVRVMTPDLRERLENLRKKPENIPQPISNTSRANLNKLLTDYTEMKKAILHVYWEEFQKDPVGLMSRVAQPAPKNIDQRKLIPVKDGNERLTSSGFACSQCCQPLYVYKLEQVNDKGKPHTNYFGRCNVSEHERLILLSPHKPEANDELVTYSLGKFGQRALDFYSIHVTRESNHPVKPLEQIGGNSCASGPVGKALSDACMGAVASFLTKYQDIILEHQKVIKKNEKRLANLKDIASANGLAFPKITLPPQPHTKEGIEAYNNVVAQIVIWVNLNLWQKLKIGRDEAKPLQRLKGFPSFPLVERQANEVDWWDMVCNVKKLINEKKEDGKVFWQNLAGYKRQEALLPYLSSEEDRKKGKKFARYQFGDLLLHLEKKHGEDWGKVYDEAWERIDKKVEGLSKHIKLEEERRSEDAQSKAALTDWLRAKASFVIEGLKEADKDEFCRCELKLQKWYGDLRGKPFAIEAENSILDISGFSKQYNCAFIWQKDGVKKLNLYLIINYFKGGKLRFKKIKPEAFEANRFYTVINKKSGEIVPMEVNFNFDDPNLIILPLAFGKRQGREFIWNDLLSLETGSLKLANGRVIEKTLYNRRTRQDEPALFVALTFERREVLDSSNIKPMNLIGIDRGENIPAVIALTDPEGCPLSRFKDSLGNPTHILRIGESYKEKQRTIQAAKEVEQRRAGGYSRKYASKAKNLADDMVRNTARDLLYYAVTQDAMLIFENLSRGFGRQGKRTFMAERQYTRMEDWLTAKLAYEGLPSKTYLSKTLAQYTSKTCSNCGFTITSADYDRVLEKLKKTATGWMTTINGKELKVEGQITYYNRYKRQNVVKDLSVELDRLSEESVNNDISSWTKGRSGEALSLLKKRFSHRPVQEKFVCLNCGFETHADEQAALNIARSWLFLRSQEYKKYQTNKTTGNTDKRAFVETWQSFYRKKLKEVWKPAV

SEQ ID NO: 53 - protein, 169 aa, unidentified
  REGION 1..169: note = Description of Unknown: I-TevI nuclease domain and linker domain sequence
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRN

SEQ ID NO: 54 - protein, 1052 aa, unidentified
  REGION 1..1052: note = Description of Unknown: CAS9 sequence
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 55 - protein, 7 aa, unidentified
PKKKRKV

SEQ ID NO: 56 - protein, 16 aa, unidentified
  REGION 1..16: note = Description of Unknown: Nucleoplasmin nuclear localization sequence
KRPAATKKAGQAKKKK

SEQ ID NO: 57 - protein, 77 aa, unidentified
  REGION 1..77: note = Description of Unknown: I-TevI linker fragment sequence
DATFGDTCSTHPLKEEIIKKRSETVKAKMLKLGPDGRKALYSKPGSKNGRWNPETHKFCKCGVRIQTSAYTCSKCRN

SEQ ID NO: 58 - protein, 93 aa, unidentified; identical to SEQ ID NO: 8
  REGION 1..93: note = Description of Unknown: ITev-I nuclease domain sequence

SEQ ID NO: 59 - protein, 83 aa, unidentified
  REGION 1..83: note = Description of Unknown: ITev-I linker domain sequence
DATFGDTCSTHPLKEEIIKKRSETVKAKMLKLGPDGRKALYSKPGSKNGRWNPETHKFCKCGVRIQTSAYTCSKCRNGGSGGS

SEQ ID NO: 60 - DNA, 1709 bp, synthetic construct
  misc_feature 1..1709: note = Description of Artificial Sequence: Synthetic polynucleotide
  source 1..1709: mol_type = other DNA
ttagcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctcggctttccccccatctcagggtcccggccacgtctcttgtccaaaacatcgtactcctctcttcgtcctagattgagctcgttatagagctggttctggccctgctggtacgcgggggcgtctgcgctcctgctgaacttcactctggagcgataggctgcgaagtcgcgtggtggggcatagggctggtaatgcttgcgggtgggcccggggcggcggggagtcatgttcatgtagtcactgtgcaggagcctgctcctcttactcctcacccagaaaataataaaggccactgttactagcaagctatagcaagccaggactcccccaaccaccaccagcacccaaaagggcttagaaggtccgggaaataggggacttggacaaaggtgtttccctttcacatggataatggttccattgctcttctcattgtctaggtaaggaggaggatacataacttcaattgcggccgctgaggagacggtgactgaggttccttgaccccagtagtccatagcatagctaccaccgtagtaataatgtttggcacagtagtaaatggctgtgtcatcagtttgcagactgttcatttttaagaaaacttggctcttggagttgtccttgatgatggtcagtctggatttgagagctgaattatagtatgtggtttcactaccccatattactcccagccactccagaccctttcgtggaggctggcgaatccagcttacaccatagtcgggtaatgagacccctgagacagtgcatgtgacggacaggctctgtgagggcgccaccaggccaggtcctgactcctgcagtttcacctcgcccttggtggatccctcgccagatccgggcttgccggatccagaggtggagcctgttatttccaacttagtcccccctccgaacgtgtacggaagcgtattaccctgttggcaaaagtaagtggcaatatcttcttgctccaggttgctaatggtgagagaataatctgttccagacccactgccactgaaccttgatgggactcctgagtgtaatcttgatgtatggtagatcaggagtttaacagttccatctggtttctgctgataccaatttaaatatttactaatgtcctgacttgccctgcaactgatggtgactctgtctcccagagaggcagacagggaggatgtagtctgtgtcatctggatgtctgggatcaggaggaatgctgggtgtggtaactcacagagcagaaggcttgtcaccaggagaagcatggtggcggatcccgcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgccccc

SEQ ID NO: 61 - DNA, 71 bp, synthetic construct
  misc_feature 1..71: note = Description of Artificial Sequence: Synthetic oligonucleotide
  source 1..71: mol_type = other DNA
agacgaatataacttcgtataatgtatgctatacgaagttatagagaagggaaagcaaagatcttaagacc

SEQ ID NO: 62 - DNA, 46 bp, Homo sapiens
  source 1..46: mol_type = other DNA
agagcagggtcccgcttccctaaggccctgctctgggcttctgggt

SEQ ID NO: 63 - DNA, 46 bp, synthetic construct
  misc_feature 1..46: note = Description of Artificial Sequence: Synthetic oligonucleotide
  source 1..46: mol_type = other DNA
accacatcatatcttggttgcaagggcctgggctgggcttctgggt

SEQ ID NO: 64 - DNA, 46 bp, synthetic construct
  misc_feature 1..46: note = Description of Artificial Sequence: Synthetic oligonucleotide
  source 1..46: mol_type = other DNA
cccaggaaccatgcttcccaggctcccttgcccttggcttctgggt

SEQ ID NO: 65 - DNA, 46 bp, synthetic construct
  misc_feature 1..46: note = Description of Artificial Sequence: Synthetic oligonucleotide
  source 1..46: mol_type = other DNA
actgggagctggtgctgtatcatgcccctgctctgagattctgggt

SEQ ID NO: 66 - DNA, 46 bp, synthetic construct
  misc_feature 1..46: note = Description of Artificial Sequence: Synthetic oligonucleotide
  source 1..46: mol_type = other DNA
tgattgagctttgcagcaaatgttttccttctcttgacttctgggt

SEQ ID NO: 67 - DNA, 46 bp, synthetic construct
  misc_feature 1..46: note = Description of Artificial Sequence: Synthetic oligonucleotide
  source 1..46: mol_type = other DNA
ataacatgtagcaattataaaaattaccttctctgaacttctgggt

(19) United States
(12) Patent Application Publication
(10) Pub. No.: US 2024/0150796 A1
(43) Pub. Date: May 9, 2024

(54) GENE EDITING WITH A MODIFIED ENDONUCLEASE

(71) Applicant: Specific Biologics Inc., Toronto (CA)

(72) Inventors: Brent E. STEAD, Toronto (CA); Thomas A. MCMURROUGH, Toronto (CA); Odisho K. ISRAEL, Toronto (CA)

(21) Appl. No.: 18/310,587

(22) Filed: May 2, 2023

Related U.S. Application Data
(63) Continuation of application No. PCT/IB2021/060236, filed on Nov. 4, 2021.
(60) Provisional application No. 63/109,396, filed on Nov. 4, 2020.

(51) Int. Cl. C12N 15/90; A61K 48/00; C12N 9/22; C12N 15/11

(52) U.S. Cl. CPC C12N 15/907; A61K 48/0075; C12N 9/22; C12N 15/11; C07K 2319/09; C07K 2319/81

(57) ABSTRACT

An engineered nuclease with unique binding and cleavage properties and methods to alter cellular gene expression using said modified endonucleases for gene editing.

### CROSS-REFERENCE

This application is a continuation of International Application No. PCT/IB2021/060236, filed on Nov. 4, 2021, which claims the benefit of U.S. Provisional App. Ser. No. 63/109,396 filed on Nov. 4, 2020, each of which is incorporated by reference herein in its entirety.

This application contains a Sequence Listing, which is incorporated by reference in its entirety. The accompanying Sequence Listing text file, name, “062709-502C01US_SL_ST26.xml” was created on Nov. 22, 2023 and is 136 KB.

### BACKGROUND

Cell therapies inject healthy human cells to regenerate damage tissues, produce missing or malfunctioning enzymes or proteins, or leverage immune cell functions as a medicinal treatment to a myriad of diseases including cancer. Cancer affects roughly ⅓ of Americans in their lifetime and is expected to rise. Cancer can occur in nearly every organ system including breast, lung, prostate, color, bladder skin, and blood. Most treatments are administered heavy doses of toxic substances (chemotherapeutics and radiation) that have off-target and wide-range side effects and often include invasive excisions of affected tissues. Immunotherapy uses a patient's own immune system to target and kill cancerous cells. Cellular immunotherapy involves programming a patient's immune cells, typically through gene editing, to target such cells to selectively kill cancerous cells. Modifying a patient's immune cells to attack tumors has the potential to be an effective, and less invasive treatment. Engineered cell therapies to regenerate damaged tissue or produce missing or malfunctioning enzymes also use a patient's own cells and often induced pluripotent stem cells which are programmed through gene editing technologies. Applications of gene-editing technologies could enable changes in a patient's immune cells or stem cells and used as part of a therapy or treatment for a variety of diseases and conditions.

Gene editing is a gene therapy approach that relies on designer nucleases to recognize and cut specific DNA sequences, and subsequently exploits innate cellular DNA repair pathways, namely nonhomologous end joining (NHEJ) and homology directed repair (HDR), to introduce targeted modifications in the genome. These nucleases can be designed to precisely introduce a double stranded break at the target locus of interest. Gene editing opens up the possibility of permanently modifying a genomic sequence of interest by enabling targeted disruption, insertion, excision, and correction in both ex vivo and in vivo settings. Existing gene editing technologies, such as CRISPR-Cas9 (and Cas9 fusions), meganucleases, zinc finger proteins, type IIS restriction endonucleases (Fokl and Fokl fusions) and TALENS are limited in the ability to introduce gene deletions of a specific length or to accurately repair a target gene in a sufficient number of cells to be meaningful as a therapeutic agent for many genetic diseases. Moreover, for highly programmable RNA-guided nucleases, such as the monomeric Cas9, studies suggest that the specificity for predictably binding, cleaving and repairing only their target sites is limited, raising concerns over potential deleterious changes to a cell's genomic DNA that may inadvertently cause a secondary disease in a patient.

A component of the type II CRISPR system that constitutes the innate immune system of bacteria, the Cas9 (CRISPR-associated) protein, has caused a paradigm shift in the field of genome editing due to its ease-of-use. Programming Cas9 to cleave a desired sequence is a simple matter of changing the sequence of the Cas9-associated guide RNA to be complementary to the target site. The ease of programming Cas9 targeting contrasts with the more intensive protein engineering that is required for other reagents (zinc finger nucleases (ZFNs), meganucleases, transcription activator-like effector nucleases (TALENs)). Cas9, along with proteins from type II CRISPR systems, have been used for a myriad of genome-editing applications in a diverse range of organisms and are now entering the realm of therapeutic applications in humans. International patent application (PCT/M2020/054229, the contents of which are incorporated by reference herein), published in the name of Specific Biologics (Toronto, Ontario, CA), describes a version of the dual-cleaving chimeric nuclease TevSaCas9 which comprises amino acids 1-92 of the I-TevI nuclease domain, a linker region comprising amino acids 93-169 of I-TevI linker region and a Staphylococcus aureus Cas9 (“saCas9”) domain. It also describes methods to manufacture the TevSaCas9. Modifying TevSaCas9 to modify immune cells into targeting cancer related could make cancer treatment cell therapies more efficient and effective. Further, modifying TevSaCas9 to modify stem cells could make treatment of damaged tissue or the replacement of missing enzymes or proteins more effective.

The immune system recognizes foreign objects and/or cells by identifying surface proteins (antigens). Receptors present on the surface of T cells target antigens and activate T cells as well as initiate other downstream immune pathways. Chimeric antigen receptor T cells (CAR-T cells) are genetically modified T cells that that produce artificial T cell receptors (TCR) used in immunotherapy and the treatment of blood and solid tumors. CAR-T cell therapy is a type of cell therapy in which T cells are harvested from patients and genetically altered to recognize antigens specific to cancerous cells. The modified cells are injected back into patients to target and destroy cancerous tissues. T cells can be collected from patient's blood (autologous) or that of a healthy donor (allogenic). Upon antigen recognition, stimulated CAR-T cells increase proliferation, cytotoxicity, and secretion of additional factors like cytokines, interleukins, and growth factors to activate other cells types. It is desirable to knockout genes in cell therapies (such as chimeric antigen receptor T-cell therapies) to limit cell exhaustion, toxicity, and insertional oncogenesis (Lui J et al., (2019), Front Immunol, 10(456):1-8).

Current technologies rely primarily on use of viral vectors for the generation of cell therapies. The use of viral vectors is highly efficient; however, questions regarding safety remain. Random insertions used to modify cell therapies can disrupt off-target endogenous gene function and lead to tumorigenesis (i.e. cancer) and cellular toxicity. A safe and efficient tool is required for the precise disruption of target genes and integration of foreign DNA to produce effective cell therapies. Applying CRISPR technology to cell therapies enables better modification of chimeric antigen receptors and endogenous genes of CAR T cells and overcomes the current limitations of T cell therapies. While these advances are expected to revolutionize the field at large, current gene editing approaches are limited by efficacy of modification, safety concerns related to the specificity of nucleases, and delivery of gene-editing tools to target cell types. The first iteration of this invention has been intentionally designed to modify the DNA of immune cells (such as T-cells) or induced pluripotent stem cells (iPSCs) to engineer them for gene knock-out (both individually and simultaneously) or the expression of an exogenous gene, such as a chimeric antigen receptor (CAR) with increased efficiency. The invention may also be used to engineer other cell types.

### SUMMARY

Described herein is a chimeric nuclease (referred to as “TevSaCas9”) comprising a modified version of an I-TevI domain, a linker peptide and a modified version of an RNA-guided nuclease Staphylococcus aureus Cas9 (“SaCas9”) domain designed to target genes to eliminate host rejection and increase the effectiveness of cell therapies. When delivered to targeted cells, the novel chimeric nuclease replaces DNA sequences in the presence of exogenous donor DNA or deletes DNA in the absence of exogenous donor DNA. Another aspect of the claimed invention is directed to methods to edit genes and producing cell therapies with edited genes described herein. Also described herein are chimeric nucleases formed from an I-TevI domain and a CasX or a Cas12 nuclease.

Described herein are: (a) Novel analogues of TevSaCas9 that target certain sites of the B2M, TRAC1, TRCB1 and/or HLA-A genes to generate out-of-frame deletions; (b) a formulation of the nuclease and exogenous donor DNA suitable for electroporation; (c) formulation of nucleases targeting one or more or B2M, TRAC1, TRBC1 and/or HLA-A simultaneously in a cell (multiplexed gene disruption); (d) a version of the invention that contains exogenous donor DNA that when delivered with the TevSaCas9 nuclease targeted to a safe harbor site in the AAVS1 gene is capable of integrating between the two sites targeted by the nuclease; and (e) novel analogues of a chimeric nuclease comprising a modified I-TevI domain, linker domain and RNA-guided nuclease domains.

In one aspect described herein is a method of making a genetically engineered cell composition comprising administering a chimeric nuclease comprising a modified I-TevI nuclease domain, a linker, a RNA-guided nuclease Staphylococcus aureus Cas9 domain, and a guide RNA to an ex vivo cell population, wherein the cell population comprises one or more of immune cells and pluripotent cells. In certain embodiments, the modified I-TevI nuclease domain comprises an amino acid sequence that is at least 85%, 90%, 95%, 97%, 98%, 99% identical to SEQ ID NO: 8. In certain embodiments, the modified I-TevI nuclease domain comprises a substitution selected from any one or more of T11V, V16I, N14G, E25D, K26R, R27A, E36S, K37N, G38N, C39V, S41H, L45F, F49Y, 160V, and E81I. In certain embodiments, the modified I-TevI nuclease domain comprises a K26R substitution. In certain embodiments, the modified I-TevI nuclease domain comprises SEQ ID NO: 8. In certain embodiments, the linker comprises an amino acid sequence that is at least 85%, 90%, 95%, 97%, 98%, 99% identical to any one of SEQ ID NOs: 9-14, or 59. In certain embodiments, the linker comprises a substitution selected from any one or more of T95S, S101Y, A119D, K120N, K135N, K135R, P126S, D127K, N140S, T147I, Q158R, A161V, or S165G. In certain embodiments, the linker comprises a substitution selected from any one or more of T95S, V117F, K135R, N140S, or Q158R. In certain embodiments, the linker is a peptide selected from any one of SEQ ID NOs: 9-14, or 59. In certain embodiments, the RNA-guided nuclease Staphylococcus aureus Cas9 domain comprises any one of SEQ ID NOs: 15, 16, 26, 27 or 28. In certain embodiments, the RNA-guided nuclease Staphylococcus aureus Cas9 domain comprises an amino acid sequence that is at least 85%, 90%, 95%, 97%, 98%, 99% identical, or is identical to any one of SEQ ID NOs: 15, 16, 26, 27 or 28. In certain embodiments, the RNA-guided nuclease Staphylococcus aureus Cas9 domain comprises a substitution selected from any one or more of T267A, L325F, V327I, D333G, A336S, I341L, E345D, D348N, K352E, S360A, T368A, N369E, N371E, S372P, E373K, K386T, N393R, H408N, N410S, 1414M, A415T, T438S, Y467F, N471K, D485E, M489F, E506K, R409K, T510E, N515K, Y518F, A539P, F550Y, N551H, S596A, T6021, A6115, I617V, T620K, G654E, N667D, R685K, K695Q, 1706V, K722T, A723T, K724N, M731T, F732V, K735Q, S739N, P741L, E742G, E746D, Q747D, I754D, T7551, H757R, K760Q, H761S, P778I, E781K, I783V, N784D, D785E, T786L, L787V, Y788H, K792E, D794T, T798R, L799I, V801I, N8035, L8041, N805K, G806N, D813G, K814E, L818I, 1819F, S822P, E824G, L841T, G847S, D848N, Y857H, V875I, I876V, N884K, A888V, L890R, D894G, D895H, P897L, V903I, G920D, F924L, N929Y, E936D, N937G, V941I, N942D, S943L, C945A, E947K, K951R, L952Q, S956N, N957E, Q958K, A959S, N974D, G975K, V983A, N984S, N985D, D986G, I991V, V993L, M995F, I996V, T999N, Y1000K, R1001E, E1002D, L10041, E1005K, N1006M, M1007N, D1009L, K1010S, R1011T, P1012S, P1013F, 11015L, 11016R, A1020G, S1021K, Q1024K, K1027S, E1039K, H1045K, I0148M, or K1050M. In certain embodiments, the RNA-guided nuclease Staphylococcus aureus Cas9 domain comprise a D10E substitution. In certain embodiments, the modified I-TevI nuclease domain comprises SEQ ID NO: 8, the linker comprises any one of SEQ ID NOs: 9-14, or 59 and the RNA-guided nuclease Staphylococcus aureus Cas9 domain comprises any one of SEQ ID NOs: 15, 16, 26, 27 or 28. In certain embodiments, the modified I-TevI nuclease domain comprises an amino acid sequence that is at least 85%, 90%, 95%, 97%, 98%, 99% identical, or is identical to any one of SEQ ID NO: 8, the linker domain comprises an amino acid sequence that is at least 85%, 90%, 95%, 97%, 98%, 99% identical, or is identical to any one SEQ ID NOs: 9-14 and the RNA-guided nuclease Staphylococcus aureus Cas9 domain comprises an amino acid sequence that is at least 85%, 90%, 95%, 97%, 98%, 99% identical to any one SEQ ID NOs: 15, 16, 26, 27 or 28. In certain embodiments, the chimeric nuclease has cleavage activity against an I-TevI recognition site in double-stranded DNA. In certain embodiments, said chimeric nuclease is delivered to the genomic DNA of target cells. In certain embodiments, the cell population comprises immune cells. In certain embodiments, the immune cells are T cells. In certain embodiments, the immune cells are natural killer (NK) cells. In certain embodiments, the cell population comprises pluripotent cells. In certain embodiments, the pluripotent cells are induced pluripotent cells. In certain embodiments, the pluripotent cells comprise hematopoietic stem cells or precursors thereof. In certain embodiments, the pluripotent cells comprise mesenchymal stem cells stem cells or precursors thereof. In certain embodiments, the pluripotent cells are induced pluripotent cells. In certain embodiments, said chimeric nuclease is delivered to the genomic DNA of target cells using a DNA expression cassette. In certain embodiments, said method does not use a viral vector. In certain embodiments, said chimeric nuclease is administered to said ex vivo cell population using electroporation. In certain embodiments, the electroporation is applied at a current of 1000 to 2500 V. In certain embodiments, the method further comprising administering a donor DNA to the cell population. In certain embodiments, the donor DNA comprises a blunt end and a two nucleotide 3′ overhang end. In certain embodiments, the donor DNA comprises a 5′ and a 3′ homology flanking an exogenous gene of interest. In certain embodiments, said donor DNA comprises an exogenous gene. In certain embodiments, the chimeric nuclease is administered in the absence of donor DNA. In certain embodiments, the chimeric nuclease modifies the genomic DNA of the target cells to delete one or more defined lengths of genomic DNA. In certain embodiments, said exogenous gene expresses a chimeric antigen receptor. In certain embodiments, said chimeric nuclease targets and cleaves a B2M gene, a TRAC1 gene, a TRCB1 gene, an HLA-A gene, or an HLA-B gene. In certain embodiments, the guide RNA of said chimeric nuclease comprises either SEQ ID NO:17 or SEQ ID NO:18, or a fragment thereof; wherein said chimeric nuclease targets and cleaves the B2M gene. In certain embodiments, the guide RNA of said chimeric nuclease comprises either SEQ ID NO:19, or a fragment thereof; wherein said chimeric nuclease targets and cleaves the TRAC1 gene. In certain embodiments, the guide RNA of said chimeric nuclease comprises SEQ ID NO:20, or a fragment thereof; wherein said chimeric nuclease targets TRCB1 gene. In certain embodiments, the guide RNA of said chimeric nuclease comprises SEQ ID NO:21, or a fragment thereof; wherein said chimeric nuclease targets and cleaves the HLA-A gene. In certain embodiments, the guide RNA of said chimeric nuclease comprises SEQ ID NO: 22, or a fragment thereof; wherein said chimeric nuclease targets the AAVS1 gene. In certain embodiments, the AAVS1 gene is targeted in the presence of exogenous donor DNA. In certain embodiments, the chimeric nuclease targets two sites on the AAVS1 gene. In certain embodiments, the exogenous donor DNA is integrated between the two targeted sites on the AAVS1 gene. In certain embodiments, the guide RNA comprises one or more of: a non-natural internucleoside linkage, a nucleic acid mimetic, a modified sugar moiety, and a modified nucleobase. In certain embodiments, the non-natural internucleoside linkage comprises one or more of: a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a 3′-amino phosphoramidate, an aminoalkylphosphoramidate, a phosphorodiamidate, a thionophosphoramidate, a thionoalkylphosphonate, a thionoalkylphosphotriester, a selenophosphate, and a boranophosphate. In certain embodiments, the nucleic acid mimetic comprises one or more of a peptide nucleic acid (PNA), morpholino nucleic acid, cyclohexenyl nucleic acid (CeNAs), or a locked nucleic acid (LNA). In certain embodiments, the modified sugar moiety comprises one or more of 2′-O-(2-methoxyethyl), 2′-dimethylaminooxyethoxy, 2′-dimethylaminoethoxyethoxy, 2′-O-methyl, and 2′-fluoro. In certain embodiments, the modified nucleobase comprises one or more of: a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-halouracil; a 5-halocytosine; a 5-propynyl uracil; a 5-propynyl cytosine; a 6-azo uracil; a 6-azo cytosine; a 6-azo thymine; a pseudouracil; a 4-thiouracil; an 8-halo; an 8-amino; an 8-thiol; an 8-thioalkyl; an 8-hydroxyl; a 5-halo; a 5-bromo; a 5-trifluoromethyl; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deaza-adenine; a 7-deazaguanosine; a 2-aminopyridine; a 2-pyridone; a 5-substituted pyrimidine; a 6-azapyrimidine; an N-2, N-6 or 0-6 substituted purine; a 2-aminopropyladenine; a 5-propynyluracil; and a 5-propynylcytosine.

In one aspect, described herein, is a chimeric nuclease comprising a modified I-TevI nuclease domain and/or I-TevI linker domain, a CasX polypeptide, and a guide RNA, wherein said chimeric nuclease optionally comprises a nuclear localization signal. In certain embodiments, the modified I-TevI nuclease domain and/or the I-TevI linker domain comprises a modification at one or more amino acid residues corresponding to K26, T95, V117, K135, N140, and Q158 of the unmodified I-TevI nuclease set forth in SEQ ID NO: 53. In certain embodiments, the modified I-TevI nuclease domain and/or the I-TevI linker domain comprises an amino acid modification at an amino acid corresponding to V117 of an unmodified I-TevI nuclease set forth in SEQ ID NO: 53. In certain embodiments, the modified I-TevI nuclease domain and/or the I-TevI linker domain comprises an amino acid modification corresponding to V117F of an unmodified I-TevI nuclease set forth in SEQ ID NO: 53. In certain embodiments, the modified I-TevI nuclease domain and/or the I-TevI linker domain comprises an amino acid modification at an amino acid corresponding to V117, K135, and N140 of an unmodified I-TevI nuclease set forth in SEQ ID NO: 53. In certain embodiments, the modified I-TevI nuclease domain and/or the I-TevI linker domain linker comprises an amino acid modification corresponding to V117F, K135R, and N140S of an unmodified I-TevI nuclease set forth in SEQ ID NO: 53. In certain embodiments, the modified I-TevI nuclease domain and/or the linker comprises an amino acid modification at an amino acid corresponding to K135 and N140 of an unmodified I-TevI nuclease set forth in SEQ ID NO: 53. In certain embodiments, the modified I-TevI nuclease domain and/or the I-TevI linker domain comprises an amino acid modification corresponding to K135R and a N140S of an unmodified I-TevI nuclease set forth in SEQ ID NO: 53. In certain embodiments, the modified I-TevI nuclease domain and/or the I-TevI linker domain comprises an amino acid modification at an amino acid corresponding to K26, T95, and a Q158 of an unmodified I-TevI nuclease set forth in SEQ ID NO: 53. In certain embodiments, the modified I-TevI nuclease domain and/or the linker comprises an amino acid modification corresponding to K26R, T95S, and Q158R of an unmodified I-TevI nuclease set forth in SEQ ID NO: 53. In certain embodiments, the modified I-TevI nuclease domain and/or the I-TevI linker domain comprises an amino acid sequence at least about 85%, 90%, 95%, 97%, 98%, 99% identical, or is identical to SEQ ID NO: 53. In certain embodiments, the CasX polypeptide comprises an amino acid sequence at least about 85%, 90%, 95%, 97%, 98%, 99% identical, or is identical to SEQ ID NO: 52. In certain embodiments, the Cas12 polypeptide comprises a substitution selected from any one or more of R11K, R12K, V145, K15A, S17N. N18A, A22V, G23S, T25S, P38D, K41K, E42K, N46K, L47R, N53V, I54M, P57V, T61N, S62A, R63A, A64N, E75K, H82Q, Q89K, P1045, N106K, 1113K, N199K, S124T, S125A, C133G, Y137F, N145S, D146E, H151Y, S161A, R165K, N177S, L180A, R202K, N205T, G215A, C219Y, V236I, T241S, L248I, I254V, S269G, 1290V, E291D, V297I, Q299R, 1314L, E318D, Q323L, L333V, E359D, D360M, K362R, Q366S, N367G, L368V, A369T, G370A, Y371E, H404Y, H409Y, G410A, E411G, Y417F, V428I, E429A, S432T, K433S, L437R, S443A, A451V, I464L, A470M, 1502V, L503V, I531L, G537K, L540I, N553S, I559L, S563G, V571L, N579Q, H589T, 5607L, L6081, L620I, R623K, R624K, L644V, S646P, M652V, I657V, R679E, L684S, N686G, H689D, S696G, T702A, T737S, L742F, Y744H, Q748H, M751V, I753V, A771T, R777K, P792T, S818T, R823G, V824M, E826V, K827R, A832S, T833D, M836A, I839L, G841N, V846A, N860T, V862E, D864E, V867A, V877G, S883K, S889R, G890D, S894F, K908Q, N913D, F916H, T918V, R936N, Q938N, Y940F, K942S, S963A, R966K, K967R, or K968R. In certain embodiments, the CasX polypeptide is from Planctomycetes bacterium. In certain embodiments, the CasX polypeptide is from Deltaproteobacteria. In certain embodiments, the chimeric nuclease further comprises a guide RNA. In certain embodiments, the guide RNA targets a genomic target in a mammalian cell. In certain embodiments, the guide RNA targets an oncogenic mutation in a mammalian cell. In certain embodiments, the mammalian cell is a human cell. In certain embodiments, the guide RNA targets a bacterial or a viral sequence. In certain embodiments, the guide RNA comprises one or more of: a non-natural internucleoside linkage, a nucleic acid mimetic, a modified sugar moiety, and a modified nucleobase. In certain embodiments, the non-natural internucleoside linkage comprises one or more of: a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a 3′-amino phosphoramidate, an aminoalkylphosphoramidate, a phosphorodiamidate, a thionophosphoramidate, a thionoalkylphosphonate, a thionoalkylphosphotriester, a selenophosphate, and a boranophosphate. In certain embodiments, the nucleic acid mimetic comprises one or more of a peptide nucleic acid (PNA), morpholino nucleic acid, cyclohexenyl nucleic acid (CeNAs), or a locked nucleic acid (LNA). In certain embodiments, the modified sugar moiety comprises one or more of 2′-O-(2-methoxyethyl), 2′-dimethylaminooxyethoxy, 2′-dimethylaminoethoxyethoxy, 2′-O-methyl, and 2′-fluoro. In certain embodiments, the modified nucleobase comprises one or more of: a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-halouracil; a 5-halocytosine; a 5-propynyl uracil; a 5-propynyl cytosine; a 6-azo uracil; a 6-azo cytosine; a 6-azo thymine; a pseudouracil; a 4-thiouracil; an 8-halo; an 8-amino; an 8-thiol; an 8-thioalkyl; an 8-hydroxyl; a 5-halo; a 5-bromo; a 5-trifluoromethyl; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deaza-adenine; a 7-deazaguanosine; a 2-aminopyridine; a 2-pyridone; a 5-substituted pyrimidine; a 6-azapyrimidine; an N-2, N-6 or 0-6 substituted purine; a 2-aminopropyladenine; a 5-propynyluracil; and a 5-propynylcytosine. Also described herein is a nucleic acid or a plurality of nucleic acids encoding the chimeric nuclease. In certain embodiments, the chimeric nuclease and or the guide RNA is operably coupled to one or more of a eukaryotic promoter, enhancer, or polyadenylation site. In certain embodiments, the nucleic acid is an expression vector selected from a plasmid, a lentivirus vector, and adeno associated virus vector, and an adenovirus vector. In certain embodiments, the chimeric nuclease is formulated for administration to an individual. Also described herein is a composition comprising the chimeric nuclease and a pharmaceutically acceptable excipient, diluent or carrier. Also described herein is a composition comprising the chimeric nuclease encapsulated in a lipid nanoparticle. In certain embodiments, the lipid nanoparticle comprises cationic and/or neutral lipids.

In one aspect, described herein, is a chimeric nuclease comprising a modified I-TevI nuclease domain and/or I-TevI linker domain, a Cas12 polypeptide, and a guide RNA, wherein said chimeric nuclease comprises a nuclear localization signal. In certain embodiments, the modified I-TevI nuclease domain and/or the I-TevI linker domain comprises a modification at one or more amino acid residues corresponding to K26, T95, V117, K135, N140, and Q158 of the unmodified I-TevI nuclease set forth in SEQ ID NO: 53. In certain embodiments, the modified I-TevI nuclease domain and/or the I-TevI linker domain comprises an amino acid modification at an amino acid corresponding to V117 of an unmodified I-TEVI nuclease set forth in SEQ ID NO: 53. In certain embodiments, the modified I-TevI nuclease domain and/or the I-TevI linker domain comprises an amino acid modification corresponding to V117F of an unmodified I-TEVI nuclease set forth in SEQ ID NO: 53. In certain embodiments, the modified I-TevI nuclease domain and/or the I-TevI linker domain comprises an amino acid modification at an amino acid corresponding to V117, K135, and N140 of an unmodified I-TevI nuclease set forth in SEQ ID NO: 53. In certain embodiments, the modified I-TevI nuclease domain and/or the I-TevI linker domain linker comprises an amino acid modification corresponding to V117F, K135R, and N140S of an unmodified I-TevI nuclease set forth in SEQ ID NO: 53. In certain embodiments, the modified I-TevI nuclease domain and/or the linker comprises an amino acid modification at an amino acid corresponding to K135 and N140 of an unmodified I-TevI nuclease set forth in SEQ ID NO: 53. In certain embodiments, the modified I-TevI nuclease domain and/or the I-TevI linker domain comprises an amino acid modification corresponding to K135R and a N140S of an unmodified I-TevI nuclease set forth in SEQ ID NO: 53. In certain embodiments, the modified I-TevI nuclease domain and/or the I-TevI linker domain comprises an amino acid modification at an amino acid corresponding to K26, T95, and a Q158 of an unmodified I-TevI nuclease set forth in SEQ ID NO: 53. In certain embodiments, the modified I-TevI nuclease domain and/or the linker comprises an amino acid modification corresponding to K26R, T95S, and Q158R of an unmodified I-TevI nuclease set forth in SEQ ID NO: 53. In certain embodiments, the modified I-TevI nuclease domain and/or the I-TevI linker domain comprises an amino acid sequence at least about 85%, 90%, 95%, 97%, 98%, 99% identical, or is identical to SEQ ID NO: 53. In certain embodiments, the Cas12 polypeptide is from Acidaminococcus sp. BV3L6. In certain embodiments, the Cas12 polypeptide comprises an amino acid sequence at least about 85%, 90%, 95%, 97%, 98%, 99% identical, or is identical to SEQ ID NO: 51. In certain embodiments, the Cas12 polypeptide comprises a substitution selected from any one or more of T1S, Q2N, E45, GSE, N8H, L9K, K28E, H29N, 130L, Q31T, E32A, Q33Y, F35M, I36V, E37N, E38D, A41L, N435, D44E, H45N, E48K, I52V, R55K, T59Y, Y60F, A61I, D62E, Q63E, C64T, Q66K, L67H, Q69A, L70I, N74P, S76Y, A77K, D80T, S81A, Y82F, E85D, E88L, T90N, R91N, N92T, A93N, I95R, E97I, A99D, T100N, Y101C, N103K, A1045, H106A, D107G, I110E, R112K, T113V, D114P, R159K, S169V, S185A, A1875, I192L, D195E, K2011, T212K, R218N, N223T, I228T, 5233G, I236L, E237D, V239I, F242V, Q249C, Y257F, V279T, I284V, F305Y, N3135, 5324N, I329L, S331A, T337E, L338K, L345I, E349Q, 5357L, I358A, N386D, I393V, L396A, 1400L, 5403N, V408I, Q409E, G427D, K428D, Q436A, L442I, S468V, Q469L, S472A, L473V, L479T, E487D, S488D, A497V, L510I, A516V, K522Q, Q535S, M536N, S541D, V545E, K549Q, N550Q, G552C, V557E, N559E, S586N, Y596Q, A601S, 1604L, A613D, S628N, E637T, A657D, K660R, G663N, Q665K, C673H, L683V, L697V, A711G, L717F, Q723E, A733L, E735D, Y740F, K751E, K756A, G766A, I778V, R793P, L844F, I858V, 5865T, I874L, H898N, 1903V, I916A, L931F, K941N, N945Q, V951I, S958T, V959A, D965E, I938V, H984Q, A10095, C1024Y, G10375, T1049E, G1055R, T1056N, Y1068F, L1075A, V1083R, K1085G, L1097I, H1104K, D1106N, D1111N, L1122K, A1134D, V1138I, D1147A, V1160E, P1161F, R1171Q, R1173E, Y1176L, N1205T, D1207N, S1220L, V1221T, A1230E, N1237S, L12431, M1259K, Q1274L, G1291A, Q1295N, A1299N, or L1304K. In certain embodiments, the chimeric nuclease has cleavage activity against an I-TevI recognition site in double-stranded DNA. In certain embodiments, the chimeric nuclease further comprises a guide RNA. In certain embodiments, the guide RNA targets a genomic target in a mammalian cell. In certain embodiments, the guide RNA targets an oncogenic mutation in a mammalian cell. In certain embodiments, the mammalian cell is a human cell. In certain embodiments, the guide RNA targets a bacterial or a viral sequence. In certain embodiments, the guide RNA comprises one or more of: a non-natural internucleoside linkage, a nucleic acid mimetic, a modified sugar moiety, and a modified nucleobase. In certain embodiments, the non-natural internucleoside linkage comprises one or more of: a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a 3′-amino phosphoramidate, an aminoalkylphosphoramidate, a phosphorodiamidate, a thionophosphoramidate, a thionoalkylphosphonate, a thionoalkylphosphotriester, a selenophosphate, and a boranophosphate. In certain embodiments, the nucleic acid mimetic comprises one or more of a peptide nucleic acid (PNA), morpholino nucleic acid, cyclohexenyl nucleic acid (CeNAs), or a locked nucleic acid (LNA). In certain embodiments, the modified sugar moiety comprises one or more of 2′-O-(2-methoxyethyl), 2′-dimethylaminooxyethoxy, 2′-dimethylaminoethoxyethoxy, 2′-O-methyl, and 2′-fluoro. In certain embodiments, the modified nucleobase comprises one or more of: a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-halouracil; a 5-halocytosine; a 5-propynyl uracil; a 5-propynyl cytosine; a 6-azo uracil; a 6-azo cytosine; a 6-azo thymine; a pseudouracil; a 4-thiouracil; an 8-halo; an 8-amino; an 8-thiol; an 8-thioalkyl; an 8-hydroxyl; a 5-halo; a 5-bromo; a 5-trifluoromethyl; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deaza-adenine; a 7-deazaguanosine; a 2-aminopyridine; a 2-pyridone; a 5-substituted pyrimidine; a 6-azapyrimidine; an N-2, N-6 or O-6 substituted purine; a 2-aminopropyladenine; a 5-propynyluracil; and a 5-propynylcytosine. Also described herein is a nucleic acid or a plurality of nucleic acids encoding the chimeric nuclease. In certain embodiments, the chimeric nuclease and or the guide RNA is operably coupled to one or more of a eukaryotic promoter, enhancer, or polyadenylation site. In certain embodiments, the nucleic acid is an expression vector selected from a plasmid, a lentivirus vector, and adeno associated virus vector, and an adenovirus vector. In certain embodiments, the chimeric nuclease is formulated for administration to an individual. Also described herein is a composition comprising the chimeric nuclease and a pharmaceutically acceptable excipient, diluent or carrier. Also described herein is a composition comprising the chimeric nuclease encapsulated in a lipid nanoparticle. In certain embodiments, the lipid nanoparticle comprises cationic and/or neutral lipids.

In one aspect, described herein, is a chimeric nuclease comprising a modified I-TevI nuclease domain and/or I-TevI linker domain, a modified Cas9 polypeptide, and a guide RNA, wherein the modified I-TevI nuclease domain and/or I-TevI linker domain comprises an amino acid modification corresponding to K26R, a T95S, and a Q158R of the unmodified I-TevI nuclease set forth in SEQ ID NO: 53, and the modified Cas9 polypeptide comprises an amino acid modification corresponding to D10E or of the unmodified Cas9 set forth in SEQ ID NO: 54. In certain embodiments, the modified I-TevI nuclease domain and/or I-TevI linker domain comprises an amino acid sequence at least about 80%, 90%, 95%, 97%, 98%, 99% identical, or is identical to SEQ ID NO: 53. In certain embodiments, said chimeric nuclease comprises a nuclear localization signal. In certain embodiments, the Cas9 polypeptide is from Staphylococcus aureus. In certain embodiments, the nuclear localization signal comprises an SV40 nuclear localization signal comprising the amino acid sequence SEQ ID NO: 55 (PKKKRKV). In certain embodiments, the nuclear localization signal comprises a Nucleoplasmin nuclear localization signal comprising the amino acid sequence SEQ ID NO: 56 (KRPAATKKAGQAKKKK). In certain embodiments, the modified I-TevI linker domain comprises an amino acid sequence at least about 85%, 90%, 95%, 97%, 98%, 99% identical to SEQ ID NO: 57. In certain embodiments, the modified I-TevI nuclease domain comprises an amino acid sequence at least about 85%, 90%, 95%, 97%, 98%, 99% identical to SEQ ID NO: 58. In certain embodiments, the chimeric nuclease has cleavage activity against an I-TevI recognition site in double-stranded DNA. In certain embodiments, the chimeric nuclease further comprises a guide RNA. In certain embodiments, the guide RNA targets a genomic target in a mammalian cell. In certain embodiments, the guide RNA targets an oncogenic mutation in a mammalian cell. In certain embodiments, the mammalian cell is a human cell. In certain embodiments, the guide RNA targets a bacterial or a viral sequence. In certain embodiments, the guide RNA comprises one or more of: a non-natural internucleoside linkage, a nucleic acid mimetic, a modified sugar moiety, and a modified nucleobase. In certain embodiments, the non-natural internucleoside linkage comprises one or more of: a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a 3′-amino phosphoramidate, an aminoalkylphosphoramidate, a phosphorodiamidate, a thionophosphoramidate, a thionoalkylphosphonate, a thionoalkylphosphotriester, a selenophosphate, and a boranophosphate. In certain embodiments, the nucleic acid mimetic comprises one or more of a peptide nucleic acid (PNA), morpholino nucleic acid, cyclohexenyl nucleic acid (CeNAs), or a locked nucleic acid (LNA). In certain embodiments, the modified sugar moiety comprises one or more of 2′-O-(2-methoxyethyl), 2′-dimethylaminooxyethoxy, 2′-dimethylaminoethoxyethoxy, 2′-O-methyl, and 2′-fluoro. In certain embodiments, the modified nucleobase comprises one or more of: a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-halouracil; a 5-halocytosine; a 5-propynyl uracil; a 5-propynyl cytosine; a 6-azo uracil; a 6-azo cytosine; a 6-azo thymine; a pseudouracil; a 4-thiouracil; an 8-halo; an 8-amino; an 8-thiol; an 8-thioalkyl; an 8-hydroxyl; a 5-halo; a 5-bromo; a 5-trifluoromethyl; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deaza-adenine; a 7-deazaguanosine; a 2-aminopyridine; a 2-pyridone; a 5-substituted pyrimidine; a 6-azapyrimidine; an N-2, N-6 or 0-6 substituted purine; a 2-aminopropyladenine; a 5-propynyluracil; and a 5-propynylcytosine. Also described herein is a nucleic acid or a plurality of nucleic acids encoding the chimeric nuclease. In certain embodiments, the chimeric nuclease and or the guide RNA is operably coupled to one or more of a eukaryotic promoter, enhancer, or polyadenylation site. In certain embodiments, the nucleic acid is an expression vector selected from a plasmid, a lentivirus vector, and adeno associated virus vector, and an adenovirus vector. In certain embodiments, the chimeric nuclease is formulated for administration to an individual. Also described herein is a composition comprising the chimeric nuclease and a pharmaceutically acceptable excipient, diluent or carrier. Also described herein is a composition comprising the chimeric nuclease encapsulated in a lipid nanoparticle. In certain embodiments, the lipid nanoparticle comprises cationic and/or neutral lipids.

### BRIEF DESCRIPTION OF THE DRAWINGS

The following detailed description of the invention reference is made to the accompanying drawings which form a part hereof, and in which are shown, by way of illustration, specific embodiments in which the invention may be practiced. These embodiments are described in sufficient detail to enable those skilled in the art to practice the invention. Other embodiments may be utilized, and structural and logical changes may be made, without departing from the scope of the present invention. Reference will now be made, by way of example only, to the accompanying drawings in which:

FIGS. 1A to 1G depict a diagram of the mechanism by which TevSaCas9 is complexed with multiple guide RNAs and electroporated into cells to disrupt genes encoding the endogenous T-cell receptor and insert a sequence coding for an exogenous chimeric antigen receptor (CAR).

FIGS. 2A and 2B illustrate that TevSaCas9 targeted to the B2M gene cleaves the B2M gene. FIG. 2C evidences editing at the B2M gene by plasmid DNA encoded TevSaCas9 as detected by PCR amplification and T7 Endonuclease I cleavage assay of genomic DNA extracted from harvested cells. FIG. 2D evidences editing at the B2M gene using purified TevSaCas9 protein of SEQ ID 29 or TevSaCas9 protein of SEQ ID 32 containing the V117F/K135R/N140S mutations complexed with guide RNA targeting B2M of SEQ ID 17 and transfected into mammalian cells.

FIGS. 3A and 3B illustrate that TevSaCas9 targeted to the AAVS1 gene cleaves the AAVS1 gene. FIG. 3C evidences the activity of Tev[VKN]-SaCas9[D10E] of SEQ ID 37 and Tev[KTQ]-SaCas9[WT] of SEQ ID 33 at the AAVS1 gene in mammalian cells.

FIGS. 4A and 4B show a diagram representing the target site in the B2M gene after TevSaCas9 cleavage (black) and a donor repair template (grey) insertion into the cut site and that repair occurs.

FIGS. 5A and 5B is a diagram depicting the AAVS1 safe harbour site within the genome (black) after cleavage by TevSaCas9, and shows expression of GFP reporter inserted into the cut site.

FIG. 6 illustrates that TevSaCas9 targeted to the TRBC1 gene using guide in SEQ ID 20 cleaves the TRBC1 target site in the genome of mammalian cells.

FIG. 7A illustrate insertion of the double strand DNA oligonucleotide containing a single LoxP site into the AAVS1 target site. FIG. 7B evidences the insertion of the double strand DNA oligonucleotide containing a single LoxP site into the AAVS1 target site.

FIGS. 8A and 8B illustrates that electroporated ribonucleoprotein TevSaCas9 edits the B2M target site in mammalian cells. FIG. 8C evidences that induced donor DNA can be incorporated into the B2M target site in pluripotent stem cells (iPSCs).

FIGS. 9A and 9B illustrates that a fusion of I-TevI to Cas12a is activated by guide RNA to cleave double-strand DNA substrate.

FIG. 10 illustrates cleavage of the B2M gene by TevSaCas9 of HEK293 cells with different nuclear localization signals (NLSs).

### DETAILED DESCRIPTION

In the following description, certain details are set forth such as specific quantities, sizes, etc., so as to provide a thorough understanding of the present embodiments disclosed herein. However, it will be obvious to those skilled in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

### Methods of Ex Vivo Gene Editing

This disclosure describes chimeric nucleases and method of ex vivo gene editing that exhibit the following advantages over existing gene editing technologies and methods. For example, when and if the nuclease cleaves at two sites, it cleaves out precise lengths of DNA (˜30-38 bases depending on the sites targeted by I-TevI and SaCas9). The methods described herein may generate precise deletions of 30, 31, 32, 33, 34, 35, 36, 37, 38, 39, or 40 nucleotides from a genome. Thus, a target site can be selected in a gene to generate a precise-length deletion that will knock the gene out-of-frame in a cell. In one embodiment of the described nucleases, a nuclease targets and cleaves the B2M gene (SEQ ID NO: 1) (also known as Beta Chain of MHC Class I Molecules, Beta-2-Microglobin, IMD43) to disrupt the reading frame of B2M with multiple out-of-frame deletions. In another embodiment of the described nucleases, a nuclease targets and cleaves the B2M gene (SEQ ID NO 2) to disrupt the reading frame with a single out-of-frame deletion. In yet another embodiment of the described nucleases, a nuclease targets and cleaves the TRAC gene (SEQ ID NO 3) (also known as TRA, IMD7, TCRA, TRCA, T-cell receptor alpha locus, TCRD and T cell receptor locus) so as to disrupt the reading frame with a single-out-of-frame deletion. Another embodiment of the described nucleases, a nuclease targets and cleaves the TRCB gene (SEQ ID NO 4) (also known as T cell receptor beta locus, TCRBC1, BV05S1J2.2) so as to disrupt the reading frame with a single out-of-frame deletion. Another version of the described nucleases target and cleave the HLA-A gene (SEQ ID NO 5) (also known as Major Histocompatibility Complex, Class I, A, HLA Class I Histocompatibility Antigen, A Alpha Chain, HLAA, HLA Class I Histocompatibility Antigen, A-1 Alpha Chain, MEW Class I Antigen HLA-A Heavy Chain, Leukocyte Antigen Class I-A and Human Leukocyte Antigen A) so as to disrupt the reading frame with a single, out-of-frame deletion. A combination of two or more of the nucleases described above can simultaneously disrupt the reading frame of the respective genes in a cell. In the presence of exogenous donor DNA, the described nucleases are designed to replace target DNA sequences in a higher percentage of cells than existing technologies or practices. A further embodiment of the described nucleases target and cleave the AAVS1 locus (SEQ ID NO 6) (also known as Adeno-Associated Virus Integration Site 1 and AAV). Another version of the embodiment described herein[0026], i.e. a nuclease that targets and cleaves the AAVS1 locus (SEQ ID NO 7), is directed to a double-stranded section of donor DNA with ends that complement the nuclease cut site to target DNA insertion.

This disclosure describes methods of using the described nucleases to genetically engineer a cell population in order to insert modify, delete, or replace a nucleotides sequence at a genomic location. The steps comprise forming a chimeric nuclease guide RNA complex and administering the chimeric nuclease guide RNA complex to the cells. This administration can occur using one or more methods of electroporation or lipid mediated transfection (e.g., cationic lipids). Alternatively, a nucleic acid or plurality of nucleic acids encoding the guide RNA and/or the chimeric nuclease can be transferred into the cell using a method selected from electroporation, viral transduction, and or lipid mediated transfection can be utilized. In embodiments, where a genome sequence is to be added to alter the genome a donor DNA can also be administered to effect the insertion or alteration. The donor DNA can be suitably provided as a linearized DNA, plasmid DNA or a viral vector.

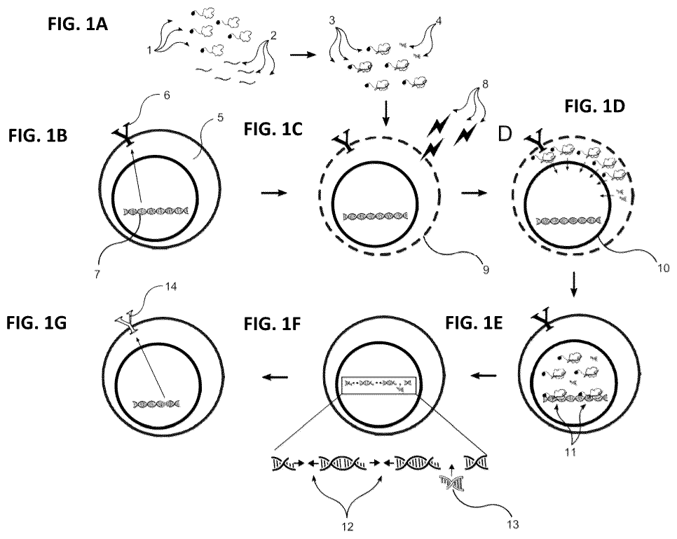

In one aspect the ex vivo gene editing methods described herein are used to produce CAR T or CAR NK cells. As illustrated in FIG. 1A, purified TevSaCas9 protein 1 is mixed complexed with multiple guide RNAs 2 to form ribonucleoprotein complex 3. Donor DNA 4 encoding an exogenous donor CAR containing complimentary ends to one or more of the sites targeted by TevSaCas9 is also mixed with the ribonucleoprotein complex 3. As shown in FIG. 1B, a population of T cells 5 which encode for an endogenous T-cell receptor 6 expressed from the genomic DNA 7 is exposed to the mixture of ribonucleoprotein complex 3 and donor DNA 4. An electrical pulse 8 is applied to the mixture to permeabilize the cell membrane 9 FIG. 1C. As depicted in FIG. 1D, the ribonucleoprotein complex 3 and donor DNA 4 enter the T cell through the permeabilize cell membrane. The ribonucleoprotein complex 3 is targeted to the nucleus 10 through one or more nuclear localization sequences (“NLS”). As shown in FIG. 1E the ribonucleoprotein complex 3 will bind to its target site on the genomic DNA 7. As depicted in FIG. 1F, the ribonucleoprotein complex 3 will cleave the genomic DNA 7 to create defined length deletions 12, or if compatible donor DNA 13 is present, will insert the donor DNA 13 into the cleaved site. The regions of the genomic DNA 7 deleted disrupt genes encoding the endogenous T-cell receptor 6 and the donor DNA 13 encodes for an exogenous CAR 14 to target the T-cell toward a new antigen.

The claimed polypeptides are designed to modify the DNA of immune cells (such as T-cells), hematopoietic stems cells, mesenchymal stem cells, or induced pluripotent stem cells (iPSCs) engineering said cells for immune tolerance and/or to express exogenous genes, such as a chimeric antigen receptor (CAR), although not limited to just those cell types. Cell types that can be ex vivo modified by the methods and nuclease described herein include immune cells such as T cell or NK-cells; or pluripotent cells such as mesenchymal stem cells, hematopoietic stems cell, or cell otherwise induced to pluripotency using techniques known in the art.

In addition, this disclosure is directed to: a method of targeted gene disruption of all or a portion of a DNA sequence in the genome of human cells to knock genes out-of-frame, comprising the steps: (a) exposing cells to the nuclease ex vivo; (b) applying an electric current of between 1000-2500V to the cell population to permeabilize the membrane to allow for the passage of the claimed nuclease into the cells. Other ranges of electric currents between 1000-1500V, 1501-1700V, 1701-1900V, 1901-2100V or 2101-2500V may also be applied. The nuclease may also be delivered to the cell using lipofection or polymer-based transfection or the use of a viral vector such as adeno-associated virus or lentivirus. The nuclease may further be delivered as a ribonucleoprotein complex, a DNA encoding the nuclease or as messenger RNA encoding the nuclease. In eukaryotic cells, the described nucleases (including TevSaCas9) can target the nuclei of the cells through one or more nuclear-localization sequences (“NLS”). For the application of generating the immune tolerant cells, applying a mixture of nucleases to target one or more of B2M, TRAC1, TRBC1, HLA-A or AAVS1 (SEQ ID NOs.: 1-6) in the population of cells. Specific guide RNAs to target the nuclease to a precise genomic location can be included with the nuclease, encoded by a nucleic acid, or a messenger RNA. For applications that target the replacement, repair, or insertion of a DNA into a genomic location, a donor DNA may also be included either as an isolated and purified nucleic acid, by linear double stranded DNA, by a plasmid or viral vector. A donor DNA may be provided along with the nuclease and guide RNA or separately in separate formulation or delivered by a different method compared to the delivery of the nuclease and guide RNA.

Also described herein is a method of targeted insertion of a defined length of a DNA sequence into human cells, comprising the steps of: (a) exposing cells to the novel nuclease ex vivo; (b) applying an electric current of between 1000-2500V to the cell population to permeabilize the membrane to allow for the passage of the claimed nuclease into the cells.

In eukaryotic cells, the described nucleases (including TevSaCas9) can target the nuclei of the cells through one or more nuclear-localization sequences (“NLS”). For the application of generating the immune tolerant cells, applying a mixture of nucleases to target one or more of B2M, TRAC1, TRBC1, HLA-A or AAVS1 (SEQ ID NOs.: 1-6) in the population of cells.

In the presence of exogenous donor DNA, the cell can insert the exogenous DNA sequence (in whole or in part) between the two cleaved sites in the target genomic DNA using directed-ligation through non-homologous end joining.

For the application of engineered CAR-T cells, the AAVS1 safe harbor site is targeted (SEQ ID NO: 6) by the exogenous donor DNA that contains a DNA sequence that codes for a chimeric antigen receptor (CAR) (SEQ ID NO: 7).

The instant application is directed to a novel modified TevSaCas9 nuclease comprising a chimeric nuclease containing different combinations of an I-TevI domain, a linker domain, a SaCas9 domain and a guide RNA. In particular, Chimeric nucleases may comprise: (a) an I-TevI domain comprising an amino acid sequence with at least about 80%, 85%, 90%, 95%, 97%, 98%, 99% identity or that is identical to the amino acid sequence in SEQ ID NO: 8; (b) a linker domain comprising an amino acid sequence with at least about 80%, 85%, 90%, 95%, 97%, 98%, 99% identity or that is identical to the amino acid sequence in any one of SEQ ID NO: 9-14; and/or an saCas9 comprising an amino acid sequence with at least about 80%, 85%, 90%, 95%, 97%, 98%, 99% identity or that is identical to the amino acid sequence in any one of SEQ ID NOs: 15-16.

Chimeric nucleases which target the B2M gene comprise (a) a chimeric nuclease as described above; and (b) a guide RNA comprising an RNA sequence with at least about 90%, 95%, 97%, 98%, 99% identity or is identical to the sequence of SEQ ID NO: 17 or SEQ ID NO: 18.

Chimeric nucleases which target the TRAC gene comprise: (a) a chimeric nuclease as described above; and (b) a guide RNA comprising an RNA sequence with at least about 90%, 95%, 97%, 98%, 99% identity or is identical to the sequence of SEQ ID NO: 19.

Chimeric nucleases which target the TRBC gene comprise: (a) a chimeric nuclease as described above; and (b) a guide RNA comprising an RNA sequence with at least about 90%, 95%, 97%, 98%, 99% identity or is identical to the sequence of SEQ ID NO: 20.

Chimeric nucleases which target the HLA-A gene comprise: (a) a chimeric nuclease as described above; and (b) a guide RNA comprising an RNA the sequence with at least about 90%, 95%, 97%, 98%, 99% identity or is identical to the sequence of SEQ ID NO: 21.

Chimeric nucleases which target the AAVS1 gene comprise: (a) a chimeric nuclease as described above; and (b) a guide RNA comprising an RNA sequence with at least about 90%, 95%, 97%, 98%, 99% identity or is identical to the sequence of SEQ ID NO: 22.

An embodiment of the claimed invention is a composition used to edit multiple genes simultaneously to generate immune tolerant cells. In particular, the composition is directed to a mixture of the chimeric nucleases discussed above in the preceding paragraph in combination with a mixture of guide RNAs according to sequences SEQ ID NOs: 17 and 19-21 in an equimolar ratio to the chimeric nuclease. In another embodiment, the composition is directed to a mixture of the chimeric nucleases discussed above in the preceding paragraph in combination with a mixture of guide RNAs according to sequences SEQ ID NOs: 18-21 in an equimolar ration to the chimeric nuclease.

An embodiment of the claimed invention is a composition of other chimeric nucleases containing different combinations of an I-TevI domain, a linker domain and an RNA-guided nuclease domain. In particular, the composition is directed to chimeric nucleases of SEQ ID NOs: 44-49.

In one aspect the nucleases described herein are chimeric nucleases formed from two different nucleases. The chimeric nucleases are useful for the ex vivo gene editing applications described herein and for in vivo applications.

In one aspect described herein is a chimeric nuclease comprising a modified I-TevI nuclease domain and/or I-TevI linker domain, a CasX polypeptide, and a guide RNA, wherein said chimeric nuclease optionally comprises a nuclear localization signal. In certain embodiments, the CasX polypeptide is from Planctomycetes bacterium. In certain embodiments, the CasX polypeptide is from Deltaproteobacteria. In certain embodiments, the modified I-TevI nuclease domain and/or the I-TevI linker domain comprises an amino acid modification at an amino acid corresponding to V117 of an unmodified I-TevI nuclease set forth in SEQ ID NO: 53. In certain embodiments, the modified I-TevI nuclease domain and/or the I-TevI linker domain comprises an amino acid modification corresponding to V117F of an unmodified I-TevI nuclease set forth in SEQ ID NO: 53. In certain embodiments, the modified I-TevI nuclease domain and/or the I-TevI linker domain comprises an amino acid modification at an amino acid corresponding to V117, K135, and N140 of an unmodified I-TevI nuclease set forth in SEQ ID NO: 53. In certain embodiments, the modified I-TevI nuclease domain and/or the I-TevI linker domain linker comprises an amino acid modification corresponding to V117F, K135R, and N140S of an unmodified I-TevI nuclease set forth in SEQ ID NO: 53. In certain embodiments, the modified I-TevI nuclease domain and/or the linker comprises an amino acid modification at an amino acid corresponding to K135 and N140 of an unmodified I-TevI nuclease set forth in SEQ ID NO: 53. In certain embodiments, the modified I-TevI nuclease domain and/or the I-TevI linker domain comprises an amino acid modification corresponding to K135R and a N140S of an unmodified I-TevI nuclease set forth in SEQ ID NO: 53. In certain embodiments, the modified I-TevI nuclease domain and/or the I-TevI linker domain comprises an amino acid modification at an amino acid corresponding to K26, T95, and aQ158 of an unmodified I-TevI nuclease set forth in SEQ ID NO: 53. In certain embodiments, the modified I-TevI nuclease domain and/or the linker comprises an amino acid modification corresponding to K26R, T95S, and Q158R of an unmodified I-TevI nuclease set forth in SEQ ID NO: 53. In certain embodiments, the modified I-TevI nuclease domain and/or the I-TevI linker domain comprises an amino acid sequence at least about 85%, 90%, 95%, 97%, 98%, 99% identical, or is identical to SEQ ID NO: 53. In certain embodiments, the CasX polypeptide comprises an amino acid sequence at least about 85%, 90%, 95%, 97%, 98%, 99% identical, or is identical to SEQ ID NO: 52. In certain embodiments, the Cas12 polypeptide comprises a substitution selected from any one or more of R11K, R12K, V145, K15A, S17N. N18A, A22V, G23S, T25S, P38D, K41K, E42K, N46K, L47R, N53V, I54M, P57V, T61N, S62A, R63A, A64N, E75K, H82Q, Q89K, P1045, N106K, 1113K, N199K, S124T, S125A, C133G, Y137F, N145S, D146E, H151Y, S161A, R165K, N177S, L180A, R202K, N205T, G215A, C219Y, V236I, T241S, L248I, I254V, 5269G, 1290V, E291D, V297I, Q299R, 1314L, E318D, Q323L, L333V, E359D, D360M, K362R, Q366S, N367G, L368V, A369T, G370A, Y371E, H404Y, H409Y, G410A, E411G, Y417F, V428I, E429A, S432T, K433S, L437R, S443A, A451V, I464L, A470M, 1502V, L503V, I531L, G537K, L540I, N553S, I559L, S563G, V571L, N579Q, H589T, 5607L, L6081, L620I, R623K, R624K, L644V, S646P, M652V, I657V, R679E, L684S, N686G, H689D, 5696G, T702A, T737S, L742F, Y744H, Q748H, M751V, I753V, A771T, R777K, P792T, S818T, R823G, V824M, E826V, K827R, A832S, T833D, M836A, I839L, G841N, V846A, N860T, V862E, D864E, V867A, V877G, S883K, 5889R, G890D, S894F, K908Q, N913D, F916H, T918V, R936N, Q938N, Y940F, K942S, S963A, R966K, K967R, or K968R. In certain embodiments, said chimeric nuclease comprises a nuclear localization signal. In certain embodiments, the nuclear localization signal comprises an SV40 nuclear localization signal comprising the amino acid sequence SEQ ID NO: 55 (PKKKRKV). In certain embodiments, the nuclear localization signal comprises a Nucleoplasmin nuclear localization signal comprising the amino acid sequence SEQ ID NO: 56 (KRPAATKKAGQAKKKK). In certain embodiments, the modified I-TevI linker domain comprises an amino acid sequence at least about 85%, 90%, 95%, 97%, 98%, 99% identical to SEQ ID NO: 57. In certain embodiments, the modified I-TevI nuclease domain comprises an amino acid sequence at least about 85%, 90%, 95%, 97%, 98%, 99% identical to SEQ ID NO: 58.

In one aspect described herein is a chimeric nuclease comprising a modified I-TevI nuclease domain and/or I-TevI linker domain, a Cas12 polypeptide, and a guide RNA, wherein said chimeric nuclease comprises a nuclear localization signal. In certain embodiments, the Cas12 polypeptide is from Acidaminococcus sp. BV3L6. In certain embodiments, the modified I-TevI nuclease domain and/or the I-TevI linker domain comprises an amino acid modification at an amino acid corresponding to V117 of an unmodified I-TevI nuclease set forth in SEQ ID NO: 53. In certain embodiments, the modified I-TevI nuclease domain and/or the I-TevI linker domain comprises an amino acid modification corresponding to V117F of an unmodified I-TevI nuclease set forth in SEQ ID NO: 53. In certain embodiments, the modified I-TevI nuclease domain and/or the I-TevI linker domain comprises an amino acid modification at an amino acid corresponding to V117, K135, and N140 of an unmodified I-TevI nuclease set forth in SEQ ID NO: 53. In certain embodiments, the modified I-TevI nuclease domain and/or the I-TevI linker domain linker comprises an amino acid modification corresponding to V117F, K135R, and N140S of an unmodified I-TevI nuclease set forth in SEQ ID NO: 53. In certain embodiments, the modified I-TevI nuclease domain and/or the linker comprises an amino acid modification at an amino acid corresponding to K135 and N140 of an unmodified I-TevI nuclease set forth in SEQ ID NO: 53. In certain embodiments, the modified I-TevI nuclease domain and/or the I-TevI linker domain comprises an amino acid modification corresponding to K135R and a N140S of an unmodified I-TevI nuclease set forth in SEQ ID NO: 53. In certain embodiments, the modified I-TevI nuclease domain and/or the I-TevI linker domain comprises an amino acid modification at an amino acid corresponding to K26, T95, and a Q158 of an unmodified I-TevI nuclease set forth in SEQ ID NO: 53. In certain embodiments, the modified I-TevI nuclease domain and/or the linker comprises an amino acid modification corresponding to K26R, T95S, and Q158R of an unmodified I-TevI nuclease set forth in SEQ ID NO: 53. In certain embodiments, the modified I-TevI nuclease domain and/or the I-TevI linker domain comprises an amino acid sequence at least about 85%, 90%, 95%, 97%, 98%, 99% identical, or is identical to SEQ ID NO: 53. In certain embodiments, the Cas12 polypeptide comprises an amino acid sequence at least about 85%, 90%, 95%, 97%, 98%, 99% identical, or is identical to SEQ ID NO: 51. In certain embodiments, the Cas12 polypeptide comprises a substitution selected from any one or more of T1S, Q2N, E4S, G5E, N8H, L9K, K28E, H29N, 130L, Q31T, E32A, Q33Y, F35M, I36V, E37N, E38D, A41L, N435, D44E, H45N, E48K, I52V, R55K, T59Y, Y60F, A61I, D62E, Q63E, C64T, Q66K, L67H, Q69A, L70I, N74P, S76Y, A77K, D80T, S81A, Y82F, E85D, E88L, T90N, R91N, N92T, A93N, I95R, E97I, A99D, T100N, Y101C, N103K, A1045, H106A, D107G, I110E, R112K, T113V, D114P, R159K, S169V, S185A, A1875, I192L, D195E, K2011, T212K, R218N, N223T, I228T, 5233G, I236L, E237D, V239I, F242V, Q249C, Y257F, V279T, I284V, F305Y, N3135, 5324N, I329L, S331A, T337E, L338K, L345I, E349Q, 5357L, I358A, N386D, I393V, L396A, 1400L, 5403N, V408I, Q409E, G427D, K428D, Q436A, L442I, S468V, Q469L, S472A, L473V, L479T, E487D, S488D, A497V, L510I, A516V, K522Q, Q535S, M536N, S541D, V545E, K549Q, N550Q, G552C, V557E, N559E, S586N, Y596Q, A601S, 1604L, A613D, S628N, E637T, A657D, K660R, G663N, Q665K, C673H, L683V, L697V, A711G, L717F, Q723E, A733L, E735D, Y740F, K751E, K756A, G766A, I778V, R793P, L844F, I858V, 5865T, I874L, H898N, 1903V, I916A, L931F, K941N, N945Q, V951I, S958T, V959A, D965E, I938V, H984Q, A10095, C1024Y, G10375, T1049E, G1055R, T1056N, Y1068F, L1075A, V1083R, K1085G, L1097I, H1104K, D1106N, D1111N, L1122K, A1134D, V1138I, D1147A, V1160E, P1161F, R1171Q, R1173E, Y1176L, N1205T, D1207N, 51220L, V1221T, A1230E, N1237S, L12431, M1259K, Q1274L, G1291A, Q1295N, A1299N, or L1304K. In certain embodiments, said chimeric nuclease comprises a nuclear localization signal. In certain embodiments, the nuclear localization signal comprises an SV40 nuclear localization signal comprising the amino acid sequence SEQ ID NO: 55 (PKKKRKV). In certain embodiments, the nuclear localization signal comprises a Nucleoplasmin nuclear localization signal comprising the amino acid sequence SEQ ID NO: 56 (KRPAATKKAGQAKKKK). In certain embodiments, the modified I-TevI linker domain comprises an amino acid sequence at least about 85%, 90%, 95%, 97%, 98%, 99% identical to SEQ ID NO: 57. In certain embodiments, the modified I-TevI nuclease domain comprises an amino acid sequence at least about 85%, 90%, 95%, 97%, 98%, 99% identical to SEQ ID NO: 58.

In one aspect described herein is a chimeric nuclease comprising a modified I-TevI nuclease domain and/or I-TevI linker domain, a modified Cas9 polypeptide, and a guide RNA, wherein the modified I-TevI nuclease domain and/or I-TevI linker domain comprises an amino acid modification corresponding to K26R, a T95S, and a Q158R of the unmodified I-TevI nuclease set forth in SEQ ID NO: 53, and the modified Cas9 polypeptide comprises an amino acid modification corresponding to D10E or of the unmodified Cas9 set forth in SEQ ID NO: 54. In certain embodiments, the modified I-TevI nuclease domain and/or I-TevI linker domain comprises an amino acid sequence at least about 80%, 90%, 95%, 97%, 98%, 99% identical, or is identical to SEQ ID NO: 53. In certain embodiments, said chimeric nuclease comprises a nuclear localization signal. In certain embodiments, the Cas9 polypeptide is from Staphylococcus aureus. In certain embodiments, the nuclear localization signal comprises an SV40 nuclear localization signal comprising the amino acid sequence SEQ ID NO: 55 (PKKKRKV). In certain embodiments, the nuclear localization signal comprises a Nucleoplasmin nuclear localization signal comprising the amino acid sequence SEQ ID NO: 56 (KRPAATKKAGQAKKKK). In certain embodiments, the modified I-TevI linker domain comprises an amino acid sequence at least about 85%, 90%, 95%, 97%, 98%, 99% identical to SEQ ID NO: 57. In certain embodiments, the modified I-TevI nuclease domain comprises an amino acid sequence at least about 85%, 90%, 95%, 97%, 98%, 99% identical to SEQ ID NO: 58.

The chimeric nucleases described herein can comprise a functional fragment of a Cas12, Cas9, CasX or an I-TevI nuclease described herein. Such functional fragments may comprise one or more of a n N-terminal deletion, a C-terminal deletion, or an intervening deletion preserving the N- and C-termini of the nuclease. Such functional fragments of the I-TevI nuclease may also comprise one or more of amino acids 1-93 of the I-TevI nuclease domain, amino acids 94-150 of the I-TevI linker fragment, amino acids 151-170 of a zinc-finger domain, or amino acids 171-180 of the artificial linker domain. Amino acid R28 of the I-TevI nuclease domain is required for DNA cleavage. Amino acids C152, C154, C165 and C168 of the zinc-finger domain are required for efficient DNA cleavage by the I-TevI nuclease domain. Such function fragments of the SaCas9 domain may also comprise one or more of amino acids. Certain functional fragments (with reference to SEQ ID NO: 54 are described herein. 1-39 and amino acids 434-1052 of a nuclease lobe, amino acids 41-424 of a recognition lobe. A functional fragment of the recognition domain includes amino acids 41-72 which form a bridge helix loop to connect to the N-terminal nuclease lobe. Amino acids 425-433 comprise a linker loop domain to connect the recognition loop and C-terminal nuclease lobe. The nuclease lobe further comprises the functional fragments of one or more of amino acids 1-39 of a RuvC-I domain, amino acids 434-479 of a RuvC-II domain, amino acids 649-773 of a RuvC-III domain, amino acids 519-627 of am HNH domain, amino acids 787-908 of a wedge (WED) domain, amino acids 909-1052 of a PAM-interacting (PI) domain. The PAM-interacting domain further comprises the functional fragments of amino acids 909-967 of a Topoisomerase-homology (TOPO) domain and amino acids 968-1052 of a C-terminal domain. The HNH domain is connected to the RuvC-II domain by amino acids 480-518 of the L1 linker domain and to the RuvC-III domain by amino acids 628-648 of the L2 linker domain. The RuvC-III and WED domains are connected by amino acids 774-786 of the phosphate lock loop domain. Amino acids D10, H557 and N580 of the saCas9 domain are required for DNA cleaving.

### Nucleases

In some embodiments, the composition of the modified TevSaCas9 nuclease contains different combinations of the I-TevI, SaCas9 domain and guide RNA as described herein.

In one aspect, described herein, is a chimeric nuclease comprising a modified I-TevI nuclease domain and/or I-TevI linker domain, a CasX polypeptide, and a guide RNA, wherein said chimeric nuclease optionally comprises a nuclear localization signal. In certain embodiments, the modified I-TevI nuclease domain and/or the I-TevI linker domain comprises a modification at one or more amino acid residues corresponding to K26, T95, V117, K135, N140, and Q158 of the unmodified I-TevI nuclease set forth in SEQ ID NO: 53. In certain embodiments, the modified I-TevI nuclease domain and/or the I-TevI linker domain comprises an amino acid modification at an amino acid corresponding to V117 of an unmodified I-TevI nuclease set forth in SEQ ID NO: 53. In certain embodiments, the modified I-TevI nuclease domain and/or the I-TevI linker domain comprises an amino acid modification corresponding to V117F of an unmodified I-TevI nuclease set forth in SEQ ID NO: 53. In certain embodiments, the modified I-TevI nuclease domain and/or the I-TevI linker domain comprises an amino acid modification at an amino acid corresponding to V117, K135, and N140 of an unmodified I-TevI nuclease set forth in SEQ ID NO: 53. In certain embodiments, the modified I-TevI nuclease domain and/or the I-TevI linker domain linker comprises an amino acid modification corresponding to V117F, K135R, and N140S of an unmodified I-TevI nuclease set forth in SEQ ID NO: 53. In certain embodiments, the modified I-TevI nuclease domain and/or the linker comprises an amino acid modification at an amino acid corresponding to K135 and N140 of an unmodified I-TevI nuclease set forth in SEQ ID NO: 53. In certain embodiments, the modified I-TevI nuclease domain and/or the I-TevI linker domain comprises an amino acid modification corresponding to K135R and a N140S of an unmodified I-TevI nuclease set forth in SEQ ID NO: 53. In certain embodiments, the modified I-TevI nuclease domain and/or the I-TevI linker domain comprises an amino acid modification at an amino acid corresponding to K26, T95, and a Q158 of an unmodified I-TevI nuclease set forth in SEQ ID NO: 53. In certain embodiments, the modified I-TevI nuclease domain and/or the linker comprises an amino acid modification corresponding to K26R, T95S, and Q158R of an unmodified I-TevI nuclease set forth in SEQ ID NO: 53. In certain embodiments, the modified I-TevI nuclease domain and/or the I-TevI linker domain comprises an amino acid sequence at least about 85%, 90%, 95%, 97%, 98%, 99% identical, or is identical to SEQ ID NO: 53. In certain embodiments, the CasX polypeptide comprises an amino acid sequence at least about 85%, 90%, 95%, 97%, 98%, 99% identical, or is identical to SEQ ID NO: 52. In certain embodiments, the Cas12 polypeptide comprises a substitution selected from any one or more of R11K, R12K, V145, K15A, S17N. N18A, A22V, G23S, T25S, P38D, K41K, E42K, N46K, L47R, N53V, I54M, P57V, T61N, S62A, R63A, A64N, E75K, H82Q, Q89K, P1045, N106K, 1113K, N199K, S124T, S125A, C133G, Y137F, N145S, D146E, H151Y, S161A, R165K, N177S, L180A, R202K, N205T, G215A, C219Y, V236I, T241S, L248I, I254V, S269G, 1290V, E291D, V297I, Q299R, 1314L, E318D, Q323L, L333V, E359D, D360M, K362R, Q366S, N367G, L368V, A369T, G370A, Y371E, H404Y, H409Y, G410A, E411G, Y417F, V428I, E429A, S432T, K433S, L437R, S443A, A451V, I464L, A470M, 1502V, L503V, I531L, G537K, L540I, N553S, I559L, S563G, V571L, N579Q, H589T, 5607L, L6081, L620I, R623K, R624K, L644V, S646P, M652V, I657V, R679E, L684S, N686G, H689D, S696G, T702A, T737S, L742F, Y744H, Q748H, M751V, I753V, A771T, R777K, P792T, S818T, R823G, V824M, E826V, K827R, A832S, T833D, M836A, I839L, G841N, V846A, N860T, V862E, D864E, V867A, V877G, S883K, S889R, G890D, S894F, K908Q, N913D, F916H, T918V, R936N, Q938N, Y940F, K942S, S963A, R966K, K967R, or K968R. In certain embodiments, the CasX polypeptide is from Planctomycetes bacterium. In certain embodiments, the CasX polypeptide is from Deltaproteobacteria.

In one aspect, described herein, is a chimeric nuclease comprising a modified I-TevI nuclease domain and/or I-TevI linker domain, a Cas12 polypeptide, and a guide RNA, wherein said chimeric nuclease comprises a nuclear localization signal. In certain embodiments, the modified I-TevI nuclease domain and/or the I-TevI linker domain comprises a modification at one or more amino acid residues corresponding to K26, T95, V117, K135, N140, and Q158 of the unmodified I-TevI nuclease set forth in SEQ ID NO: 53. In certain embodiments, the modified I-TevI nuclease domain and/or the I-TevI linker domain comprises an amino acid modification at an amino acid corresponding to V117 of an unmodified I-TevI nuclease set forth in SEQ ID NO: 53. In certain embodiments, the modified I-TevI nuclease domain and/or the I-TevI linker domain comprises an amino acid modification corresponding to V117F of an unmodified I-TevI nuclease set forth in SEQ ID NO: 53. In certain embodiments, the modified I-TevI nuclease domain and/or the I-TevI linker domain comprises an amino acid modification at an amino acid corresponding to V117, K135, and N140 of an unmodified I-TevI nuclease set forth in SEQ ID NO: 53. In certain embodiments, the modified I-TevI nuclease domain and/or the I-TevI linker domain linker comprises an amino acid modification corresponding to V117F, K135R, and N140S of an unmodified I-TevI nuclease set forth in SEQ ID NO: 53. In certain embodiments, the modified I-TevI nuclease domain and/or the linker comprises an amino acid modification at an amino acid corresponding to K135 and N140 of an unmodified I-TevI nuclease set forth in SEQ ID NO: 53. In certain embodiments, the modified I-TevI nuclease domain and/or the I-TevI linker domain comprises an amino acid modification corresponding to K135R and a N140S of an unmodified I-TevI nuclease set forth in SEQ ID NO: 53. In certain embodiments, the modified I-TevI nuclease domain and/or the I-TevI linker domain comprises an amino acid modification at an amino acid corresponding to K26, T95, and a Q158 of an unmodified I-TevI nuclease set forth in SEQ ID NO: 53. In certain embodiments, the modified I-TevI nuclease domain and/or the linker comprises an amino acid modification corresponding to K26R, T95S, and Q158R of an unmodified I-TevI nuclease set forth in SEQ ID NO: 53. In certain embodiments, the modified I-TevI nuclease domain and/or the I-TevI linker domain comprises an amino acid sequence at least about 85%, 90%, 95%, 97%, 98%, 99% identical, or is identical to SEQ ID NO: 53. In certain embodiments, the Cas12 polypeptide is from Acidaminococcus sp. BV3L6. In certain embodiments, the Cas12 polypeptide comprises an amino acid sequence at least about 85%, 90%, 95%, 97%, 98%, 99% identical, or is identical to SEQ ID NO: 51. In certain embodiments, the Cas12 polypeptide comprises a substitution selected from any one or more of T1S, Q2N, E45, GSE, N8H, L9K, K28E, H29N, 130L, Q31T, E32A, Q33Y, F35M, I36V, E37N, E38D, A41L, N435, D44E, H45N, E48K, I52V, R55K, T59Y, Y60F, A61I, D62E, Q63E, C64T, Q66K, L67H, Q69A, L70I, N74P, S76Y, A77K, D80T, S81A, Y82F, E85D, E88L, T90N, R91N, N92T, A93N, I95R, E97I, A99D, T100N, Y101C, N103K, A1045, H106A, D107G, I110E, R112K, T113V, D114P, R159K, S169V, S185A, A1875, I192L, D195E, K2011, T212K, R218N, N223T, I228T, 5233G, I236L, E237D, V239I, F242V, Q249C, Y257F, V279T, I284V, F305Y, N3135, 5324N, I329L, S331A, T337E, L338K, L345I, E349Q, 5357L, I358A, N386D, I393V, L396A, 1400L, 5403N, V408I, Q409E, G427D, K428D, Q436A, L442I, S468V, Q469L, S472A, L473V, L479T, E487D, S488D, A497V, L510I, A516V, K522Q, Q535S, M536N, S541D, V545E, K549Q, N550Q, G552C, V557E, N559E, S586N, Y596Q, A601S, 1604L, A613D, S628N, E637T, A657D, K660R, G663N, Q665K, C673H, L683V, L697V, A711G, L717F, Q723E, A733L, E735D, Y740F, K751E, K756A, G766A, I778V, R793P, L844F, I858V, 5865T, I874L, H898N, 1903V, I916A, L931F, K941N, N945Q, V951I, S958T, V959A, D965E, I938V, H984Q, A10095, C1024Y, G10375, T1049E, G1055R, T1056N, Y1068F, L1075A, V1083R, K1085G, L1097I, H1104K, D1106N, D1111N, L1122K, A1134D, V1138I, D1147A, V1160E, P1161F, R1171Q, R1173E, Y1176L, N1205T, D1207N, 51220L, V1221T, A1230E, N1237S, L12431, M1259K, Q1274L, G1291A, Q1295N, A1299N, or L1304K. In certain embodiments, the chimeric nuclease has cleavage activity against an I-TevI recognition site in double-stranded DNA.

In one aspect, described herein, is a chimeric nuclease comprising a modified I-TevI nuclease domain and/or I-TevI linker domain, a modified Cas9 polypeptide, and a guide RNA, wherein the modified I-TevI nuclease domain and/or I-TevI linker domain comprises an amino acid modification corresponding to K26R, a T95S, and a Q158R of the unmodified I-TevI nuclease set forth in SEQ ID NO: 53, and the modified Cas9 polypeptide comprises an amino acid modification corresponding to D10E or of the unmodified Cas9 set forth in SEQ ID NO: 54. In certain embodiments, the modified I-TevI nuclease domain and/or I-TevI linker domain comprises an amino acid sequence at least about 80%, 90%, 95%, 97%, 98%, 99% identical, or is identical to SEQ ID NO: 53. In certain embodiments, said chimeric nuclease comprises a nuclear localization signal. In certain embodiments, the Cas9 polypeptide is from Staphylococcus aureus. In certain embodiments, the nuclear localization signal comprises an SV40 nuclear localization signal comprising the amino acid sequence SEQ ID NO: 55 (PKKKRKV). In certain embodiments, the nuclear localization signal comprises a Nucleoplasmin nuclear localization signal comprising the amino acid sequence SEQ ID NO: 56 (KRPAATKKAGQAKKKK). In certain embodiments, the modified I-TevI linker domain comprises an amino acid sequence at least about 85%, 90%, 95%, 97%, 98%, 99% identical to SEQ ID NO: 57. In certain embodiments, the modified I-TevI nuclease domain comprises an amino acid sequence at least about 85%, 90%, 95%, 97%, 98%, 99% identical to SEQ ID NO: 58. In certain embodiments, the chimeric nuclease has cleavage activity against an I-TevI recognition site in double-stranded DNA.

### I-TevI Domain

The sequence in SEQ ID NO: 8 is wild-type version of I-TevI except for a glycine substation at position 2 that is known to help increase protein stability and prevent N-terminal degradation. With respect to specific substitutions referred to herein, the numbering corresponds to the wild-type version of the protein lacking the glycine stabilization. Thus, in the stabilized version of I-TevI the lysine at position 27 of SEQ ID NO 8 is referred to as K26 corresponding to the wild-type position without the glycine at position 2. Other I-TevI mutations are referred to accordingly. There are several I-TevI substitutions to the I-TevI domain known to have little effect on I-TevI nuclease activity, including T11V, V16I, N14G, E25D, K26R, R27A, E36S, K37N, G38N, C39V, S41H, L45F, F49Y, 160V, E81I (referring to the wild-type version of the I-TevI). Nuclease activity of I-TevI can be assayed for by mixing a chimeric nuclease containing the I-TevI domain with linear DNA containing a known I-TevI target and resolving the products of the cleavage reaction on an agarose gel. Products of the predicted size will be present if the I-TevI nuclease is active. In certain embodiments, substitutions to I-TevI allow for at least 50%, 60%, 70%, 80%, 90% or more of the I-TevI nuclease activity to be preserved compared to a wild-type unmodified I-TevI.

Other versions of the I-TevI nuclease domain might contain different combinations of mutations to alter the site targeted by the I-TevI domain or the activity of the I-TevI domain, including mutations that alter the sequence recognized by I-TevI, such as K26 and/or C39. Other versions of the nuclease might substitute the I-TevI domain with other GIY-YIG nuclease domains, such as I-BmoI, Eco29kI, etc. Some versions of I-TevI do not contain Met1 as a result of processing when expressed in E. coli.

In certain embodiments, the modified I-TevI nuclease domain comprises an amino acid sequence that is at least 85%, 90%, 95%, 97%, 98%, 99% identical to SEQ ID NO: 8. In certain embodiments, the modified I-TevI nuclease domain comprises a substitution selected from any one or more of T11V, V16I, N14G, E25D, K26R, R27A, E36S, K37N, G38N, C39V, S41H, L45F, F49Y, I60V, and E81I. In certain embodiments, the modified I-TevI nuclease domain comprises a K26R substitution. In certain embodiments, the modified I-TevI nuclease domain comprises SEQ ID NO: 8.

### Linker Domain

In certain embodiments, the I-TevI nuclease domain is joined to the saCas9 by a linker. The linker may comprise the I-TevI linker (amino acids 93-150 or I-TevI). The linker may alternatively or further comprise a flexible amino acid linker comprising from 10 to 100 amino acids. Such linkers can be unstructured or comprise a Gly-Ser linker.

In certain embodiments, the linker comprises an amino acid sequence that is at least 85%, 90%, 95%, 97%, 98%, 99% identical to any one of SEQ ID NOs: 9-14, or 59. In certain embodiments, the linker comprises a substitution selected from any one or more of T95S, S101Y, A119D, K120N, K135N, K135R, P126S, D127K, N140S, T147I, Q158R, A161V, or S165G. In certain embodiments, the linker comprises a substitution selected from any one or more of T95S, V117F, K135R, N140S, or Q158R. In certain embodiments, the linker is a peptide selected from any one of SEQ ID NOs: 9-14, or 59.

### SaCas9 Domain

Other versions of the nuclease might substitute the SaCas9 domain with other non-naturally occurring SaCas9 or SpCas9 domains in SEQ ID NOs: 25-28. Other versions of the chimeric nuclease may substitute the SaCas9 domain with other nucleases including the chimeric nucleases TevCas12a of SEQ ID NOs: 44-48 or TevCasX of SEQ ID NO: 49. Further embodiments of the chimeric nuclease may substitute the SaCas9 domain for other Class 1 or Class 2 CRISPR-Cas proteins, CRISPR-Cas3, CRISPR-Cascade, Cas13d. Other versions of the nuclease may substitute the SaCas9 domain with other heterologous polypeptide sequences, including polypeptide sequences capable of binding nucleic acids, polypeptide sequences capable of binding other polypeptide sequences, polypeptide targeting sequences. Other heterologous sequences may include 1 to 10 other polypeptide domains.

In certain embodiments, the RNA-guided nuclease Staphylococcus aureus Cas9 domain comprises any one of SEQ ID NOs: 15, 16, 26, 27 or 28. In certain embodiments, the RNA-guided nuclease Staphylococcus aureus Cas9 domain comprises an amino acid sequence that is at least 85%, 90%, 95%, 97%, 98%, 99% identical, or is identical to any one of SEQ ID NOs: 15, 16, 26, 27 or 28. In certain embodiments, the RNA-guided nuclease Staphylococcus aureus Cas9 domain comprises a substitution selected from any one or more of T267A, L325F, V327I, D333G, A336S, I341L, E345D, D348N, K352E, S360A, T368A, N369E, N371E, S372P, E373K, K386T, N393R, H408N, N410S, 1414M, A415T, T438S, Y467F, N471K, D485E, M489F, E506K, R409K, T510E, N515K, Y518F, A539P, F550Y, N551H, S596A, T6021, A6115, I617V, T620K, G654E, N667D, R685K, K695Q, 1706V, K722T, A723T, K724N, M731T, F732V, K735Q, S739N, P741L, E742G, E746D, Q747D, I754D, T7551, H757R, K760Q, H761S, P778I, E781K, I783V, N784D, D785E, T786L, L787V, Y788H, K792E, D794T, T798R, L799I, V801I, N8035, L8041, N805K, G806N, D813G, K814E, L818I, 1819F, S822P, E824G, L841T, G847S, D848N, Y857H, V875I, I876V, N884K, A888V, L890R, D894G, D895H, P897L, V903I, G920D, F924L, N929Y, E936D, N937G, V941I, N942D, S943L, C945A, E947K, K951R, L952Q, S956N, N957E, Q958K, A959S, N974D, G975K, V983A, N984S, N985D, D986G, I991V, V993L, M995F, I996V, T999N, Y1000K, R1001E, E1002D, L10041, E1005K, N1006M, M1007N, D1009L, K1010S, R1011T, P1012S, P1013F, 11015L, 11016R, A1020G, S1021K, Q1024K, K1027S, E1039K, H1045K, I0148M, or K1050M. In certain embodiments, the RNA-guided nuclease Staphylococcus aureus Cas9 domain comprise a D10E substitution. In certain embodiments, the modified I-TevI nuclease domain comprises SEQ ID NO: 8, the linker comprises any one of SEQ ID NOs: 9-14, or 59 and the RNA-guided nuclease Staphylococcus aureus Cas9 domain comprises any one of SEQ ID NOs: 15, 16, 26, 27 or 28. In certain embodiments, the modified I-TevI nuclease domain comprises an amino acid sequence that is at least 85%, 90%, 95%, 97%, 98%, 99% identical, or is identical to any one of SEQ ID NO: 8, the linker domain comprises an amino acid sequence that is at least 85%, 90%, 95%, 97%, 98%, 99% identical, or is identical to any one SEQ ID NOs: 9-14 and the RNA-guided nuclease Staphylococcus aureus Cas9 domain comprises an amino acid sequence that is at least 85%, 90%, 95%, 97%, 98%, 99% identical to any one SEQ ID NOs: 15, 16, 26, 27 or 28.

### Donor DNA and Therapeutic Cell Populations

The methods and techniques described herein are useful for the genetic modification of a cell of a population of cells ex vivo. Such ex vivo modification can be useful for the production of therapeutic cells. Such therapeutic modifications may comprise the removal or disruption of a genomic DNA sequence. Other therapeutic modifications may comprise the replacement or repair of a genomic DNA sequence. The donor DNAs described herein may be targeted to a specific site in the genome of a cell such as to replace or repair (i.e., correct one or more genetic mutations responsible for a disease) an existing gene or to a safe harbor site such as the AAVS1 site. The exogenous donor DNA may be configured for incorporation by homologous recombination. Such exogenous donor DNAs for incorporation by homologous recombination may comprise a first flanking homology region, an exogenous polynucleotide sequence of interest, and a second flanking homology region. Alternatively, the exogenous donor DNA may be inserted into a genomic location by incorporation into a genomic location at a single double strand break or a dual double stranded break with the aid of non-homologous end joining.

The therapeutic cells or population of therapeutic cells can be produced from pluripotent cells. In certain embodiments, the therapeutic cells or population of pluripotent cells comprise or consist of any one or more of murine, canine, feline, equine, porcine, ovine, bovine or human pluripotent cells. In certain embodiments, the therapeutic cells or population of therapeutic cells comprise or consist of pluripotent cells. The pluripotent cells may be isolated from an animal tissue, or induced using known methods to induce pluripotent stem cells.

The therapeutic cells or population of therapeutic cells can be produced from immune cells. In certain embodiments, the therapeutic cells or population of therapeutic cells comprise or consist of any one or more of murine, canine, feline, equine, porcine, ovine, bovine or human immune cells. In certain embodiments, the therapeutic cells or population of therapeutic cells comprise or consist of human immune cells.

The cells can be an isolated cell population of heterogeneous immune cells (e.g., peripheral blood mononuclear cells), or isolated and purified populations of specific immune cells such as lymphocytes, T cells, B cells, or NK cells. Such cell populations can be isolated to high purity greater than about 80%, 85%, 90%, or 95% by techniques known in the art such as magnet bead selection (positive or negative selection).

The therapeutic cells or population of therapeutic cells can be produced from mammalian hematopoietic stem cells. In certain embodiments, the therapeutic cells or population of therapeutic cells comprise or consist of any one or more of murine, canine, feline, equine, porcine, ovine, bovine or human hematopoietic stem cells. In certain embodiments, the therapeutic cells or population of therapeutic cells comprise or consist of human hematopoietic stem cells.

The therapeutic cells or population of therapeutic cells can be produced from mammalian mesenchymal stem cells. In certain embodiments, the therapeutic cells or population of therapeutic cells comprise or consist of any one or more of murine, canine, feline, equine, porcine, ovine, bovine or human mesenchymal stem cells. In certain embodiments, the therapeutic cells or population of therapeutic cells comprise or consist of mesenchymal stem cells.

In certain embodiments, the therapeutic cell is a CAR T cell. In certain embodiments, the therapeutic cell is a CAR NK cell. Therefor donor DNAs to be delivered with the chimeric nucleases described herein can encode a chimeric antigen receptor (CAR). CARs generally comprise a targeting moiety derived from an antibody, a transmembrane domain, and one or more intracellular singling domains that activate cytotoxic activity in a T cell or NK cell.

In certain embodiments, the therapeutic cell is a universal CAR T or a CAR T with reduced immunogenicity. Such reduced immunogenicity can be achieved by introducing one or more disruptions in a T cell receptor alpha chain, a T cell receptor beta chain, or an MHC gene (e.g., B2M, HLA-A, HLA-B, or HLA-C).

When a TevSaCas9 chimeric nuclease cleaves a double stranded DNA the Cas9 leaves a blunt end and the I-TevI leaves a two nucleotide 3′ overhang. Donor DNAs supplied may include a blunt end and a two nucleotide 3′ overhang configured to bind the created 3′ overhang in the TevSaCas9 cleaved site.

In one aspect the donor DNA comprises DNA sequences that are intended to be inserted into a genomic site that is not known to code for a functional gene. An example of such a site is AAVS1. It also comprises DNA sequences that are not found in the target genomic DNA; these sequences may code for useful genes and/or other DNA elements. In certain embodiments, the donor DNA comprises double-stranded DNA of the same length cleaved by the nuclease and also comprising complimentary DNA ends to those cleaved by TevSaCas9. In certain embodiments, the donor DNA comprises 5′ ends of the DNA that are phosphorylated. In certain embodiments, the donor DNA comprises circular double-strand DNA comprising an I-TevI target site and SaCas9 target site where the product cleaved from the double-strand DNA contains complimentary ends to those cleaved by TevSaCas9.

### Guide RNA

Other versions of the guide RNA might target the same region of DNA in the B2M, TRAC, TRBC, HLA-A, AVVS1 genes, but contain different sequences to account for genetic polymorphism in populations. Other versions of the guide RNA might target different sequences in the B2M, TRAC, TRBC, HLA-A, AVVS1. Other version of the guide RNA might target other sequences in a genome to retarget the nuclease to additional safe harbor sites such as hROSA26, and CCR5. Other versions might contain a mixture of guide RNAs to target multiple sequences within the same gene. Guide RNAs of the described invention may comprise a single strand comprising all necessary elements for activity (e.g., target binding and nuclease binding). Alternatively guide RNAs may comprise two or more non-covalently bound nucleic acids that forma single moiety due to base paring between the two or more nucleic acids. Other versions of the guide RNA might include different nucleobases for stability including, but not limited to, a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-propynyl uracil; a 5-propynyl cytosine; a 6-azo uracil; a 6-azo cytosine; a 6-azo thymine; a pseudouracil; a 4-thiouracil; an 8-haloadenin; an 8-aminoadenin; an 8-thioladenin; an 8-thioalkyladenin; an 8-hydroxyladenin; an 8-haloguanin; an 8-aminoguanin; an 8-thiolguanin; an 8-thioalkylguanin; an 8-hydroxylguanin; a 5-halouracil; a 5-bromouracil; a 5-trifluoromethyluracil; a 5-halocytosine; a 5-bromocytosine; a 5-trifluoromethylcytosine; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deazaguanosine; a 2-aminopyridine; a 2-pyridone; a 5-substituted pyrimidine; a 6-azapyrimidine; an N-2, N-6 or 0-6 substituted purine; a 2-aminopropyladenine; a 5-propynyluracil; and a 5-propynylcytosine. Other versions of guide RNA may include other nucleic acids such as bridged nucleic acids or locked nucleic acids.

The guide RNAs described herein can further comprise one or more of a non-natural internucleoside linkage, a nucleic acid mimetic, a modified sugar moiety, and a modified nucleobase. In certain embodiments, the non-natural internucleoside linkage comprises one or more of: a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a 3′-amino phosphoramidate, an aminoalkylphosphoramidate, a phosphorodiamidate, a thionophosphoramidate, a thionoalkylphosphonate, a thionoalkylphosphotriester, a selenophosphate, and a boranophosphate. In certain embodiments, the nucleic acid mimetic comprises one or more of a peptide nucleic acid (PNA), morpholino nucleic acid, cyclohexenyl nucleic acid (CeNAs), or a locked nucleic acid (LNA). IN certain embodiments, the modified sugar moiety comprises one or more of 2′-O-(2-methoxyethyl), 2′-dimethylaminooxyethoxy, 2′-dimethylaminoethoxyethoxy, 2′-O-methyl, and 2′-fluoro. In certain embodiments the modified nucleobase comprises one or more of: a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-halouracil; a 5-halocytosine; a 5-propynyl uracil; a 5-propynyl cytosine; a 6-azo uracil; a 6-azo cytosine; a 6-azo thymine; a pseudouracil; a 4-thiouracil; an 8-halo; an 8-amino; an 8-thiol; an 8-thioalkyl; an 8-hydroxyl; a 5-halo; a 5-bromo; a 5-trifluoromethyl; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deaza-adenine; a 7-deazaguanosine; a 2-aminopyridine; a 2-pyridone; a 5-substituted pyrimidine; a 6-azapyrimidine; an N-2, N-6 or O-6 substituted purine; a 2-aminopropyladenine; a 5-propynyluracil; and a 5-propynylcytosine.

Composition of the donor DNA: Other versions of the double-stranded donor DNA as discussed above contain different 5′-end chemical modifications such as biotin. Other versions of the donor DNA might include stability modifications to the 2′ position of the ribose, including but not limited to 2′-fluoro, 2′-amino, and 2′-O-methyl. Other versions of the donor DNA may contain 3′-end modifications such as an inverted dT or biotin. Other versions of the donor DNA might include locked nucleic acids (LNAs) in which the 2′-O and 4′-C atoms of the ribose sugar are joined through a methylene bridge. Other versions of the double-stranded donor DNA might include circular plasmid DNA containing a TevSaCas9 target site in which cleavage with TevSaCas9 creates complimentary DNA ends to those in the genome target. The double stranded donor DNA may comprise a synthetic or amplified linear double stranded DNA. In certain embodiments the donor DNA is supplied using a viral vector such as an adeno-associated virus or lentivirus.

### Production of Chimeric Nucleases

Chimeric nucleases described herein may be produced in many ways including using an E. coli expression system as described in WO2020225719A1. Alternatively, the chimeric nucleases may be produced by the target cell to be modified by supplying one or more genetic vectors that directs expression and production of the nucleases in the target cell. Additionally, the vector may provide sequences to direct expression of guide RNAs to target the chimeric nuclease to particular genomic region.

An exemplary method for producing a genetically engineered cell as described herein is described below.

A population of cells is grown in a T flask to 70-90% confluency. The cells are harvested by centrifugation and resuspended to 1.0×10⁷ cells per milliliter (practical range 0.2-2×10⁷ cells per milliliter) in Buffer T (Invitrogen®, Carlsbad, California, US). Cells are electroporated with a nuclease described herein including those selected from SEQ ID NOs: 25-49 and formulated in a Tris(hydroxymethyl)aminomethane or phosphate buffered saline with a Neon® Transfection System (Thermo Fisher Scientific®, Waltham, Massachusetts, US) at 2000 volts (practical range 1100-2500), 20 milliseconds (practical range 10-30 milliseconds) and 1 pulse (practical range 1-4). Cells are recovered in RPMI 1640 with 0.3 g/L glutamine and 2 g/L glucose (Sigma-Aldrich®, Irvine, UK), 10% fetal bovine serum (Sigma-Aldrich®, Oakville, Ontario, CA), 2 mM L-glutamine, and 100 units penicillin and 0.1 mg streptomycin/mL (Sigma-Aldrich®, St. Louis, Missouri, US) for 24 hours. Dead cells are removed using a Dead Cell Removal Kit (Miltenyi Biotec®, Somerville, Massachusetts, US). Knockout efficiency is measured by amplifying the target genes by polymerase chain reaction and measuring the proportion of cells edited by targeted amplicon sequencing (GENEWIZ, South Plainfield, NJ, US). Amplicon sequencing is a method of targeted next generation sequencing that enables you to analyze genetic variation in specific genomic regions. This method uses PCR to create sequences of DNA called amplicons. Amplicons from different samples can be multiplexed, also called indexed or pooled, which involves adding a barcode (index) to samples so they can be identified. Before multiplexing, individual samples used for amplicon sequencing must be transformed into libraries by adding adapters and enriching target regions via PCR amplification. The adapters allows formation of indexed amplicons and allow the amplicons to adhere to the flow cell for sequencing. Amplicon sequencing is typically used for variant detection in a population of cells.

Methods of producing ex vivo cell therapies A method to manufacture modified cell therapies: Other methods to deliver the nuclease to the cell may be used, such as a lipid nanoparticle, polymer, viral vector or cell penetrating peptides. The chimeric nuclease or guide RNA may be delivered separately or in combination as DNA or RNA in either single-stranded or double-stranded form. Further, the chimeric nuclease may be delivered as RNA containing one or more of the following elements: a 5′ cap, a 5′ untranslated region, a coding sequence, a 3′ untranslated region and a poly adenine (poly-A) tail. The RNA might include different nucleobases for stability including, but not limited to, a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-propynyl uracil; a 5-propynyl cytosine; a 6-azo uracil; a 6-azo cytosine; a 6-azo thymine; a pseudouracil; a 4-thiouracil; an 8-haloadenin; an 8-aminoadenin; an 8-thioladenin; an 8-thioalkyladenin; an 8-hydroxyladenin; an 8-haloguanin; an 8-aminoguanin; an 8-thiolguanin; an 8-thioalkylguanin; an 8-hydroxylguanin; a 5-halouracil; a 5-bromouracil; a 5-trifluoromethyluracil; a 5-halocytosine; a 5-bromocytosine; a 5-trifluoromethylcytosine; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deazaguanosine; a 2-aminopyridine; a 2-pyridone; a 5-substituted pyrimidine; a 6-azapyrimidine; an N-2, N-6 or O-6 substituted purine; a 2-aminopropyladenine; a 5-propynyluracil; and a 5-propynylcytosine.

In another embodiment, the chimeric nuclease may be delivered as an integrating vector including, but not limited to retrovirus vectors, lentivirus vectors, transposon vectors, and adeno-associated virus vectors. The chimeric nuclease may also be delivered by other electroporation systems, including but not limited to a Nucleofector™ (Lonza, Basel, Switzerland), MaxCyte (Gaithersburg, MD) or CliniMACS® (Bergisch Gladbach, Germany).

The chimeric nucleases may further be included in a pharmaceutical composition comprising one or more of a pharmaceutically acceptable carrier, diluent, or excipient. The term “pharmaceutically acceptable excipient,” as used herein, refers to carriers and vehicles that are compatible with the active ingredient (for example, a compound of the invention) of a pharmaceutical composition of the invention (and preferably capable of stabilizing it) and not deleterious to the subject to be treated. For example, solubilizing agents that form specific, more soluble complexes with the compounds of the invention can be utilized as pharmaceutical excipients for delivery of the compounds. Suitable carriers and vehicles are known to those of extraordinary skill in the art. The term “excipient” as used herein will encompass all such carriers, adjuvants, diluents, solvents, or other inactive additives. Pharmaceutical formulations may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, glycerol, tetrahydrofuryl alcohol, and fatty acid esters of sorbitan, cyclodextrins, albumin, hyaluronic acid, chitosan and mixtures thereof. Polyethylene glycol (PEG) may be used to obtain desirable properties of solubility, stability, half-life and other pharmaceutically advantageous properties. Representative examples of stabilizing components include polysorbate 80, L-arginine, polyvinylpyrrolidone, trehalose, and combinations thereof. Other excipients that may be employed, such as solution binders or anti-oxidants include, but are not limited to, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C and xylitol.

### Certain Embodiments

Described are certain specific numbered embodiments of the disclosure
1. A polypeptide comprising a modified I-TevI nuclease domain, a linker and a modified RNA-guided nuclease domain.
2. A chimeric nuclease comprising a modified I-TevI nuclease domain, a linker, an RNA-guided nuclease Staphylococcus aureus Cas9 domain, and a guide RNA wherein said chimeric nuclease has two active nuclease sites.
3. The chimeric nuclease according to embodiment 2, wherein the modified I-TevI nuclease domain is SEQ ID NO: 8 or a fragment thereof.
4. The chimeric nuclease according to embodiment 2, wherein the linker is a peptide selected from any one of SEQ ID NOs: 9-14 or a fragment thereof
5. The chimeric nuclease according to embodiment 2, wherein the RNA-guided nuclease Staphylococcus aureus Cas9 domain is any one of SEQ ID NOs: 15, 16, 26, 27 or 28 or a fragment thereof
6. The chimeric nuclease according to embodiment 2, wherein the modified I-TevI nuclease domain is SEQ ID NO: 8 or a fragment thereof, the linker is any one of SEQ ID NOs: 9-14 or a fragment thereof and the RNA-guided nuclease Staphylococcus aureus Cas9 domain is any one of SEQ ID NOs: 15, 16, 26, 27 or 28 or a fragment thereof
7. A method of disrupting a targeted mammalian gene comprising the step of administering the chimeric nuclease according to embodiment 6 wherein the genomic DNA of the target cell is modified to disrupt the expression of endogenous genes.
8. The method of disrupting a targeted mammalian gene according to embodiment 7, wherein all of the targeted gene is disrupted.
9. The method of disrupting a targeted mammalian gene according to embodiment 7, wherein part of the targeted gene is disrupted.
10. A method to edit the genetic makeup of a cell comprising the step of administering the chimeric nuclease according to embodiment 6.
11. The method to edit the genetic makeup of a cell according to embodiment 10, wherein said chimeric nuclease is administered in the presence of exogenous DNA.
12. The method to edit the genetic makeup of a cell according to embodiment 10, wherein said chimeric nuclease is administered to the nucleus of a target cell.
13. The method to edit the genetic makeup of a cell according to embodiment 12, wherein one or more nuclear-localization sequences are used to administer the chimeric nuclease.
14. The method according to embodiment 10, wherein said chimeric nuclease is delivered to genomic DNA.
15. The method according to embodiment 14, wherein said chimeric nuclease is delivered to the genomic DNA of target cells.
16. The method according to embodiment 15, wherein the target cells are immune cells.
17. The method according to embodiment 16, wherein the immune cells are T cells.
18. The method according to embodiment 15, wherein the target cells are pluripotent cells.
19. The method according to embodiment 18, wherein the pluripotent cells are induced pluripotent cells.
20. The method according to embodiment 15, wherein said chimeric nuclease is delivered to the genomic DNA of target cells using a DNA expression cassette.
21. The method according to embodiment 10, wherein said method does not use a viral vector.
22. The method according to embodiment 15, wherein said chimeric nuclease is administered to target cells that are isolated.
23. The method according to embodiment 22, wherein said isolated target cells are isolated in culture ex vivo.
24. The method according to embodiment 23, wherein said isolated target cells are exogenous donor cells.
25. The method according to embodiment 23, wherein said ex vivo culture of isolated target cells together with the chimeric nuclease is subjected to electroporation.
26. The method according to embodiment 25, wherein the electroporation is applied at a current of 1000 to 2500 V.
27. The method according to embodiment 20, wherein said chimeric nuclease permeates said isolated target cells ex vivo following the electroporation.
28. The method according to embodiment 14, wherein said cells are isolated from the group consisting of animal cells, bacteria cells, insect cells, and plant cells.
29. A method to delete defined lengths of genomic DNA comprising the step of delivering the chimeric nuclease according to embodiment 6 to a target cell.
30. The method according to embodiment 29, wherein the modified genomic DNA disrupts the expression of endogenous genes by knocking a gene out-of-frame.
31. A method to insert one or more defined lengths of a select exogenous DNA into the DNA of target cells comprising the step of delivering the chimeric nuclease according to embodiment 6 to a target cell.
32. The method according to embodiment 31, wherein the administered chimeric nuclease inserts one or more defined lengths of a select exogenous DNA into the DNA of target cells in the presence of exogenous donor DNA.
33. The method according to embodiment 32, wherein said exogenous donor DNA is inserted into the DNA of the target cells.
34. The method according to embodiment 31, wherein said defined lengths of select DNA comprise one or more functional genes or one or more fragments thereof
35. A method to delete one or more defined lengths of a select DNA in the DNA of target cells comprising the step of delivering the chimeric nuclease according to embodiment 6 to a target cell.
36. The method according to 35, wherein the chimeric nuclease is administered in the absence of exogenous donor DNA.
37. The methods according to embodiments 32 or 35, wherein the chimeric nuclease modifies the genomic DNA of the target cells to express one or more exogenous genes.
38. A method to delete one or more defined lengths of a select genomic DNA in target cells and to insert one or more defined lengths of a select exogenous DNA in the DNA of target cells comprising the step of delivering the chimeric nuclease according to embodiment 6 to target cells, wherein said select exogenous DNA is either inserted in the deleted section of the modified genomic DNA or is inserted at a different position in the select genomic DNA.
39. The methods according to embodiments 32, 35 or 38, wherein said exogenous gene expresses a chimeric antigen receptor.
40. The method according to embodiment 10, wherein said chimeric nuclease targets two independent sites on the genomic DNA of targeted cells.
41. The method according to embodiment 40, wherein the genomic DNA of the target cells is cleaved at one of the independent sites on the genomic DNA of the targeted cells.
42. The method according to embodiment 40, wherein the genomic DNA of the target cells is cleaved at both independent sites on the genomic DNA of the targeted cells creating two cleaved sites in the genomic DNA.
43. The method according to embodiments 41 or 42, wherein the cleaved genomic DNA are 30-36 bases in length.
44. The method according to embodiment 42, wherein the cleaving occurs in the presence of exogenous donor DNA.
45. The method according to embodiments 44, wherein the exogenous donor DNA is inserted between the two cleaved sites.
46. The method according to embodiments 45, wherein the entire exogenous donor DNA is inserted between the two cleaved sites.
47. The method according to embodiments 45, wherein a portion of the exogenous donor DNA is inserted between the two cleaved sites.
48. The method according to embodiments 45-47, wherein the exogenous donor DNA is inserted in a specific orientation between the two cleaved sites.
49. The methods according to embodiment 48, wherein the exogenous donor DNA is bound to the genomic DNA by direct ligation, microhomology end joining or non-homologous end joining.
50. The method according to and one of embodiments 41 or 42, wherein the guide RNA of said chimeric nuclease is either SEQ ID NO:17 or SEQ ID NO:18, or a fragment thereof; wherein said chimeric nuclease targets and cleaves the B2M gene.
51. The method according to embodiment 50, wherein said chimeric nuclease generates an out-of-frame mutation of the B2M gene.
52. The method according to embodiment 51, wherein said out-of-frame mutations inhibits proper B2M protein generation or function.
53. The method according to any one of embodiments 41 or 42, wherein the guide RNA of said chimeric nuclease is SEQ ID NO:19, or a fragment thereof; wherein said chimeric nuclease targets and cleaves the TRAC1 gene.
54. The method according to embodiment 53, wherein said chimeric nuclease generates an out-of-frame mutation of the TRAC1 gene.
55. The method according to embodiment 54, wherein said out-of-frame mutation inhibits proper TRAC1 protein generation or function.
56. The method according to any one of embodiments 41 or 42, wherein the guide RNA of said chimeric nuclease is SEQ ID NO:20, or a fragment thereof; wherein said chimeric nuclease targets TRCB1 gene.
57. The method according to embodiments 56, wherein said chimeric nuclease generates an out-of-frame mutation of the TRCB1 gene.
58. The method according embodiment 57, wherein said out-of-frame mutation inhibits proper TRCB1 protein generation or function.
59. The method according to any one of embodiments 41 or 42, wherein the guide RNA of said chimeric nuclease is SEQ ID NO:21, or a fragment thereof; wherein said chimeric nuclease targets and cleaves the HLA-A gene.
60. The method according to embodiment 59, wherein said chimeric nuclease generates an out-of-frame mutation of the HLA-A gene.
61. The method according to anyone of embodiments 60, wherein said out-of-frame mutation inhibits proper HLA-A protein generation or function.
62. The method according to any one of embodiments 41 or 42, wherein the guide RNA of said chimeric nuclease is SEQ ID NO: 22, or a fragment thereof; wherein said chimeric nuclease targets the AAVS1 gene.
63. The method according to embodiment 62, wherein the AAVS1 gene is targeted in the presence of exogenous donor DNA.
64. The method according to embodiment 63, wherein the safe harbor site in the AAVS1 gene is targeted.
65. The method according to embodiment 63, wherein the chimeric nuclease targets two sites on the AAVS1 gene.
66. The method according to embodiment 65, wherein the exogenous donor DNA is integrated between the two targeted sites on the AAVS1 gene.
67. The method according to any one of embodiments 63-66, wherein the exogenous donor DNA contains DNA sequences that express a chimeric antigen receptor.
68. The method according to embodiment 67, wherein the integrated exogenous donor DNA is retained via non-homologous end joining.
69. The method according to embodiment 67, wherein a chimeric antigen receptor targeting CD19 of SEQ ID NO: 7 is inserted into the AAVS1 site.
70. The method according to embodiment 63, wherein a LoxP site is inserted into the AAVS1.
71. The method according to embodiment 70, wherein the LoxP site is of SEQ ID NO: 24.
72. A method to generate immune tolerant cells comprising the step of administering a mixture of two or more chimeric nucleases according to embodiment 6.
73. The method to generate immune tolerant cells comprising the step of administering a mixture of chimeric nucleases according to embodiment 72, wherein said chimeric nucleases target one or more genes selected from the group consisting of B2M, TRAC1, TRBC1, HLA-A and AAVS1.
74. The method to generate immune tolerant cells according to embodiment 73, wherein said mixture of chimeric nucleases contains multiple guide RNAs selected from the group consisting of SEQ ID NOs: 17 or 18, 19, 20, and 21, or fragments thereof.
75. The method to generate immune tolerant cells according to embodiment 73, wherein said mixture of chimeric nucleases containing multiple guide RNAs are administered simultaneously.
76. The method to generate immune tolerant cells according to embodiment 75, wherein said mixture contains a multiple of guide RNAs to target multiple sequences within the same exogenous DNA of the target cell.
77. The method to generate immune tolerant cells according to embodiment 75, wherein the ratio of chimeric nucleases and guide RNAs is equimolar.
78. A formulation containing a chimeric nuclease according to embodiment 2, comprising a mixture of chimeric nucleases containing multiple guide RNAs selected from SEQ ID NOs: 17 or 18, 19, 20, and 21, or fragments thereof wherein said chimeric nucleases target multiple sequences within the same exogenous DNA of the target cell.
79. The formulation according to embodiment 76, wherein the ratio of chimeric nucleases and guide RNAs is equimolar.
80. A polypeptide according to embodiment 1, comprising the entire amino acid sequence of any one of SEQ ID NOs: 29-33 or fragments thereof.
81. A polypeptide according to embodiment 1, comprising the entire amino acid sequence of any one of SEQ ID NOs: 34-38 or fragments thereof.
82. A polypeptide according to embodiment 1, comprising the entire amino acid sequence of any one of SEQ ID NOs: 39-43 or fragments thereof.
83. A polypeptide according to embodiment 1, comprising the entire amino acid sequence of any one of SEQ ID NOs: 44-48 or fragments thereof.
84. A polypeptide comprising at least 80% of the amino acid sequence of SEQ ID NO: 29.
85. A polypeptide comprising at least 85% of the amino acid sequence of SEQ ID NO: 29.
86. A polypeptide comprising at least 90% of the amino acid sequence of SEQ ID NO: 29.
87. A polypeptide comprising at least 95% of the amino acid sequence of SEQ ID NO: 29.
88. A polypeptide comprising at least 80% of the amino acid sequence of SEQ ID NO: 34.
89. A polypeptide comprising at least 85% of the amino acid sequence of SEQ ID NO: 34.
90. A polypeptide comprising at least 90% of the amino acid sequence of SEQ ID NO: 34.
91. A polypeptide comprising at least 95% of the amino acid sequence of SEQ ID NO: 34.
92. A polypeptide comprising at least 80% of the amino acid sequence of SEQ ID NO: 39.
93. A polypeptide comprising at least 85% of the amino acid sequence of SEQ ID NO: 39.
94. A polypeptide comprising at least 90% of the amino acid sequence of SEQ ID NO: 39.
95. A polypeptide comprising at least 95% of the amino acid sequence of SEQ ID NO: 39.
96. A polypeptide comprising at least 80% of the amino acid sequence of SEQ ID NO: 44.
97. A polypeptide comprising at least 85% of the amino acid sequence of SEQ ID NO: 44.
98. A polypeptide comprising at least 90% of the amino acid sequence of SEQ ID NO: 44.
99. A polypeptide comprising at least 95% of the amino acid sequence of SEQ ID NO: 44.
100. A pharmaceutically-acceptable formulation comprising the chimeric nuclease according to embodiment 2.
101. A pharmaceutically-acceptable formulation comprising the chimeric nuclease according to embodiment 6.
102. The pharmaceutically-acceptable formulation according to embodiment 6, wherein said pharmaceutically-acceptable formulation comprises cells that underwent electroporation with a nuclease.
103. The pharmaceutically-acceptable formulation according to embodiment 100, wherein said chimeric nuclease is any one of SEQ ID NOs: 25-49.
104. The pharmaceutically-acceptable formulation according to embodiment 100, further comprising exogenous donor DNA.
105. A method to treat a disease comprising the step of administering the pharmaceutically-acceptable formulation according to embodiment 102.
106. The method according to embodiment 103, wherein said disease is cancer.
107. The method to treat a disease, comprising the step of generating immune cells according to embodiment 72, wherein said chimeric nuclease modifies the DNA of immune cells.
108. The method to treat a disease according to embodiment 105, wherein said disease is cancer.
109. A method support immune tolerance in a human comprising the step of administering the pharmaceutically-acceptable formulation according to embodiment 102.
110. The method support immune tolerance according to embodiment 109, comprising the step of generating immune cells according to embodiment 72, wherein said chimeric nuclease modifies the DNA of immune cells.
111. The method according to embodiment 33, wherein said exogenous donor DNA is selected from the group consisting of double-stranded DNA, double-stranded DNA with non-complimentary ends or circular double-stranded DNA.
112. The method according to embodiment 111, wherein the polynucleotide chains of said double-stranded DNA are of the same length and were both cleaved by the same chimeric nuclease according to embodiment 6.
113. The method according to embodiment 111, wherein said non-complimentary DNA ends were cleaved by the same chimeric nuclease according to embodiment 6.
114. The method according to embodiment 111, wherein said double-stranded DNA or said double-stranded DNA with non-complimentary ends are both phosphorylated at the 5′ end or chemically-modified.
115. The method according to embodiment 111, wherein said double-stranded DNA or said double-stranded DNA with non-complimentary ends contain one or more phosphorothioate bonds.
116. The method according to embodiment 111, wherein said double-stranded DNA or said double-stranded DNA with non-complimentary ends are chemically-modified with biotin.
117. The method according to embodiment 111, wherein said circular double-stranded DNA comprises I-TevI and SaCas9 target sites.
118. The chimeric nuclease according to embodiment 6, wherein said Staphylococcus aureus Cas9 domain is one of SEQ ID NOs: 25-28.
119. The chimeric nuclease according to embodiment 6, wherein the RNA-guided nuclease Staphylococcus aureus Cas9 domain contains sequences to account for genetic polymorphisms.
120. The chimeric nuclease according to embodiment 6, wherein the RNA-guided nuclease Staphylococcus aureus Cas9 domain targets safe harbor sites on the genomic DNA in the target cells.
121. The chimeric nuclease according to embodiment 120, wherein the safe harbors are hROSA26 and CCR5.
122. The method according to embodiment 7, wherein said chimeric nuclease is delivered using lipid nanoparticles, polymers, viral vectors or cell-penetrating peptides.
123. A method according to embodiment 20, wherein the cell is a lung cell.
124. A method according to embodiment 20, wherein the chimeric nuclease is the polypeptide of SEQ ID NO: 31.
125. A method according to embodiment 20, wherein the DNA expression cassette is delivered to treat a genetic disease.
126. A chimeric nuclease with two active nuclease sites comprising a modified I-TevI nuclease domain, a linker and a second nuclease domain, wherein the nuclease activity of the active sites is coordinated.
127. A chimeric nuclease of embodiment 126, wherein the second nuclease is a modified RNA-guided nuclease and further comprises a guide RNA.
128. A method to edit genomic DNA comprising the step of administering the chimeric nuclease according to embodiment 126 to a cell or organism.
129. A method to delete defined lengths of a DNA molecule comprising the step of delivering the chimeric nuclease according to embodiment 128 to a cell or organism.
130. A method to replace select sequences from a DNA molecule comprising the step of delivering the chimeric nuclease according to embodiment 128 to a cell or organism.
131. A polypeptide comprising at least 80% of the amino acid sequence of SEQ ID NO: 49.
132. A polypeptide comprising at least 85% of the amino acid sequence of SEQ ID NO: 49.
133. A polypeptide comprising at least 90% of the amino acid sequence of SEQ ID NO: 49.
134. A polypeptide comprising at least 95% of the amino acid sequence of SEQ ID NO: 49.

### Definitions and Acronyms

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

Within the framework of the present description and in the subsequent claims, except where otherwise indicated, all numbers expressing amounts, quantities, percentages, and so forth, are to be understood as being preceded in all instances by the term “about”. As used herein, the term “about” is defined as ±10%. Also, all ranges of numerical entities include all the possible combinations of the maximum and minimum numerical values and all the possible intermediate ranges therein, in addition to those specifically indicated hereafter.

The term “and/or” as used herein is defined as the possibility of having one or the other or both. For example, “A and/or B” provides for the scenarios of having just A or just B or a combination of A and B. If the claim reads A and/or B and/or C, the composition may include A alone, B alone, C alone, A and B but not C, B and C but not A, A and C but not B or all three A, B and C as components.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity, as computed using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Any of the polypeptides, I-TevI domains, linker domains, or Cas polypeptide nucleases described herein can have at least about 80%, 85%, 90%, 95%, 96%, 97%, 98% or 99% identity to or be identical to any of the listed polypeptides described in the sequence listing provided herewith. In certain embodiments, a polypeptide comprising the recited sequence identity can preserve the nuclease activity of a wild-type version of the polypeptide or of the altered nuclease activity conferred by any one of the mutations described herein (e.g., D10E mutation of Cas9; or a (K26R, a T95S, and a Q158R mutation), (V117F mutation) (K135R/N140S), (V117F/K135R/N140S) of I-TevI).

In some embodiments, amino acid sequence variants of the polypeptides and chimeric nucleases provided herein are contemplated. A variant typically differs from a polypeptide specifically disclosed herein in one or more substitutions, deletions, additions and/or insertions. Such variants can be naturally occurring or can be synthetically generated, for example, by modifying one or more of the polypeptide sequences described herein and evaluating one or more biological activities of the polypeptide as described herein and/or using any of a number of known techniques. Any combination of deletion, insertion, and substitution can be made to arrive at the final construct, provided that the final construct possesses the desired characteristics, e.g., antigen-binding.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application. Any of the nucleic acids or guide RNAs described herein can comprise at least about 80%, 85%, 90%, 95%, 97%, 98%, 99% or 100% homology to the recited sequence identifier, while maintaining the appropriate binding activities (e.g., to gene or nuclease) conferred by the recited sequence.

The articles “a” and “an” are used herein to refer to one or to more than one (i.e., to at least one) of the grammatical object of the article. The term “and/or” as used herein is defined as the possibility of having one or the other or both. For example, “A and/or B” provides for the scenarios of having just A or just B or a combination of A and B. If the claim reads A and/or B and/or C, the composition may include A alone, B alone, C alone, A and B but not C, B and C but not A, A and C but not B or all three A, B and C as components.

The terms “administer” and “administering” are used herein to refer to the dispensation or application of a drug, medication, or other form of a therapeutic agent to a patient suffering from a disease or condition.

A “buffer” as used herein is any acid or salt combination which is pharmaceutically acceptable and capable of maintaining the composition of the present invention within a desired pH range. Buffers in the disclosed compositions maintain the pH in a range of about 2 to about 8.5, about 5.0 to about 8.0, about 6.0 to about 7.5, about 6.5 to about 7.5, or about 6.5. Suitable buffers include any pharmaceutical acceptable buffer capable of maintaining the above pH ranges, such as, for example, acetate, tartrate phosphate or citrate buffers. In one embodiment, the buffer is a phosphate buffer. In another embodiment the buffer is an acetate buffer. In one embodiment the buffer is disodium hydrogen phosphate, sodium chloride, potassium chloride and potassium phosphate monobasic.

The term “chimeric antigen receptor”, as used herein, refer to T cells that have been genetically engineered to produce an artificial T-cell receptor for use in immunotherapy (also known as CAR T cells).

The term “chimeric nuclease”, as used herein, refers to engineered proteins which comprise one or more DNA-binding domains to give sequence specificity and one or more nuclease domains for DNA cleavage.

The terms “cleave” or “to cleave” or “cleaved” or “cleavage”, as used herein, refer to splitting of one or more phosphodiester bonds of a DNA molecule. Cleavage of double-stranded DNA at a single target site results in two DNA fragments, which may be repaired by non-homologous end-joining or homology-directed repair. Cleavage of double-stranded DNA at two target sites results in three fragments, which may be repaired by non-homologous end-joining or homology-directed repair with loss or deletion of the middle fragment. With respect to the instant application, “cleavage” of genomic DNA at two targets sites can create predictable length deletions.

The term “defined”, as used herein in reference to DNA or RNA fragments or target sites, refers to having a specific nucleotide length or a specific nucleotide sequence.

The terms “deliver” or “to deliver” or “delivered” or “delivery”, as used herein, refer to sending gene-editing tools (e.g., a nuclease, gRNA and/or exogenous donor DNA) directly to target cells to effect a genetic modification of the genomic DNA of the cell in vivo or ex vivo.

The term “genetic engineering” and grammatical equivalents refers to the introduction of one or more deletions, insertions, or substitutions into the genome of a target cell or individual.

The terms “disease” or “diseased”, as used herein, refer to a disorder of structure or function in a human, animal, or plant, especially one that produces specific signs or symptoms or that affects a specific location and is not simply a direct result of physical injury.

The term “disrupt”, as used herein, refers to any process by which expression or function of a gene is inhibited or inactivated. To “disrupt expression” refers to altering, inhibiting or eliminating the process by which genetic information is used in the synthesis of a functional gene product, often proteins.

The terms “domain” and “domains”, as used herein, refer to a conserved part of a given protein sequence and tertiary structure that can evolve, function, and exist independently of the rest of the protein chain. Each domain forms a compact three-dimensional structure and often can be independently stable and folded. Many proteins consist of several structural domains. One domain may appear in a variety of different proteins. Molecular evolution uses domains as building blocks and these may be recombined in different arrangements to create proteins with different functions. In general, domains vary in length from between about 50 amino acids up to 1,500 amino acids in length. Domains often form functional units, such as the calcium-binding EF hand domain of calmodulin. Because they are independently stable, domains can be “swapped” by genetic engineering between one protein and another to make chimeric proteins.

The terms “edit” or “to edit” or “editing”, as used herein, refer to a group of technologies to change an organism's genomic DNA. These technologies allow genetic material to be added, removed, or altered at particular locations in the genome.

As used herein, an “effective amount” refers to an amount sufficient to elicit the desired response. In the present invention, the desired biological response is the genetic modification of cells, including T-cells and pluripotent stem cells.

The term “electroporation”, as used herein, refers to a microbiology technique in which an electrical field is applied to cells in order to increase the permeability of the cell membrane, allowing chemicals, drugs, DNA, or proteins to be introduced into the cell.

The term “endogenous”, as used herein, refers to growing or originating from within an organism.

The term “equimolar”, as used herein, refers to of or relating to an equal number of moles.

The term “ex vivo”, as used herein, refers to experimentation or measurements done in or on tissue from an organism in an external environment with minimal alteration of natural conditions. Ex vivo means that the samples to be tested have been extracted from the organism. Ex vivo experimentation enables usage of organism's cells or tissues under more controlled conditions than is possible in in vivo experiments (in the intact organism). Examples of ex vivo specimen use include: (1) assays; (2) finding cancer treatment agents that are effective against the organism's cancer cells; (3) measurements of physical, thermal, electrical, mechanical, optical and other tissue properties, especially in various environments that may not be life-sustaining (for example, at extreme pressures or temperatures); (4) realistic models for surgical procedure development; (5) investigations into the interaction of different energy types with tissues. In the present invention, ex vivo includes the editing of cells or tissue with the purpose of transplanting or administering the edited cells or tissue to a subject in need thereof.

The term “donor DNA” or “exogenous donor DNA” as used herein refers to DNA that is supplied to cell in conjunction with a nuclease described herein that is intended to be inserted into the genome by any method such as non-homologous end joining, homology directed repair, microhomology directed repair, or any combination of the above. Donor DNA may insert a nucleotide sequence comprising a genetic element such as an open reading frame, an exon, an intron, or an expression cassette that is not naturally present in the organism or cell; or may repair or replace an existing gene. Alternatively, donor DNA may be inserted to disrupt an endogenous gene by introducing a missense, frame-shift mutation or stop codon mutation into a codon region or regulatory element (e.g., promoter, splice donor/acceptor). Donor DNA can be supplied to a cell or an organism described herein by way of non-limiting example, by a plasmid, a linear single- or double-stranded DNA, a synthetic single- or double-stranded DNA, or a viral vector. Donor DNA is distinguished from nucleic acids and/or delivery vectors that encode the chimeric nucleases and guide RNAs described herein.

The term “expression cassette”, as used herein, refers to a distinct component of vector DNA consisting of a gene operably linked to a regulatory sequence to be expressed by a transfected cell. In each successful transfection, the expression cassette directs the cell's machinery to make RNA(s) and protein(s). Some expression cassettes are designed for modular cloning of protein-encoding sequences so that the same cassette can easily be altered to make different proteins. An expression cassette is composed of one or more genes and the sequences controlling their expression. An expression cassette comprises three components: a promoter sequence, an open reading frame, and a 3′ untranslated region that, in eukaryotes, usually contains a polyadenylation site. Different expression cassettes can be transfected into different organisms including bacteria, yeast, plants, and mammalian cells as long as the correct regulatory sequences are used.

The terms “fragment” or “fragments” or “fragments thereof”, as used herein, refer to a small part broken from a larger entity. Fragments described herein when referring to nucleases such as Cas nucleases or I-TevI nucleases comprise amino acid residue deletions from the N-terminus, C-terminus, or a defined internal deletion that preserve the enzymatic activity of the nuclease. Deletions can comprise 1, 2, 3, 4, 5, 6, 7, 8, 9, 10 or more amino acid deletions from N-terminus, C, terminus, or an internal sequence of the polypeptides described herein.

The term “functional gene”, as used herein, refers to a portion of DNA coding for one polypeptide chain or other gene product. The ‘one gene/one enzyme’ hypothesis thus becomes the ‘one cistron (gene)/one polypeptide’ hypothesis or ‘one gene/one functional product’ hypothesis.

The terms “genomic DNA” or “gDNA”, as used herein, refer to the chromosomal DNA of an organism, representing the bulk of its genetic material. It is distinct from bacterial plasmid DNA, complementary DNA, or mitochondrial DNA.

The term “immune-tolerant” refers to a state of unresponsiveness of the immune system to substances or tissue that have the capacity to elicit an immune response in a given organism.

The term “including”, as used herein, is used to mean “including but not limited to”. “Including” and “including but not limited to” are used interchangeably.

The term “independent sites”, as used herein, refers to areas on or in a target, such as a DNA molecule or a protein/peptide, that are connected with another or with each other but are not the same sequence and are considered separate.

The term “induced pluripotent stem cells”, as used herein, refers to a type of pluripotent stem cell that can be generated directly from a somatic cell.

The terms “inhibit” or “to inhibit” or “inhibits” or “inhibiting” or “inhibition” or “inhibition thereof”, as used herein and particular to the claimed invention, refer to the ability to slow down or prevent a function or reduce the activity of an enzyme or other agent.

The term “insert” as used herein, is used to mean the addition of an entire gene or defined length of DNA molecule to cells, or the addition and/or substitution, of a subset of nucleotides to a gene.

The term “linker”, as used herein, refers to a situation when the RNA-guided nuclease domain (Cas9) binds to the target DNA sequence, the amino acid linker domain ensures mobility of the I-TevI domain to allow for recognition, binding and cleaving of its target sequence under cell physiological conditions (typically: pH 7.2, temperature≈37° C., [K+]≈140 mM, [Na+]≈5-15 mM, [Cl−]≈4 mM, [Ca++]≈0.0001 mM). The length of the amino acid linker can influence how many nucleotides are preferred between the Cas9 target site and the I-TevI target site. Certain amino acids in the linker may also make specific contacts with the DNA sequence targeted by TevCas9. These linker-DNA contacts can affect the flexibility of the I-TevI domain. Substituting amino acids in the linker domain may affect the ability of the linker domain to make contact with DNA.

The terms “modify” or “to modify” or “modifies” or “modified” or “modification”, as used herein, refer to a technique to change the characteristics of a plant, animal or micro-organism through the alterations of nucleotides from desired genes within the DNA of an organism. Alterations can include the removal and/or addition of nucleotides including entire genes, and/or fragments thereof. The terms refer to making basic or fundamental changes in order to give new function, such as changing the DNA of immune cells (such as T-cells) or induced pluripotent stem cells (iPSCs) by engineering said cells for immune tolerance and/or to express exogenous genes, such as a chimeric antigen receptor (CAR), although not limited to just those cell types. With respect to the instant application, it generally refers to the addition or deletion of nucleotides in the DNA in a cell. In particular, it refers to a synthesized version of an I-TevI domain, a linker peptide and a modified version of an RNA-guided nuclease designed to target genes to encourage immunogenicity in known cell therapies.

The term “non-homologous end joining”, as used herein, refers to a pathway that repairs double-strand breaks in DNA. NHEJ is referred to as “non-homologous” because the break ends are directly ligated without the need for a homologous template, in contrast to homology directed repair, which requires a homologous sequence to guide repair.

The term “nuclease”, as used herein, refers to an enzyme that cleaves the phosphodiester bonds between nucleotides of nucleic acids.

The term “nuclease domain”, as used herein, refers to an area on a target that is responsible for physical cleavage of DNA strands and may introduce either single stranded or double-stranded breaks. For example, in some embodiments, a nuclease domain can refers to amino acids 1-92 of the wild-type I-TevI nuclease domain or to variants thereof with altered amino acid sequences and/or binding or nuclease properties.

The term “out-of-frame mutation” or “frame shift mutation”, as used herein, refers to the removal or addition of one or more nucleotides which severely disrupts the production of the protein resulting in a completely non-functional protein or not producing a protein at all.

The term “patient,” “subject” or “host” to be treated by the subject method may mean either a human or non-human animal. Non-human animals include companion animals (e.g. cats, dogs) and animals raised for consumption (i.e. food animals), such as cows, pigs, and chickens.

The term “pharmaceutically acceptable formulation” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as dextrose, lactose, glucose and sucrose; (2) starches, such as corn starch and potato starch; (3) cellulose, and its derivatives, such as microcrystalline cellulose, sodium carboxymethyl cellulose, methyl cellulose, ethyl cellulose, hydroxypropylmethyl cellulose (HPMC), and cellulose acetate; (4) glycols, such as propylene glycol; (5) polyols, such as glycerin, sorbitol, mannitol and polyethylene glycol; (6) esters, such as ethyl oleate, glyceryl behenate and ethyl laurate; (7) buffering agents, such as monobasic and dibasic phosphates, Tris/Borate/EDTA and Tris/Acetate/EDTA (8) pyrogen-free water; (9) isotonic saline; (10) Ringer's solution; (11) ethyl alcohol; (12) phosphate buffer solutions; (13) polysorbates; (14) polyphosphates; and (15) other non-toxic compatible substances employed in pharmaceutical formulations. The disclosed excipients may serve more than one function. For example, a solubilizing agent may also be a suspension aid, an emulsifier, a preservative, and the like.

The term “pluripotent stem cells”, as used herein, refers to types of cells that have the ability to undergo self-renewal and give rise to any cell of the tissue of the body.

A T cell as described herein is a type of immune cell involved in adaptive cellular immune responses. T cells are identified by expression of the cell surface molecule CD3. T cells may be utilized as therapeutic cells when they express a chimeric antigen receptor (CAR).

An NK cell as described herein is a type of immune cell involved in immune responses. NK cells are identified by expression of the cell surface molecule CD56. NK cells may be utilized as therapeutic cells when they express a chimeric antigen receptor (CAR).

A hematopoietic stem cell (HSCs) as described herein is an immature cell that can develop into all types of blood cells, including white blood cells, red blood cells, and platelets. Hematopoietic stem cells are found in the peripheral blood and the bone marrow. Also called blood stem cell. Hematopoietic stem cells are identified by expression of the cell surface molecule CD34.

A mesenchymal stem cell as described herein are multipotent stromal cells that can differentiate into a variety of cell types, including osteoblasts (bone cells), chondrocytes (cartilage cells), myocytes (muscle cells) and adipocytes (fat cells).

The term “polypeptide”, as used herein, refers to a linear organic polymer consisting of a large number of amino-acid residues bonded together in a chain, forming part of (or the whole of) a protein molecule.

The term “proper”, as used herein in reference to protein generation or function, refers refers to generation of a substantially normal or native form of a protein that substantially retains its biological effect or activity or is suitable for its intended uses as described herein.

The terms “RNA-guided” or “guide RNA”, as used herein, refer to prokaryotic DNA editing involving CRISPR and Cas9. For this prokaryotic DNA-editing system, the RNA-guided nuclease confers target sequence specificity to the CRISPR-Cas9 system. These gRNAs are non-coding short RNA sequences which bind to the complementary target DNA sequences.

The term “select DNA”, as used herein, refers to being chosen or isolated from a larger number of DNA sequences.

The term “suitable”, as used herein, refers to sufficiently adapted to a use or purpose.

The terms “target site” or “target sites”, as used herein, refer to, with respect to the present invention in particular, a position in a gene that when targeted with a nuclease will be bound and/or cleaved by the nuclease. A target site can encompass a multiplicity of nucleotides, such as the cleavage site of an I-TevI or CRISPR/Cas nuclease, or the DNA binding site of an I-TevI nuclease or CRISPR/Cas guide RNA. A target site can be in a gene or intergenic region in any of various cell types and organisms.

The terms “target” or “targets” or “to target” or “targeting”, as used herein, refer to, with respect to the inventions of the instant application, aiming or directing a nuclease to a particular, selected DNA sequence using, for example, a selected or engineered DNA binding domain or guide RNA.

The term “viral vector”, as used herein, refers to tools commonly used to deliver genetic material into cells. This process can be performed inside a living organism (in vivo) or in cell culture (in vitro). Examples of viral vectors include AAV vectors, lentiviral vectors, and adenoviral vectors.

The term “simultaneously”, as used herein, refers to the administration of CRISPR/Cas9 complex with multiple gRNA at the same time or substantially the same time. It will be understood that certain procedures can be executed in steps that occur sequentially but are spaced apart by small intervals. For example, electroporation of cells followed by administration of a donor DNA within about an hour.

The term “substitution”, as used herein, refers to the replacement of an amino acid in a sequence with a different amino acid. As used herein, the shorthand X10Y indicates that amino acid Y has been “substituted” for amino acid X found in the 10th position of the sequence. As an example, W26C denotes that amino acid Tryptophan-26 (Trp, W) is changed to a Cysteine (Cys). Similarly, the notation AAX indicates that AA is an amino acid that replaced the amino acid found in the X position. As an example, Lys26 denotes the replacement of the amino acid in the 26th position in a sequence with Lysine. Use of either shorthand is interchangeable. In addition, use of the one- or three-letter abbreviations for an amino acid is also interchangeable.

The term “treating”, as used herein, includes any effect, e.g., lessening, reducing, modulating, or eliminating, that results in the improvement of the condition, disease, disorder, and the like. As used herein, “treating” can include both prophylactic, and therapeutic treatment. For example, therapeutic treatment can include delaying inhibiting or preventing the progression of cystic fibrosis or non-small cell lung cancer, the reduction or elimination of symptoms associated with cystic fibrosis or non-small cell lung cancer. Prophylactic treatment can include preventing, inhibiting or delaying the onset of cystic fibrosis or non-small cell lung cancer.

### Abbreviations

Abbreviations used herein are defined as follows:
AA amino acid
AAVS1 adeno-associated virus integration site 1
B2M Beta-2 microglobulin
CAR Chimeric antigen receptor
Cas9 CRISPR-associated protein 9
Cpf1 CRISPR from Prevotella and Francisella 1
CRISPR Clustered Regulatory Interspaced Short Palindromic Repeats
CFTR Cystic fibrosis transmembrane conductance regulator gene
DNA deoxyribonucleic acid
E. Coli Escherichia coli
EDTA ethylenediaminetetraacetic acid
EGFR Epidermal growth factor receptor
HDR Homology directed repair
HLA-A histocompatibility antigen, A alpha chain
iPSCs induced pluripotent stem cells
NHEJ Non-homologous end joining
NLS Nuclear-localized signal
PCR polymerase chain reaction
RNA ribonucleic acid
saCas9 Staphylococcus aureus Cas9
scCas9 Streptococcus canis Cas9
spCas9 Streptococcus pyogenes Cas9
TALEN Transcription activator-like effector nucleases
TCR T cell receptor
TevCas9 Modified I-TevI domain, a linker peptide and modified RNA-guided nuclease Staphylococcus aureus Cas9
TRAC1 T cell receptor alpha chain constant
TRBC1 T cell receptor beta constant 1
ZFN zinc-finger nucleases

### EXAMPLES

The following illustrative examples are representative of embodiments of compositions and methods described herein and are not meant to be limiting in any way.

### Example 1—TevSaCas9 Cleaves Target Sites

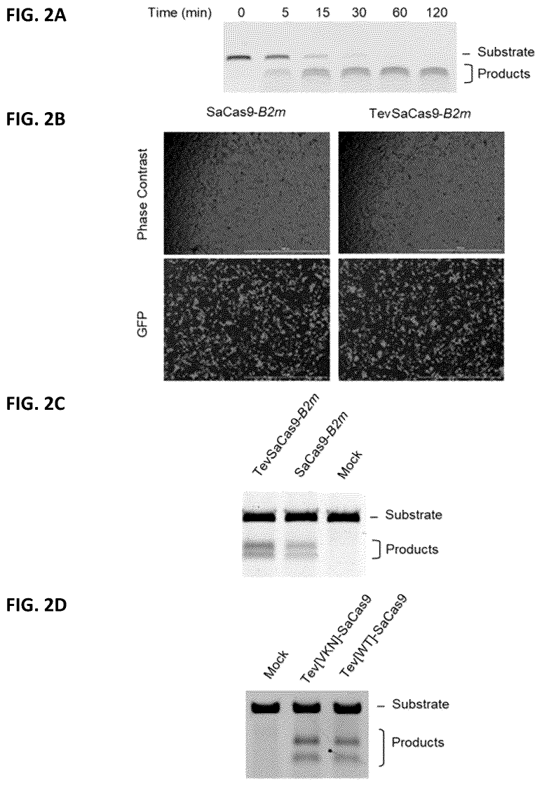

FIG. 2A illustrates that TevSaCas9 targeted to the B2M gene using guide RNA in SEQ ID 17 cleaves B2M DNA substrate in vitro. The products of the cleavage reaction over time are visualized on an agarose gel. FIG. 2B are mammalian cells transfected with a plasmid DNA version of TevSaCas9 fused to a cleavable GFP tag are imaged using phase contrast and GFP imaging on a Cytation5 (Biotek Instruments Inc, VT, USA) after 48 hours treatment. FIG. 2C evidences editing at the B2M gene by plasmid DNA encoded TevSaCas9 as detected by PCR amplification and T7 Endonuclease I cleavage assay of genomic DNA extracted from harvested cells. FIG. 2D evidences editing at the B2M gene using purified TevSaCas9 protein of SEQ ID 29 or TevSaCas9 protein of SEQ ID 32 containing the V117F/K135R/N140S mutations complexed with guide RNA targeting B2M of SEQ ID 17 and transfected into mammalian cells. Genomic DNA is extracted from harvested cells and editing at the B2M gene is detected by PCR amplification and a T7 Endonuclease I cleavage assay.

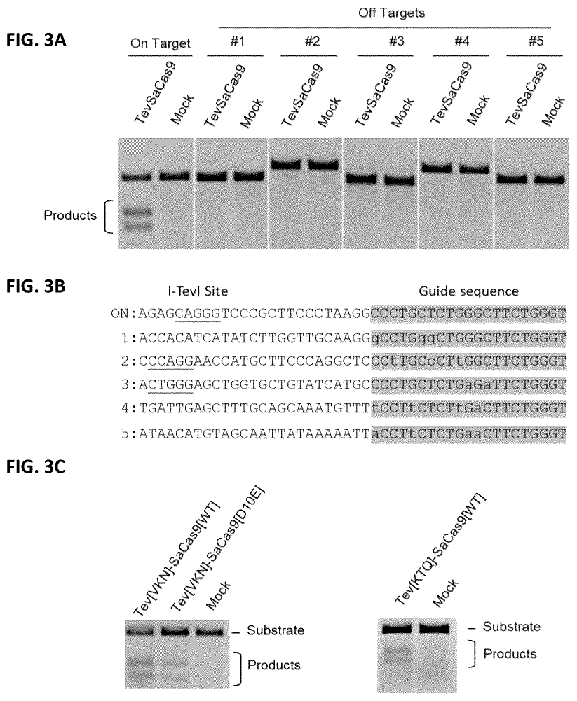

FIG. 3A illustrates that TevSaCas9 targeted to the AAVS1 gene using guide in SEQ ID 22 cleaves the AAVS1 target site in the genome of mammalian cells (on target) but not off-target sequences. Genomic DNA is extracted from harvested cells and editing at the AAVS1 gene is detected by PCR amplification and a T7 Endonuclease I cleavage assay. Editing at the top 5 off target sites predicted in silico were also measured by T7 Endonuclease I cleavage assay with no off targets detected. FIG. 3B shows the sequences of on target and off target sites for AAVS1. Grey highlighted sequence indicated guide RNA target site. Lowercase nucleotides indicate mismatch positions to the on target. Underlined sequences indicate a putative I-TevI binding and cleavage site. FIG. 3C evidences the activity of Tev[VKN]-SaCas9[D10E] of SEQ ID 37 and Tev[KTQ]-SaCas9[WT] of SEQ ID 33 at the AAVS1 gene in mammalian cells. Genomic DNA is extracted from harvested cells and editing at the AAVS1 gene is detected by PCR amplification and a T7 Endonuclease I cleavage assay.

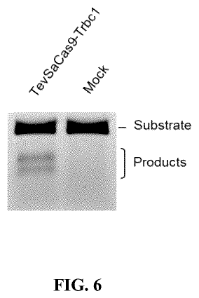

FIG. 6 illustrates that TevSaCas9 targeted to the TRBC1 gene using guide in SEQ ID 20 cleaves the TRBC1 target site in the genome of mammalian cells. Genomic DNA is extracted from harvested cells and editing at the TRBC1 gene is detected by PCR amplification and a T7 Endonuclease I cleavage assay.

### Example 2—TevSaCas9 Inserts Donor DNA into a Specific Genomic Site

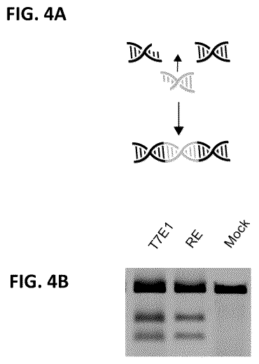

FIG. 4A shows a diagram representing the target site in the B2M gene after TevSaCas9 cleavage (black) and insertion of a donor oligonucleotide repair template (grey) into the cut site and that oligonucleotide repair occurs. FIG. 4B illustrates the insertion of the donor oligonucleotide repair template. The results of the T7 Endonuclease I cleavage assay (“T7E1”) and restriction enzyme digestion (“RE”) of cells transfected with TevSaCas9 to the B2M and repair template harbouring a unique restriction enzyme site as shown. Genomic DNA is extracted from harvested cells and editing at the B2M gene is detected by PCR amplification and a T7 Endonuclease I cleavage assay. Insertion of the oligonucleotide repair template is measured by restriction enzyme digestion.

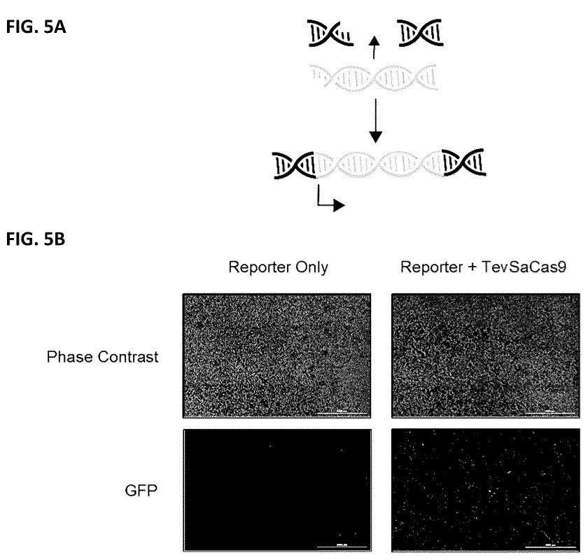

FIGS. 5A and 5B is a diagram depicting the AAVS1 safe harbour site within the genome (black) after cleavage by TevSaCas9. Shown in grey is an approximately 1.2 kilobase green fluorescent protein (GFP) reporter construct with ends that are compatible with the TevSaCas9 cut site. GFP fluorescence was observed in reporter plus TevSaCas9 cells, but not in cells with reporter only. FIG. 5B evidences insertion and expression of the GFP reporter in the AAVS1 site in HEK293 cells. Cells treated with TevSaCas9 with and without the reporter construct were imaged 48 hours after co-transfection. The presence of GFP positive cells indicate expression of the GFP reporter.

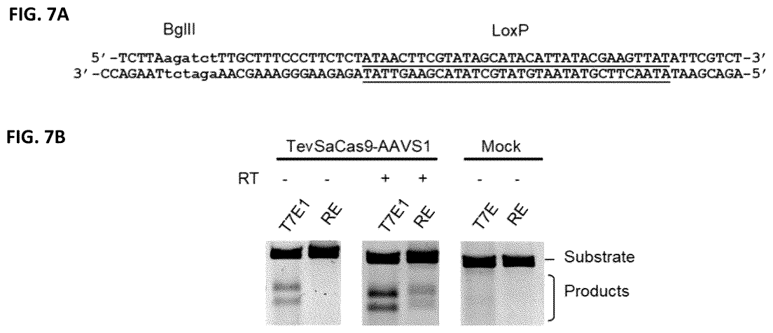

FIG. 7A is a diagram of the sequence of the double strand DNA oligonucleotide repair template containing a single LoxP site (“RT”). Shown in lowercase is the BglII restriction enzyme site and underlined is a single LoxP site. FIG. 7B evidences the insertion of the double strand DNA oligonucleotide containing a single LoxP site into the AAVS1 target site. Shown are the results of the T7 Endonuclease I cleavage assay (“T7E1”) and restriction enzyme digestion (“RE”) of cells transfected with TevSaCas9 target to AAVS1 with and without repair template (RT) harbouring a BglII and LoxP site. Genomic DNA is extracted from harvested cells and editing at the AAVS1 gene is detected by PCR amplification and the T7E1 assay. Insertion of the oligonucleotide repair template is measured by RE digestion. Cells treated with TevSaCas9 which included the RT show RE digestion products of the expected size indicating insertion of the RT at the AAVS1 target site.

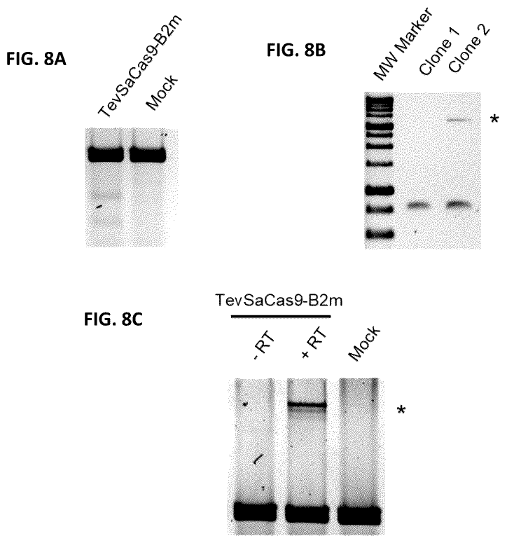

FIG. 8A illustrates that electroporated ribonucleoprotein TevSaCas9 edits the B2M target site in mammalian cells. HEK293 cells were electroporated with 1.5 μM of TevSaCas9 ribonucleoprotein complex targeted to B2M using 1150 volts with 2 pulses of 20 milliseconds each on a Neon® Transfection System (Thermo Fisher Scientific®, Waltham, Massachusetts, US). Results of the T7 Endonuclease I cleavage assay (“T7E1”) on TevSaCas9 treated or mock electroporated cells are shown. Genomic DNA is extracted from harvested cells and editing at the B2M gene is detected by PCR amplification and a T7 Endonuclease I cleavage assay. FIG. 8B evidences that donor DNA can be incorporated into the B2M target site using electroporated TevSaCas9 ribonucleoprotein complex. HEK293 cells were electroporated as in (A) except 2 μg of linearized donor DNA (RT) containing a green fluorescent protein (GFP) reporter was included. GFP-positive cells were then sorted to single cells using fluorescent-activated cell sorting and clonal populations of cells were expanded. The presence of the GFP reporter was detected in the clones by extracting a sample of genomic DNA and amplifying the B2M locus. A band on an agarose gel (denoted with *) corresponding to the size of the donor DNA construct is observed in Clone 2 indicating successful integration of the reporter into the B2M target site. FIG. 8C evidences that induced donor DNA can be incorporated into the B2M target site in pluripotent stem cells (iPSCs). iPSCs were electroporated with 7.5 μg of TevSaCas9 ribonucleoprotein complex targeted to B2M with and without 2 μg of linearized donor DNA (“RT”) containing an mCherry fluorescent reporter. A mock electroporation was conducted on with cells only. Genomic DNA was extracted from harvested cells and the B2M locus amplified by PCR. A band on an agarose gel (denoted with *) corresponding to the size of the donor DNA construct is observed only in the presence of the RT indicating insertion of the donor DNA into the B2M locus.

### Example 3—TevSaCas9 with Different Nuclear Localization Signals (NLSs) Cleave Target Sites

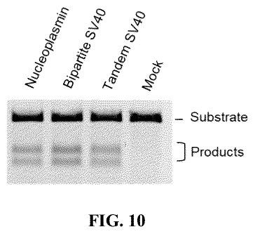

FIG. 10 illustrates cleavage of the B2M gene by TevSaCas9 of SEQ ID 32 in HEK293 cells with different nuclear localization signals (NLSs). Shown as the results of the T7 Endonuclease I cleavage assay (“T7E1”) of TevSaCas9 targeted to the B2M gene with two C-terminal nucleoplasmin NLSs of SEQ ID 56 separated by a human influenza hemagglutinin (HA) sequence (“Nucleoplasmin”), two SV40 NLSs of SEQ ID 55 separated by a HA sequence (“Bipartite SV40”) or a HA sequence followed by two SV40 NLSs (“Tandem SV40”). Also shown are cells treated with lipofection reagent (“Mock”). Genomic DNA is extracted from harvested cells and editing at the B2M gene is detected by PCR amplification and a T7 Endonuclease I cleavage assay.

### Example 4—Double Strand DNA Cleavage by a Fusion of I-TevI to Cas12a

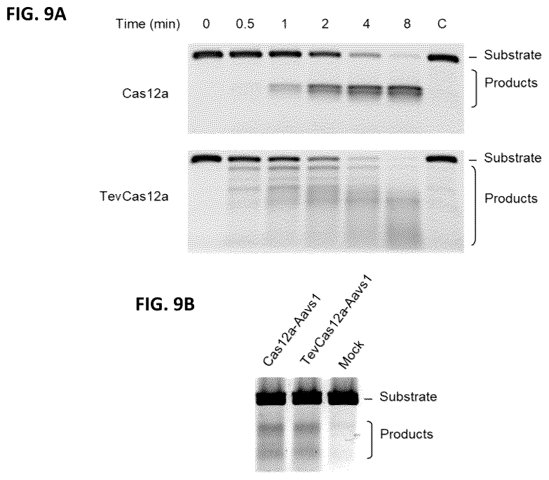

FIGS. 9A and 9B illustrates that a fusion of I-TevI to Cas12a of SEQ ID 47 is activated by guide RNA to cleave double-strand DNA substrate. Purified Cas12a and TevCas12 targeted to the AAVS1 gene using guide RNA in SEQ ID 50 cleaves AAVS1 DNA substrate in vitro. The lane marked “C” indicated protein only sample. The products of the cleavage reaction over time are visualized on an agarose gel. The two bands resulting from Cas12a cleavage indicate a single cut. Unexpectedly, several cleavage products are observed TevCas12a indicating that in the presence of guide RNA, TevCas12a cleaves double strand DNA non-specifically. FIG. 9B evidences that TevCas12a cleaves the AAVS1 target site in cells. HEK293 cells were lipofected with Cas12a and TevCas12a targeted to AAVS1. Genomic DNA was extracted from harvested cells and editing at the AAVS1 gene was detected by PCR amplification and a T7 Endonuclease I cleavage assay. The presence of significantly more products in the Cas12a and TevCas12a samples indicate TevCas12a cleaves the AAVS1 gene in cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

The above discussion is meant to be illustrative of the principle and various embodiments of the present invention. Numerous variations, combinations and modifications will become apparent to those skilled in the art once the above disclosure is fully appreciated. It is intended that the following claims be interpreted to embrace all such variations and modifications.

Sequences

SEQ ID NO 1
ATTCCTGAAGCTGACAGCATTCGGGCCGAGATGTCTCGCTCCGTGGCCTTAGCTGTG
CTCGCGCTACTCTCTCTTTCTGGCCTGGAGGCTATCCAGCGTACTCCAAAGATTCAG
GTTTACTCACGTCATCCAGCAGAGAATGGAAAGTCAAATTTCCTGAATTGCTATGTG
TCTGGGTTTCATCCATCCGACATTGAAGTTGACTTACTGAAGAATGGAGAGAGAATT
GAAAAAGTGGAGCATTCAGACTTGTCTTTCAGCAAGGACTGGTCTTTCTATCTCTTG
TACTACACTGAATTCACCCCCACTGAAAAAGATGAGTATGCCTGCCGTGTGAACCAT
GTGACTTTGTCACAGCCCAAGATAGTTAAGTGGGATCGAGACATGTAAGCAGCATC
ATGGAGGTTTGAAGATGCCGCATTTGGATTGGATGAATTCCAAATTCTGCTTGCTTG
CTTTTTAATATTGATATGCTTATACACTTACACTTTATGCACAAAATGTAGGGTTATA
ATAATGTTAACATGGACATGATCTTCTTTATAATTCTACTTTGAGTGCTGTCTCCATG
TTTGATGTATCTGAGCAGGTTGCTCCACAGGTAGCTCTAGGAGGGCTGGCAACTTAG
AGGTGGGGAGCAGAGAATTCTCTTATCCAACATCAACATCTTGGTCAGATTTGAACT
CTTCAATCTCTTGCACTCAAAGCTTGTTAAGATAGTTAAGCGTGCATAAGTTAACTTC
CAATTTACATACTCTGCTTAGAATTTGGGGGAAAATTTAGAAATATAATTGACAGGA
TTATTGGAAATTTGTTATAATGAATGAAACATTTTGTCATATAAGATTCATATTTACT
TCTTATACATTTGATAAAGTAAGGCATGGTTGTGGTTAATCTGGTTTATTTTTGTTCC
ACAAGTTAAATAAATCATAAAACTTGATGTGTTATCTCTTATATCTCACTCCCACTAT
TACCCCTTTATTTTCAAACAGGGAAACAGTCTTCAAGTTCCACTTGGTAAAAAATGT
GAACCCCTTGTATATAGAGTTTGGCTCACAGTGTAAAGGGCCTCAGTGATTCACATT
TTCCAGATTAGGAATCTGATGCTCAAAGAAGTTAAATGGCATAGTTGGGGTGACAC
AGCTGTCTAGTGGGAGGCCAGCCTTCTATATTTTAGCCAGCGTTCTTTCCTGCGGGCC
AGGTCATGAGGAGTATGCAGACTCTAAGAGGGAGCAAAAGTATCTGAAGGATTTAA
TATTTTAGCAAGGAATAGATATACAATCATCCCTTGGTCTCCCTGGGGGATTGGTTT
CAGGACCCCTTCTTGGACACCAAATCTATGGATATTTAAGTCCCTTCTATAAAATGG
TATAGTATTTGCATATAACCTATCCACATCCTCCTGTATACTTTAAATCATTTCTAGA
TTACTTGTAATACCTAATACAATGTAAATGCTATGCAAATAGTTGTTATTGTTTAAGG
AATAATGACAAGAAAAAAAAGTCTGTACATGCTCAGTAAAGACACAACCATCCCTT
TTTTTCCCCAGTGTTTTTGATCCATGGTTTGCTGAATCCACAGATGTGGAGCCCCTGG
ATACGGAAGGCCCGCTGTACTTTGAATGACAAATAACAGATTTAAAATTTTCAAGGC
ATAGTTTTATACCTGA
LENGTH: 1675
TYPE: DNA
ORGANISM: HOMO SAPIENS
FEATURE: BETA-2-MICROGLOBULIN (B2M) CODING SEQUENCE
OTHER INFORMATION: SaCas9 guide RNA sequence is underlined.
SEQ ID NO 2
ATTCCTGAAGCTGACAGCATTCGGGCCGAGATGTCTCGCTCCGTGGCCTTAGCTGTG
CTCGCGCTACTCTCTCTTTCTGGCCTGGAGGCTATCCAGCGTACTCCAAAGATTCAG
GTTTACTCACGTCATCCAGCAGAGAATGGAAAGTCAAATTTCCTGAATTGCTATGTG
TCTGGGTTTCATCCATCCGACATTGAAGTTGACTTACTGAAGAATGGAGAGAGAATT
GAAAAAGTGGAGCATTCAGACTTGTCTTTCAGCAAGGACTGGTCTTTCTATCTCTTG
TACTACACTGAATTCACCCCCACTGAAAAAGATGAGTATGCCTGCCGTGTGAACCAT
GTGACTTTGTCACAGCCCAAGATAGTTAAGTGGGATCGAGACATGTAAGCAGCATC
ATGGAGGTTTGAAGATGCCGCATTTGGATTGGATGAATTCCAAATTCTGCTTGCTTG
CTTTTTAATATTGATATGCTTATACACTTACACTTTATGCACAAAATGTAGGGTTATA
ATAATGTTAACATGGACATGATCTTCTTTATAATTCTACTTTGAGTGCTGTCTCCATG
TTTGATGTATCTGAGCAGGTTGCTCCACAGGTAGCTCTAGGAGGGCTGGCAACTTAG
AGGTGGGGAGCAGAGAATTCTCTTATCCAACATCAACATCTTGGTCAGATTTGAACT
CTTCAATCTCTTGCACTCAAAGCTTGTTAAGATAGTTAAGCGTGCATAAGTTAACTTC
CAATTTACATACTCTGCTTAGAATTTGGGGGAAAATTTAGAAATATAATTGACAGGA
TTATTGGAAATTTGTTATAATGAATGAAACATTTTGTCATATAAGATTCATATTTACT
TCTTATACATTTGATAAAGTAAGGCATGGTTGTGGTTAATCTGGTTTATTTTTGTTCC
ACAAGTTAAATAAATCATAAAACTTGATGTGTTATCTCTTATATCTCACTCCCACTAT
TACCCCTTTATTTTCAAACAGGGAAACAGTCTTCAAGTTCCACTTGGTAAAAAATGT
GAACCCCTTGTATATAGAGTTTGGCTCACAGTGTAAAGGGCCTCAGTGATTCACATT
TTCCAGATTAGGAATCTGATGCTCAAAGAAGTTAAATGGCATAGTTGGGGTGACAC
AGCTGTCTAGTGGGAGGCCAGCCTTCTATATTTTAGCCAGCGTTCTTTCCTGCGGGCC
AGGTCATGAGGAGTATGCAGACTCTAAGAGGGAGCAAAAGTATCTGAAGGATTTAA
TATTTTAGCAAGGAATAGATATACAATCATCCCTTGGTCTCCCTGGGGGATTGGTTT
CAGGACCCCTTCTTGGACACCAAATCTATGGATATTTAAGTCCCTTCTATAAAATGG
TATAGTATTTGCATATAACCTATCCACATCCTCCTGTATACTTTAAATCATTTCTAGA
TTACTTGTAATACCTAATACAATGTAAATGCTATGCAAATAGTTGTTATTGTTTAAGG
AATAATGACAAGAAAAAAAAGTCTGTACATGCTCAGTAAAGACACAACCATCCCTT
TTTTTCCCCAGTGTTTTTGATCCATGGTTTGCTGAATCCACAGATGTGGAGCCCCTGG
ATACGGAAGGCCCGCTGTACTTTGAATGACAAATAACAGATTTAAAATTTTCAAGGC
ATAGTTTTATACCTGA
LENGTH: 1675
TYPE: DNA
ORGANISM: HOMO SAPIENS
FEATURE: BETA-2-MICROGLOBULIN (B2M) CODING SEQUENCE
OTHER INFORMATION: SaCas9 guide RNA sequence is underlined.
SEQ ID NO 3
TTTTGAAACCCTTCAAAGGCAGAGACTTGTCCAGCCTAACCTGCCTGCTGCTCCTAG
CTCCTGAGGCTCAGGGCCCTTGGCTTCTGTCCGCTCTGCTCAGGGCCCTCCAGCGTG
GCCACTGCTCAGCCATGCTCCTGCTGCTCGTCCCAGTGCTCGAGGTGATTTTTACCCT
GGGAGGAACCAGAGCCCAGTCGGTGACCCAGCTTGGCAGCCACGTCTCTGTCTCTG
AAGGAGCCCTGGTTCTGCTGAGGTGCAACTACTCATCGTCTGTTCCACCATATCTCTT
CTGGTATGTGCAATACCCCAACCAAGGACTCCAGCTTCTCCTGAAGTACACATCAGC
GGCCACCCTGGTTAAAGGCATCAACGGTTTTGAGGCTGAATTTAAGAAGAGTGAAA
CCTCCTTCCACCTGACGAAACCCTCAGCCCATATGAGCGACGCGGCTGAGTACTTCT
GTGCTGTGAGTGATCTCGAACCGAACAGCAGTGCTTCCAAGATAATCTTTGGATCAG
GGACCAGACTCAGCATCCGGCCAAATATCCAGAACCCTGACCCTGCCGTGTACCAG
CTGAGAGACTCTAAATCCAGTGACAAGTCTGTCTGCCTATTCACCGATTTTGATTCTC
AAACAAATGTGTCACAAAGTAAGGATTCTGATGTGTATATCACAGACAAAACTGTG
CTAGACATGAGGTCTATGGACTTCAAGAGCAACAGTGCTGTGGCCTGGAGCAACAA
ATCTGACTTTGCATGTGCAAACGCCTTCAACAACAGCATTATTCCAGAAGACACCTT
CTTCCCCAGCCCAGAAAGTTCCTGTGATGTCAAGCTGGTCGAGAAAAGCTTTGAAAC
AGATACGAACCTAAACTTTCAAAACCTGTCAGTGATTGGGTTCCGAATCCTCCTCCT
GAAAGTGGCCGGGTTTAATCTGCTCATGACGCTGCGGCTGTGGTCCAGCTGAGATCT
GCAAGATTGTAAGACAGCCTGTGCTCCCTCGCTCCTTCCTCTGCATTGCCCCTCTTCT
CCCTCTCCAAACAGAGGGAACTCTCCTACCCCCAAGGAGGTGAAAGCTGCTACCAC
CTCTGTGCCCCCCCGGTAATGCCACCAACTGGATCCTACCCGAATTTATGATTAAGA
TTGCTGAAGAGCTGCCAAACACTGCTGCCACCCCCTCTGTTCCCTTATTGCTGCTTGT
CACTGCCTGACATTCACGGCAGAGGCAAGGCTGCTGCAGCCTCCCCTGGCTGTGCAC
ATTCCCTCCTGCTCCCCAGAGACTGCCTCCGCCATCCCACAGATGATGGATCTTCAG
TGGGTTCTCTTGGGCTCTAGGTCCTGGAGAATGTTGTGAGGGGTTTATTTTTTTTTAA
TAGTGTTCATAAAGAAATACATAGTATTCTTCTTCTCAAGACGTGGGGGGAAATTAT
CTCATTATCGAGGCCCTGCTATGCTGTGTGTCTGGGCGTGTTGTATGTCCTGCTGCCG
ATGCCTTCATTAAAATGATTTGGAA
LENGTH: 1508
TYPE: DNA
ORGANISM: HOMO SAPIENS
FEATURE: T CELL RECEPTOR ALPHA CONSTANT (TRAC) CODING SEQUENCE
OTHER INFORMATION: SaCas9 guide RNA sequence is underlined.
SEQ ID NO 4
GGGCTCCAGGCTGCTCTGTTGGGTGCTGCTTTGTCTCCTGGGAGCAGGCCCAGTAAA
GGCTGGAGTCACTCAAACTCCAAGATATCTGATCAAAACGAGAGGACAGCAAGTGA
CACTGAGCTGCTCCCCTATCTCTGGGCATAGGAGTGTATCCTGGTACCAACAGACCC
CAGGACAGGGCCTTCAGTTCCTCTTTGAATACTTCAGTGAGACACAGAGAAACAAA
GGAAACTTCCCTGGTCGATTCTCAGGGCGCCAGTTCTCTAACTCTCGCTCTGAGATG
AATGTGAGCACCTTGGAGCTGGGGGACTCGGCCCTTTATCTTTGCGCCAGCAGCCGG
ACAGGGGGCGTAAAGAATTCACCCCTCCACTTTGGGAACGGGACCAGGCTCACTGT
GACAGAGGACCTGAACAAGGTGTTCCCACCCGAGGTCGCTGTGTTTGAGCCATCAG
AAGCAGAGATCTCCCACACCCAAAAGGCCACACTGGTGTGCCTGGCCACAGGCTTC
TTCCCTGACCACGTGGAGCTGAGCTGGTGGGTGAATGGGAAGGAGGTGCACAGTGG
GGTCAGCACGGACCCGCAGCCCCTCAAGGAGCAGCCCGCCCTCAATGACTCCAGAT
ACTGCCTGAGCAGCCGCCTGAGGGTCTCGGCCACCTTCTGGCAGAACCCCCGCAACC
ACTTCCGCTGTCAAGTCCAGTTCTACGGGCTCTCGGAGAATGACGAGTGGACCCAGG
ATAGGGCCAAACCCGTCACCCAGATCGTCAGCGCCGAGGCCTGGGGTAGAGCAGAC
TGTGGCTTTACCTCGGTGTCCTACCAGCAAGGGGTCCTGTCTGCCACCATCCTCTATG
AGATCCTGCTAGGGAAGGCCACCCTGTATGCTGTGCTGGTCAGCGCCCTTGTGTTGA
TGGCCATGGTCAAGAGAAAGGATTTCTGAAGGCAGCCCTGGAAGTGGAGTTAGGAG
CTTCTAACCCGTCATGGTTTCAATACACATTCTTCTTTTGCCAGCGCTTCTGAAGAGC
TGCTCTCACCTCTCTGCATCCCAATAGATATCCCCCTATGTGCATGCACACCTGCACA
CTCACGGCTGAAATCTCCCTAACCCAGGGGGACCTTAGCATGCCTAAGTGACTAAAC
CAATAAAAATGTTCTGGTCTGGCCTGAAAAAAAAAAAAAAAAAAAAAAAAAAAAA
AAAAAAAAAAAAAAAAAAAAAAAAAAAAAAAA
LENGTH: 1221
TYPE: DNA
ORGANISM: HOMO SAPIENS
FEATURE: T CELL RECEPTOR BETA CONSTANT (TRBC) CODING SEQUENCE
OTHER INFORMATION: Sequence targeted by TevSaCas9 highlighted. I-TevI site is
underlined, Cas9 guide RNA sequence is double underlined and PAM sequence is lowercase.
SEQ ID NO 5
GATGGCCGTCATGGCGCCCCGAACCCTCCTCCTGCTACTCTCGGGGGCCCTGGCCCT
GACCCAGACCTGGGCGGGCTCCCACTCCATGAGGTATTTCTTCACATCCGTGTCCCG
GCCCGGCCGCGGGGAGCCCCGCTTCATCGCCGTGGGCTACGTGGACGACACGCAGT
TCGTGCGGTTCGACAGCGACGCCGCGAGCCAGAAGATGGAGCCGCGGGCGCCGTGG
ATAGAGCAGGAGGGGCCGGAGTATTGGGACCAGGAGACACGGAATATGAAGGCCC
ACTCACAGACTGACCGAGCGAACCTGGGGACCCTGCGCGGCTACTACAACCAGAGC
GAGGACGGTTCTCACACCATCCAGATAATGTATGGCTGCGACGTGGGGCCGGACGG
GCGCTTCCTCCGCGGGTACCGGCAGGACGCCTACGACGGCAAGGATTACATCGCCC
TGAACGAGGACCTGCGCTCTTGGACCGCGGCGGACATGGCAGCTCAGATCACCAAG
CGCAAGTGGGAGGCGGTCCATGCGGCGGAGCAGCGGAGAGTCTACCTGGAGGGCC
GGTGCGTGGACGGGCTCCGCAGATACCTGGAGAACGGGAAGGAGACGCTGCAGCG
CACGGACCCCCCCAAGACACATATGACCCACCACCCCATCTCTGACCATGAGGCCA
CCCTGAGGTGCTGGGCCCTGGGCTTCTACCCTGCGGAGATCACACTGACCTGGCAGC
GGGATGGGGAGGACCAGACCCAGGACACGGAGCTCGTGGAGACCAGGCCTGCAGG
GGATGGAACCTTCCAGAAGTGGGCGGCTGTGGTGGTGCCTTCTGGAGAGGAGCAGA
GATACACCTGCCATGTGCAGCATGAGGGTCTGCCCAAGCCCCTCACCCTGAGATGG
GAGCTGTCTTCCCAGCCCACCATCCCCATCGTGGGCATCATTGCTGGCCTGGTTCTCC
TTGGAGCTGTGATCACTGGAGCTGTGGTCGCTGCCGTGATGTGGAGGAGGAAGAGC
TCAGATAGAAAAGGAGGGAGTTACACTCAGGCTGCAAGCAGTGACAGTGCCCAGGG
CTCTGATGTGTCTCTCACAGCTTGTAAAGTGTGAGACAGCTGCCTTGTGTGGGACTG
AGAGGCAAGAGTTGTTCCTGCCCTTCCCTTTGTGACTTGAAGAACCCTGACTTTGTTT
CTGCAAAGGCACCTGCATGTGTCTGTGTTCGTGTAGGCATAATGTGAGGAGGTGGGG
AGAGCACCCCACCCCCATGTCCACCATGACCCTCTTCCCACGCTGACCTGTGCTCCC
TCTCCAATCATCTTTCCTGTTCCAGAGAGGTGGGGCTGAGGTGTCTCCATCTCTGTCT
CAACTTCATGGTGCACTGAGCTGTAACTTCTTCCTTCCCTATTAAAATTAGAACCTGA
GTATAAAAAAAAAAAAAAAAAAAA
LENGTH: 1434
TYPE: DNA
ORGANISM: HOMO SAPIENS
FEATURE: MAJOR HISTOCOMPATIBILITY COMPLEX, CLASS I, A (HLA-A) CODING
SEQUENCE
OTHER INFORMATION: Sequence targeted by TevSaCas9 highlighted. I-TevI site is
underlined, Cas9 guide RNA sequence is double underlined and PAM sequence is lowercase.
SEQ ID NO 6
GTCAGCGCCCCCCGCCCGGCGTCTCCCGGGGCCAGGTCCACCCTCTGCTGCGCCACC
TGGGGCATCCTCCTTCCCCGTTGCCAGTCTCGATCCGCCCCGTCGTTCCTGGCCCTGG
GCTTTGCCACCCTATGCTGACACCCCGTCCCAGTCCCCCTTACCATTCCCCTTCGACC
ACCCCACTTCCGAATTGGAGCCGCTTCAACTGGCCCTGGGCTTAGCCACTCTGTGCT
GACCACTCTGCCCCAGGCCTCCTTACCATTCCCCTTCGACCTACTCTCTTCCGCATTG
GAGTCGCTTTAACTGGCCCTGGCTTTGGCAGCCTGTGCTGACCCATGCAGTCCTCCTT
ACCATCCCTCCCTCGACTTCCCCTCTTCCGATGTTGAGCCCCTCCAGCCGGTCCTGGA
CTTTGTCTCCTTCCCTGCCCTGCCCTCTCCTGAACCTGAGCCAGCTCCCATAGCTCAG
TCTGGTCTATCTGCCTGGCCCTGGCCATTGTCACTTTGCGCTGCCCTCCTCTCGCCCC
CGAGTGCCCTTGCTGTGCCGCCGGAACTCTGCCCTCTAACGCTGCCGTCTCTCTCCTG
AGTCCGGACCACTTTGAGCTCTACTGGCTTCTGCGCCGCCTCTGGCCCACTGTTTCCC
CTTCCCAGGCAGGTCCTGCTTTCTCTGACCTGCATTCTCTCCCCTGGGCCTGTGCCGC
TTTCTGTCTGCAGCTTGTGGCCTGGGTCACCTCTACGGCTGGCCCAGATCCTTCCCTG
CCGCCTCCTTCAGGTTCCGTCTTCCTCCACTCCCTCTTCCCCTTGCTCTCTGCTGTGTT
GCTGCCCAAGGATGCTCTTTCCGGAGCACTTCCTTCTCGGCGCTGCACCACGTGATG
TCCTCTGAGCGGATCCTCCCCGTGTCTGGGTCCTCTCCGGGCATCTCTCCTCCCTCAC
CCAACCCCATGCCGTCTTCACTCGCTGGGTTCCCTTTTCCTTCTCCTTCTGGGGCCTG
TGCCATCTCTCGTTTCTTAGGATGGCCTTCTCCGACGGATGTCTCCCTTGCGTCCCGC
CTCCCCTTCTTGTAGGCCTGCATCATCACCGTTTTTCTGGACAACCCCAAAGTACCCC
GTCTCCCTGGCTTTAGCCACCTCTCCATCCTCTTGCTTTCTTTGCCTGGACACCCCGTT
CTCCTGTGGATTCGGGTCACCTCTCACTCCTTTCATTTGGGCAGCTCCCCTACCCCCC
TTACCTCTCTAGTCTGTGCTAGCTCTTCCAGCCCCCTGTCATGGCATCTTCCAGGGGT
CCGAGAGCTCAGCTAGTCTTCTTCCTCCAACCCGGGCCCCTATGTCCACTTCAGGAC
AGCATGTTTGCTGCCTCCAGGGATCCTGTGTCCCCGAGCTGGGACCACCTTATATTC
CCAGGGCCGGTTAATGTGGCTCTGGTTCTGGGTACTTTTATCTGTCCCCTCCACCCCA
CAGTGGGGCCACTAGGGACAGGATTGGTGACAGAAAAGCCCCATCCTTAGGCCTCC
TCCTTCCTAGTCTCCTGATATTGGGTCTAACCCCCACCTCCTGTTAGGCAGATTCCTT
ATCTGGTGACACACCCCCATTTCCTGGAGCCATCTCTCTCCTTGCCAGAACCTCTAA
GGTTTGCTTACGATGGAGCCAGAGAGGATCCTGGGAGGGAGAGCTTGGCAGGGGGT
GGGAGGGAAGGGGGGGATGCGTGACCTGCCCGGTTCTCAGTGGCCACCCTGCGCTA
CCCTCTCCCAGAACCTGAGCTGCTCTGACGCGGCCGTCTGGTGCGTTTCACTGATCC
TGGTGCTGCAGCTTCCTTACACTTCCCAAGAGGAGAAGCAGTTTGGAAAAACAAAA
TCAGAATAAGTTGGTCCTGAGTTCTAACTTTGGCTCTTCACCTTTCTAGTCCCCAATT
TATATTGTTCCTCCGTGCGTCAGTTTTACCTGTGAGATAAGGCCAGTAGCCAGCCCC
GTCCTGGCAGGGCTGTGGTGAGGAGGGGGGTGTCCGTGTGGAAAACTCCCTTTGTG
AGAATGGTGCGTCCTAGGTGTTCACCAGGTCGTGGCCGCCTCTACTCCCTTTCTCTTT
CTCCATCCTTCTTTCCTTAAAGAGTCCCCAGTGCTATCTGGGACATATTCCTCCGCCC
AGAGCAGGGTCCCGCTTCCCTAAGGCCCTGCTCTGGGCTTCTGGGTTTGAGTCCTTG
GCAAGCCCAGGAGAGGCGCTCAGGCTTCCCTGTCCCCCTTCCTCGTCCACCATCTCA
TGCCCCTGGCTCTCCTGCCCCTTCCCTACAGGGGTTCCTGGCTCTGCTCTTCAGACTG
AGCCCCGTTCCCCTGCATCCCCGTTCCCCTGCATCCCCCTTCCCCTGCATCCCCCAGA
GGCCCCAGGCCACCTACTTGGCCTGGACCCCACGAGAGGCCACCCCAGCCCTGTCTA
CCAGGCTGCCTTTTGGGTGGATTCTCCTCCAACTGTGGGGTGACTGCTTGGCAAACT
CACTCTTCGGGGTATCCCAGGAGGCCTGGAGCATTGGGGTGGGCTGGGGTTCAGAG
AGGAGGGATTCCCTTCTCAGGTTACGTGGCCAAGAAGCAGGGGAGCTGGGTTTGGG
TCAGGTCTGGGTGTGGGGTGACCAGCTTATGCTGTTTGCCCAGGACAGCCTAGTTTT
AGCACTGAAACCCTCAGTCCTAGGAAAACAGGGATGGTTGGTCACTGTCTCTGGGTG
ACTCTTGATTCCCGGCCAGTTTCTCCACCTGGGGCTGTGTTTCTCGTCCTGCATCCTT
CTCCAGGCAGGTCCCCAAGCATCGCCCCCCTGCTGTGGCTGTTCCCAAGTTCTTAGG
GTACCCCACGTGGGTTTATCAACCACTTGGTGAGGCTGGTACCCTGCCCCCATTCCT
GCACCCCAATTGCCTTAGTGGCTAGGGGGTTGGGGGCTAGAGTAGGAGGGGCTGGA
GCCAGGATTCTTAGGGCTGAACAGAGAAGAGCTGGGGGCCTGGGCTCCTGGGTTTG
AGAGAGGAGGGGCTGGGGCCTGGACTCCTGGGTCCGAGGGAGGAGGGGCTGGGGC
CTGGACTCCTGGGTCTGAGGGTGGAGGGACTGGGGGCCTGGACTCCTGGGTCCGAG
GGAGGAGGGGCTGGGGCCTGGACTCGTGGGTCTGAGGGAGGAGGGGCTGGGGGCC
TGGACTTCTGGGTCTTAGGGAGGCGGGGCTGGGCCTGGACCCCTGGGTCTGAATGG
GGAGAGGCTGGGGGCCTGGACTCCTTCATCTGAGGGCGGAAGGGCTGGGGCCTGGC
CTCCTGGGTTGAATGGGGAGGGGTTGGGCCTGGACTCTGGAGTCCCTGGTGCCCAGG
CCTCAGGCATCTTTCACAGGGATGCCTGTACTGGGCAGGTCCTTGAAAGGGAAAGG
CCCATTGCTCTCCTTGCCCCCCTCCCCTATCGCCATGACAACTGGGTGGAAATAAAC
GAGCCGAGTTCATCCCGTTCCCAGGGCACGTGCGGCCCCTTCACAGCCCGAGTTTCC
ATGACCTCATGCTCTTGGCCCTCGTAGCTCCCTCCCGCCTCCTCCAGATGGGCAGCTT
TGGAGAGGTGAGGGACTTGGGGGGTAATTTATCCCGTGGATCTAGGAGTTTAGCTTC
ACTCCTTCCTCAGCTCCAGTTCAGGTCCCGGAGCCCACCCAGTGTCCACAAGGCCTG
GGGCAAGTCCCTCCTCCGACCCCCTGGACTTCGGCTTTTGTCCCCCCAAGTTTTGGAC
CCCTAAGGGAAGAATGAGAAACGGTGGCCCGTGTCAGCCCCTGGCTGCAGGGCCCC
GTGCAGAGGGGGCCTCAGTGAACTGGAGTGTGACAGCCTGGGGCCCAGGCACACAG
GTGTGCAGCTGTCTCACCCCTCTGGGAGTCCCGCCCAGGCCCCTGAGTCTGTCCCAG
CACAGGGTGGCCTTCCTCCACCCTGCATAGCCCTGGGCCCACGGCTTCGTTCCTGCA
GAGTATCTGCTGGGGTGGTTTCCGAGCTTGACCCTTGGAAGGACCTGGCTGGGTTTA
AGGCAGGAGGGGCTGGGGGCCAGGACTCCTGGCTCTGAAGGAGGAGGGGCTGGAA
CCTCTTCCCTAGTCTGAGCACTGGAAGCGCCACCTGTGGGTGGTGACGGGGGTTTTG
CCGTGTCTAACAGGTACCATGTGGGGTTCCCGCACCCAGATGAGAAGCCCCCTCCCT
TCCCCGTTCACTTCCTGTTTGCAGATAGCCAGGAGTCCTTTCGTGGTTTCCACTGAGC
ACTGAAGGCCTGGCCGGCCTGACCACTGGGCAACCAGGCGTATCTTAAACAGCCAG
TGGCCAGAGGCTGTTGGGTCATTTTCCCCACTGTCCTAGCACCGTGTCCCTGGATCTG
TTTTCGTGGCTCCCTCTGGAGTCCCGACTTGCTGGGACACCGTGGCTGGGGTAGGTG
CGGCTGACGGCTGTTTCCCACCCCCAG
LENGTH: 4427
TYPE: DNA
ORGANISM: HOMO SAPIENS
FEATURE: ADENO-ASSOCIATED VIRUS INTEGRATION SITE 1 (AAVS1) NON-
CODING SEQUENCE
OTHER INFORMATION: Sequence targeted by TevSaCas9 highlighted. I-TevI site is
underlined, Cas9 guide RNA sequence is double underlined and PAM sequence is lowercase.
SEQ ID NO 7
5′-
GGGCAGAGCGCACATCGCCCACAGTCCCCGAGAAGTTGGGGGGAGGGGTCGGCAAT
TGAACCGGTGCCTAGAGAAGGTGGCGCGGGGTAAACTGGGAAAGTGATGTCGTGTA
CTGGCTCCGCCTTTTTCCCGAGGGTGGGGGAGAACCGTATATAAGTGCAGTAGTCGC
CGTGAACGTTCTTTTTCGCAACGGGTTTGCCGCCAGAACACAGGTGTCGTGACGCGG
GATCCGCCACCATGCTTCTCCTGGTGACAAGCCTTCTGCTCTGTGAGTTACCACACC
CAGCATTCCTCCTGATCCCAGACATCCAGATGACACAGACTACATCCTCCCTGTCTG
CCTCTCTGGGAGACAGAGTCACCATCAGTTGCAGGGCAAGTCAGGACATTAGTAAA
TATTTAAATTGGTATCAGCAGAAACCAGATGGAACTGTTAAACTCCTGATCTACCAT
ACATCAAGATTACACTCAGGAGTCCCATCAAGGTTCAGTGGCAGTGGGTCTGGAAC
AGATTATTCTCTCACCATTAGCAACCTGGAGCAAGAAGATATTGCCACTTACTTTTG
CCAACAGGGTAATACGCTTCCGTACACGTTCGGAGGGGGGACTAAGTTGGAAATAA
CAGGCTCCACCTCTGGATCCGGCAAGCCCGGATCTGGCGAGGGATCCACCAAGGGC
GAGGTGAAACTGCAGGAGTCAGGACCTGGCCTGGTGGCGCCCTCACAGAGCCTGTC
CGTCACATGCACTGTCTCAGGGGTCTCATTACCCGACTATGGTGTAAGCTGGATTCG
CCAGCCTCCACGAAAGGGTCTGGAGTGGCTGGGAGTAATATGGGGTAGTGAAACCA
CATACTATAATTCAGCTCTCAAATCCAGACTGACCATCATCAAGGACAACTCCAAGA
GCCAAGTTTTCTTAAAAATGAACAGTCTGCAAACTGATGACACAGCCATTTACTACT
GTGCCAAACATTATTACTACGGTGGTAGCTATGCTATGGACTACTGGGGTCAAGGAA
CCTCAGTCACCGTCTCCTCAGCGGCCGCAATTGAAGTTATGTATCCTCCTCCTTACCT
AGACAATGAGAAGAGCAATGGAACCATTATCCATGTGAAAGGGAAACACCTTTGTC
CAAGTCCCCTATTTCCCGGACCTTCTAAGCCCTTTTGGGTGCTGGTGGTGGTTGGGG
GAGTCCTGGCTTGCTATAGCTTGCTAGTAACAGTGGCCTTTATTATTTTCTGGGTGAG
GAGTAAGAGGAGCAGGCTCCTGCACAGTGACTACATGAACATGACTCCCCGCCGCC
CCGGGCCCACCCGCAAGCATTACCAGCCCTATGCCCCACCACGCGACTTCGCAGCCT
ATCGCTCCAGAGTGAAGTTCAGCAGGAGCGCAGACGCCCCCGCGTACCAGCAGGGC
CAGAACCAGCTCTATAACGAGCTCAATCTAGGACGAAGAGAGGAGTACGATGTTTT
GGACAAGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGCCTACAG
TGAGATTGGGATGAAAGGCGAGCGCCGGAGGGGCAAGGGGCACGATGGCCTTTACC
AGGGTCTCAGTACAGCCACCAAGGACACCTACGACGCCCTTCACATGCAGGCCCTG
CCCCCTCGCTAA-3′
3′-
CCCCCGTCTCGCGTGTAGCGGGTGTCAGGGGCTCTTCAACCCCCCTCCCCAGCCGTT
AACTTGGCCACGGATCTCTTCCACCGCGCCCCATTTGACCCTTTCACTACAGCACAT
GACCGAGGCGGAAAAAGGGCTCCCACCCCCTCTTGGCATATATTCACGTCATCAGC
GGCACTTGCAAGAAAAAGCGTTGCCCAAACGGCGGTCTTGTGTCCACAGCACTGCG
CCCTAGGCGGTGGTACGAAGAGGACCACTGTTCGGAAGACGAGACACTCAATGGTG
TGGGTCGTAAGGAGGACTAGGGTCTGTAGGTCTACTGTGTCTGATGTAGGAGGGAC
AGACGGAGAGACCCTCTGTCTCAGTGGTAGTCAACGTCCCGTTCAGTCCTGTAATCA
TTTATAAATTTAACCATAGTCGTCTTTGGTCTACCTTGACAATTTGAGGACTAGATGG
TATGTAGTTCTAATGTGAGTCCTCAGGGTAGTTCCAAGTCACCGTCACCCAGACCTT
GTCTAATAAGAGAGTGGTAATCGTTGGACCTCGTTCTTCTATAACGGTGAATGAAAA
CGGTTGTCCCATTATGCGAAGGCATGTGCAAGCCTCCCCCCTGATTCAACCTTTATTG
TCCGAGGTGGAGACCTAGGCCGTTCGGGCCTAGACCGCTCCCTAGGTGGTTCCCGCT
CCACTTTGACGTCCTCAGTCCTGGACCGGACCACCGCGGGAGTGTCTCGGACAGGCA
GTGTACGTGACAGAGTCCCCAGAGTAATGGGCTGATACCACATTCGACCTAAGCGG
TCGGAGGTGCTTTCCCAGACCTCACCGACCCTCATTATACCCCATCACTTTGGTGTAT
GATATTAAGTCGAGAGTTTAGGTCTGACTGGTAGTAGTTCCTGTTGAGGTTCTCGGT
TCAAAAGAATTTTTACTTGTCAGACGTTTGACTACTGTGTCGGTAAATGATGACACG
GTTTGTAATAATGATGCCACCATCGATACGATACCTGATGACCCCAGTTCCTTGGAG
TCAGTGGCAGAGGAGTCGCCGGCGTTAACTTCAATACATAGGAGGAGGAATGGATC
TGTTACTCTTCTCGTTACCTTGGTAATAGGTACACTTTCCCTTTGTGGAAACAGGTTC
AGGGGATAAAGGGCCTGGAAGATTCGGGAAAACCCACGACCACCACCAACCCCCTC
AGGACCGAACGATATCGAACGATCATTGTCACCGGAAATAATAAAAGACCCACTCC
TCATTCTCCTCGTCCGAGGACGTGTCACTGATGTACTTGTACTGAGGGGCGGCGGGG
CCCGGGTGGGCGTTCGTAATGGTCGGGATACGGGGTGGTGCGCTGAAGCGTCGGAT
AGCGAGGTCTCACTTCAAGTCGTCCTCGCGTCTGCGGGGGCGCATGGTCGTCCCGGT
CTTGGTCGAGATATTGCTCGAGTTAGATCCTGCTTCTCTCCTCATGCTACAAAACCTG
TTCTCTGCACCGGCCCTGGGACTCTACCCCCCTTTCGGCTCTTCCTTCTTGGGAGTCC
TTCCGGACATGTTACTTGACGTCTTTCTATTCTACCGCCTCCGGATGTCACTCTAACC
CTACTTTCCGCTCGCGGCCTCCCCGTTCCCCGTGCTACCGGAAATGGTCCCAGAGTC
ATGTCGGTGGTTCCTGTGGATGCTGCGGGAAGTGTACGTCCGGGACGGGGGAGCGA
TT-5′
LENGTH: 1707
TYPE: DOUBLE-STRAND DNA
ORGANISM: ARTIFICAL
FEATURE: INSERTS A CHIMERIC ANTIGEN RECEPTOR TARGTING CD19 INTO THE
AAVS1 SITE; DNA ORIENTATION IS INDICATED BY 5′ or 3′; 2 NUCLEOTIDE
OVERHANG INDICATED BY UNDERLINE
OTHER INFORMATION:
SEQ ID NO 8
MGKSGIYQIKNTLNNKVYVGSAKDFEKRWKRHFKDLEKGCHSSIKLQRSFNKHGNVFE
CSILEEIPYEKDLIIERENFWIKELNSKINGYNIA
LENGTH: 93
TYPE: AMINO ACID
ORGANISM: ARTIFICIAL
FEATURE: I-TEVI DOMAIN
OTHER INFORMATION:
SEQ ID NO 9
DATFGDTCSTHPLKEEIIKKRSETFKAKMLKLGPDGRKALYSKPGSKNGRWNPETHKFC
KCGVRIQTSAYTCSKCRNGGSGGS
LENGTH: 83
TYPE: AMINO ACID
ORGANISM: ARTIFICAL
FEATURE: LINKER DOMAIN
OTHER INFORMATION: V117F; mutation underlined
SEQ ID NO 10
DATFGDTCSTHPLKEEIIKKRSETVKAKMLKLGPDGRKALYSRPGSKSGRWNPETHKFC
KCGVRIQTSAYTCSKCRNGGSGGS
LENGTH: 83
TYPE: AMINO ACID
ORGANISM: ARTIFICAL
FEATURE: LINKER DOMAIN
OTHER INFORMATION: K135R/N140S; mutations underlined
SEQ ID NO 11
DATFGDTCSTHPLKEEIIKKRSETFKAKMLKLGPDGRKALYSRPGSKSGRWNPETHKFC
KCGVRIQTSAYTCSKCRNGGSGGS
LENGTH: 83
TYPE: AMINO ACID
ORGANISM: ARTIFICAL
FEATURE: LINKER DOMAIN
OTHER INFORMATION: V117F/K135R/N140S; mutations underlined
SEQ ID NO 12
DATFGDTCSTHPLKEEIIKKRSETVKAKMLKLGPDGRKALYSRPGSKSGRWNPETHKFC
KCGVRIQTSAYTCSKCRNGGSGGTGGS
LENGTH: 86
TYPE: AMINO ACID
ORGANISM: ARTIFICIAL
FEATURE: LINKER DOMAIN VARIANT
OTHER INFORMATION:
SEQ ID NO 13
DATFGDTCSTHPLKEEIIKKRSETVKAKMLKLGPDGRKALYSRPGSKSGRWNPETHKFC
KCGVRIQTSAYTCSKCRNGGGGSGGGGS
LENGTH: 87
TYPE: AMINO ACID
ORGANISM: ARTIFICIAL
FEATURE: LINKER DOMAIN VARIANT
OTHER INFORMATION:
SEQ ID NO 14
DATFGDTCSTHPLKEEIIKKRSETVKAKMLKLGPDGRKALYSRPGSKSGRWNPETHKFC
KCGVRIQTSAYTCSKCRNKESGSVSSEQLAQFRSLD
LENGTH: 95
TYPE: AMINO ACID
ORGANISM: ARTIFICIAL
FEATURE: LINKER DOMAIN VARIANT
OTHER INFORMATION:
SEQ ID NO 15
MKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,053
TYPE: AMINO ACID
ORGANISM: STAPHYLOCOCCUS AUREUS
FEATURE:
OTHER INFORMATION:
SEQ ID NO 16
MKRNYILGLEIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,053
TYPE: AMINO ACID
ORGANISM: ARTIFICAL
FEATURE: SACAS9 D10E MUTANT
OTHER INFORMATION: Mutation underlined
SEQ ID NO 17
CGCUACUCUCUCUUUCUGGCC
LENGTH: 21
TYPE: RNA
ORGANISM: ARTIFICAL
FEATURE: TARGETS B2M GENE
OTHER INFORMATION:
SEQ ID NO 18
AGCGCGAGCACAGCUAAGGCC
LENGTH: 21
TYPE: RNA
ORGANISM: ARTIFICAL
FEATURE: TARGETS B2M GENE
OTHER INFORMATION:
SEQ ID NO 19
UUUUGUCUGUGAUAUACACAU
LENGTH: 21
TYPE: RNA
ORGANISM: ARTIFICAL
FEATURE: TARGETS TRAC GENE
OTHER INFORMATION:
SEQ ID NO 20
CUUGACAGCGGAAGUGGUUGC
LENGTH: 21
TYPE: RNA
ORGANISM: ARTIFICAL
FEATURE: TARGETS TRBC GENE
OTHER INFORMATION:
SEQ ID NO 21
UGGGUCAGGGCCAGGGCCCCC
LENGTH: 21
TYPE: RNA
ORGANISM: ARTIFICAL
FEATURE: TARGETS HLA-A GENE
OTHER INFORMATION:
SEQ ID NO 22
CCCUGCUCUGGGCUUCUGGGU
LENGTH: 21
TYPE: RNA
ORGANISM: ARTIFICAL
FEATURE: TARGETS AAVS1 SITE
OTHER INFORMATION:
SEQ ID NO 23
5′-
GGGCAGAGCGCACATCGCCCACAGTCCCCGAGAAGTTGGGGGGAGGGGTCGGCAAT
TGAACCGGTGCCTAGAGAAGGTGGCGCGGGGTAAACTGGGAAAGTGATGTCGTGTA
CTGGCTCCGCCTTTTTCCCGAGGGTGGGGGAGAACCGTATATAAGTGCAGTAGTCGC
CGTGAACGTTCTTTTTCGCAACGGGTTTGCCGCCAGAACACAGGTGTCGTGACGCGG
GATCCGCCACCATGCTTCTCCTGGTGACAAGCCTTCTGCTCTGTGAGTTACCACACC
CAGCATTCCTCCTGATCCCAGACATCCAGATGACACAGACTACATCCTCCCTGTCTG
CCTCTCTGGGAGACAGAGTCACCATCAGTTGCAGGGCAAGTCAGGACATTAGTAAA
TATTTAAATTGGTATCAGCAGAAACCAGATGGAACTGTTAAACTCCTGATCTACCAT
ACATCAAGATTACACTCAGGAGTCCCATCAAGGTTCAGTGGCAGTGGGTCTGGAAC
AGATTATTCTCTCACCATTAGCAACCTGGAGCAAGAAGATATTGCCACTTACTTTTG
CCAACAGGGTAATACGCTTCCGTACACGTTCGGAGGGGGGACTAAGTTGGAAATAA
CAGGCTCCACCTCTGGATCCGGCAAGCCCGGATCTGGCGAGGGATCCACCAAGGGC
GAGGTGAAACTGCAGGAGTCAGGACCTGGCCTGGTGGCGCCCTCACAGAGCCTGTC
CGTCACATGCACTGTCTCAGGGGTCTCATTACCCGACTATGGTGTAAGCTGGATTCG
CCAGCCTCCACGAAAGGGTCTGGAGTGGCTGGGAGTAATATGGGGTAGTGAAACCA
CATACTATAATTCAGCTCTCAAATCCAGACTGACCATCATCAAGGACAACTCCAAGA
GCCAAGTTTTCTTAAAAATGAACAGTCTGCAAACTGATGACACAGCCATTTACTACT
GTGCCAAACATTATTACTACGGTGGTAGCTATGCTATGGACTACTGGGGTCAAGGAA
CCTCAGTCACCGTCTCCTCAGCGGCCGCAATTGAAGTTATGTATCCTCCTCCTTACCT
AGACAATGAGAAGAGCAATGGAACCATTATCCATGTGAAAGGGAAACACCTTTGTC
CAAGTCCCCTATTTCCCGGACCTTCTAAGCCCTTTTGGGTGCTGGTGGTGGTTGGGG
GAGTCCTGGCTTGCTATAGCTTGCTAGTAACAGTGGCCTTTATTATTTTCTGGGTGAG
GAGTAAGAGGAGCAGGCTCCTGCACAGTGACTACATGAACATGACTCCCCGCCGCC
CCGGGCCCACCCGCAAGCATTACCAGCCCTATGCCCCACCACGCGACTTCGCAGCCT
ATCGCTCCAGAGTGAAGTTCAGCAGGAGCGCAGACGCCCCCGCGTACCAGCAGGGC
CAGAACCAGCTCTATAACGAGCTCAATCTAGGACGAAGAGAGGAGTACGATGTTTT
GGACAAGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGCCTACAG
TGAGATTGGGATGAAAGGCGAGCGCCGGAGGGGCAAGGGGCACGATGGCCTTTACC
AGGGTCTCAGTACAGCCACCAAGGACACCTACGACGCCCTTCACATGCAGGCCCTG
CCCCCTCGCTAA-3′
3′-
CCCCCGTCTCGCGTGTAGCGGGTGTCAGGGGCTCTTCAACCCCCCTCCCCAGCCGTT
AACTTGGCCACGGATCTCTTCCACCGCGCCCCATTTGACCCTTTCACTACAGCACAT
GACCGAGGCGGAAAAAGGGCTCCCACCCCCTCTTGGCATATATTCACGTCATCAGC
GGCACTTGCAAGAAAAAGCGTTGCCCAAACGGCGGTCTTGTGTCCACAGCACTGCG
CCCTAGGCGGTGGTACGAAGAGGACCACTGTTCGGAAGACGAGACACTCAATGGTG
TGGGTCGTAAGGAGGACTAGGGTCTGTAGGTCTACTGTGTCTGATGTAGGAGGGAC
AGACGGAGAGACCCTCTGTCTCAGTGGTAGTCAACGTCCCGTTCAGTCCTGTAATCA
TTTATAAATTTAACCATAGTCGTCTTTGGTCTACCTTGACAATTTGAGGACTAGATGG
TATGTAGTTCTAATGTGAGTCCTCAGGGTAGTTCCAAGTCACCGTCACCCAGACCTT
GTCTAATAAGAGAGTGGTAATCGTTGGACCTCGTTCTTCTATAACGGTGAATGAAAA
CGGTTGTCCCATTATGCGAAGGCATGTGCAAGCCTCCCCCCTGATTCAACCTTTATTG
TCCGAGGTGGAGACCTAGGCCGTTCGGGCCTAGACCGCTCCCTAGGTGGTTCCCGCT
CCACTTTGACGTCCTCAGTCCTGGACCGGACCACCGCGGGAGTGTCTCGGACAGGCA
GTGTACGTGACAGAGTCCCCAGAGTAATGGGCTGATACCACATTCGACCTAAGCGG
TCGGAGGTGCTTTCCCAGACCTCACCGACCCTCATTATACCCCATCACTTTGGTGTAT
GATATTAAGTCGAGAGTTTAGGTCTGACTGGTAGTAGTTCCTGTTGAGGTTCTCGGT
TCAAAAGAATTTTTACTTGTCAGACGTTTGACTACTGTGTCGGTAAATGATGACACG
GTTTGTAATAATGATGCCACCATCGATACGATACCTGATGACCCCAGTTCCTTGGAG
TCAGTGGCAGAGGAGTCGCCGGCGTTAACTTCAATACATAGGAGGAGGAATGGATC
TGTTACTCTTCTCGTTACCTTGGTAATAGGTACACTTTCCCTTTGTGGAAACAGGTTC
AGGGGATAAAGGGCCTGGAAGATTCGGGAAAACCCACGACCACCACCAACCCCCTC
AGGACCGAACGATATCGAACGATCATTGTCACCGGAAATAATAAAAGACCCACTCC
TCATTCTCCTCGTCCGAGGACGTGTCACTGATGTACTTGTACTGAGGGGCGGCGGGG
CCCGGGTGGGCGTTCGTAATGGTCGGGATACGGGGTGGTGCGCTGAAGCGTCGGAT
AGCGAGGTCTCACTTCAAGTCGTCCTCGCGTCTGCGGGGGCGCATGGTCGTCCCGGT
CTTGGTCGAGATATTGCTCGAGTTAGATCCTGCTTCTCTCCTCATGCTACAAAACCTG
TTCTCTGCACCGGCCCTGGGACTCTACCCCCCTTTCGGCTCTTCCTTCTTGGGAGTCC
TTCCGGACATGTTACTTGACGTCTTTCTATTCTACCGCCTCCGGATGTCACTCTAACC
CTACTTTCCGCTCGCGGCCTCCCCGTTCCCCGTGCTACCGGAAATGGTCCCAGAGTC
ATGTCGGTGGTTCCTGTGGATGCTGCGGGAAGTGTACGTCCGGGACGGGGGAGCGA
TT-5′
LENGTH: 1707
TYPE: DOUBLE-STRAND DNA
ORGANISM: ARTIFICAL
FEATURE: INSERTS A CHIMERIC ANTIGEN RECEPTOR TARGTING CD19 INTO THE
AAVSI SITE; DNA ORIENTATION IS INDICATED BY 5′ or 3′; 2 NUCLEOTIDE
OVERHANG INDICATED BY UNDERLINE
OTHER INFORMATION:
SEQ ID NO 24
5′-
TCTTAAGATCTTTGCTTTCCCTTCTCTATAACTTCGTATAGCATACATTATACGAAGT
TATATTCGTCT-3′
3′-
CCAGAATTCTAGAAACGAAAGGGAAGAGATATTGAAGCATATCGTATGTAATATGC
TTCAATATAAGCAGA-5′
LENGTH: 71
TYPE: DOUBLE-STRAND DNA
ORGANISM: ARTIFICAL
FEATURE: INSERTS A LOXP CASSETTE INTO THE AAVSI SITE; DNA ORIENTATION
IS INDICATED BY 5′ or 3′
OTHER INFORMATION: 2 NUCLEOTIDE OVERHANG INDICATED BY UNDERLINE
SEQ ID NO 25
MKRNYILGLEIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEEASKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,053
TYPE: AMINO ACID
ORGANISM: ARTIFICAL
FEATURE: SACAS9 D10E + N580A MUTANT
OTHER INFORMATION: Mutation underlined
SEQ ID NO 26
MKRNYILGLAIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEEASKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,053
TYPE: AMINO ACID
ORGANISM: ARTIFICAL
FEATURE: SACAS9 D10A + N580A MUTANT
OTHER INFORMATION: Mutation underlined
SEQ ID NO 27
MDKKYSIGLEIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDA
ILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFESPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENII
HLFTLTNLGAPAAFKYFDTTIDRKQYRSTKEVLDATLIHQSITGLYETRIDLSQLGGD
LENGTH: 1,368
TYPE: AMINO ACID
ORGANISM: ARTIFICAL
FEATURE: SPCAS9 D10E, D1135E, R1335Q, T1337R MUTANT
OTHER INFORMATION: mutations underlined
SEQ ID NO 28
MDKKYSIGLEIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDA
ILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDAIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFESPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENII
HLFTLTNLGAPAAFKYFDTTIDRKQYRSTKEVLDATLIHQSITGLYETRIDLSQLGGD
LENGTH: 1,368
TYPE: AMINO ACID
ORGANISM: ARTIFICAL
FEATURE: SPCAS9 D10E, H840A, D1135E, R1335Q, T1337R MUTANT
OTHER INFORMATION: mutations underlined
SEQ ID NO: 29
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGTGGS
GKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[WT]-saCas9[WT]
OTHER INFORMATION:
SEQ ID NO: 30
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETFKAK
MLKLGPDGRKALYSKPGSKNGRWNPETHKFCKCGVRIQTSAYTCSKCRNGGSGGTGGS
GKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[V117F]-saCas9[WT]
OTHER INFORMATION: Mutations underlined
SEQ ID NO: 31
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSRPGSKSGRWNPETHKFCKCGVRIQTSAYTCSKCRNGGSGGTGGS
GKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[K135R/N140S]-saCas9[WT]
OTHER INFORMATION: Mutations underlined
SEQ ID NO: 32
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETFKAK
MLKLGPDGRKALYSRPGSKSGRWNPETHKFCKCGVRIQTSAYTCSKCRNGGSGGTGGS
GKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[V117F/K135R/N140S]-saCas9[WT]
OTHER INFORMATION: Mutations underlined
SEQ ID NO: 33
MGKSGIYQIKNTLNNKVYVGSAKDFERRWKRHFKDLEKGCHSSIKLQRSFNKHGNVFE
CSILEEIPYEKDLIIERENFWIKELNSKINGYNIADASFGDTCSTHPLKEEIIKKRSETVKAK
MLKLGPDGRKALYSKPGSKNGRWNPETHKFCKCGVRIRTSAYTCSKCRNGGSGGTGGS
GKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[K26R/T95S/Q158R]-saCas9[WT]
OTHER INFORMATION: Mutations underlined
SEQ ID NO: 34
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGTGGS
GKRNYILGLEIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[WT]-saCas9[D10E]
OTHER INFORMATION: Mutations underlined
SEQ ID NO: 35
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETFKAK
MLKLGPDGRKALYSKPGSKNGRWNPETHKFCKCGVRIQTSAYTCSKCRNGGSGGTGGS
GKRNYILGLEIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[V117F]-saCas9[D10E]
OTHER INFORMATION: Mutations underlined
SEQ ID NO: 36
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSRPGSKSGRWNPETHKFCKCGVRIQTSAYTCSKCRNGGSGGTGGS
GKRNYILGLEIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[K135R/N140S]-saCas9[D10E]
OTHER INFORMATION: Mutations underlined
SEQ ID NO: 37
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETFKAK
MLKLGPDGRKALYSRPGSKSGRWNPETHKFCKCGVRIQTSAYTCSKCRNGGSGGTGGS
GKRNYILGLEIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[V117F/K135R/N140S]-saCas9[D10E]
OTHER INFORMATION: Mutations underlined
SEQ ID NO: 38
MGKSGIYQIKNTLNNKVYVGSAKDFERRWKRHFKDLEKGCHSSIKLQRSFNKHGNVFE
CSILEEIPYEKDLIIERENFWIKELNSKINGYNIADASFGDTCSTHPLKEEIIKKRSETVKAK
MLKLGPDGRKALYSKPGSKNGRWNPETHKFCKCGVRIRTSAYTCSKCRNGGSGGTGGS
GKRNYILGLEIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[K26R/T95S/Q158R]-saCas9[D10E]
OTHER INFORMATION: Mutations underlined
SEQ ID NO: 39
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGTGGS
GKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDAIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[WT]-saCas9[H557A]
OTHER INFORMATION: Mutations underlined
SEQ ID NO: 40
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETFKAK
MLKLGPDGRKALYSKPGSKNGRWNPETHKFCKCGVRIQTSAYTCSKCRNGGSGGTGGS
GKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDAIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[V117F]-saCas9[H557A]
OTHER INFORMATION: Mutations underlined
SEQ ID NO: 41
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSRPGSKSGRWNPETHKFCKCGVRIQTSAYTCSKCRNGGSGGTGGS
GKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDAIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[K135R/N140S]-saCas9[H557A]
OTHER INFORMATION: Mutations underlined
SEQ ID NO: 42
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETFKAK
MLKLGPDGRKALYSRPGSKSGRWNPETHKFCKCGVRIQTSAYTCSKCRNGGSGGTGGS
GKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDAIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[V117F/K135R/N140S]-saCas9[H557A]
OTHER INFORMATION: Mutations underlined
SEQ ID NO: 43
MGKSGIYQIKNTLNNKVYVGSAKDFERRWKRHFKDLEKGCHSSIKLQRSFNKHGNVFE
CSILEEIPYEKDLIIERENFWIKELNSKINGYNIADASFGDTCSTHPLKEEIIKKRSETVKAK
MLKLGPDGRKALYSKPGSKNGRWNPETHKFCKCGVRIRTSAYTCSKCRNGGSGGTGGS
GKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDAIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,232
TYPE: AMINO ACID
ORGANISM: ARTIFICAL
FEATURE: SYNTHETIC POLYPEPTIDE; Tev[K26R/T95S/Q158R]-saCas9[H557A]
OTHER INFORMATION: Mutations underlined
SEQ ID NO: 44
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGTGGS
GTQFEGFTNLYQVSKTLRFELIPQGKTLKHIQEQGFIEEDKARNDHYKELKPIIDRIYKTY
ADQCLQLVQLDWENLSAAIDSYRKEKTEETRNALIEEQATYRNAIHDYFIGRTDNLTDAI
NKRHAEIYKGLFKAELFNGKVLKQLGTVTTTEHENALLRSFDKFTTYFSGFYENRKNVF
SAEDISTAIPHRIVQDNFPKFKENCHIFTRLITAVPSLREHFENVKKAIGIFVSTSIEEVFSFP
FYNQLLTQTQIDLYNQLLGGISREAGTEKIKGLNEVLNLAIQKNDETAHIIASLPHRFIPLF
KQILSDRNTLSFILEEFKSDEEVIQSFCKYKTLLRNENVLETAEALFNELNSIDLTHIFISHK
KLETISSALCDHWDTLRNALYERRISELTGKITKSAKEKVQRSLKHEDINLQEIISAAGKE
LSEAFKQKTSEILSHAHAALDQPLPTTLKKQEEKEILKSQLDSLLGLYHLLDWFAVDESN
EVDPEFSARLTGIKLEMEPSLSFYNKARNYATKKPYSVEKFKLNFQMPTLASGWDVNKE
KNNGAILFVKNGLYYLGIMPKQKGRYKALSFEPTEKTSEGFDKMYYDYFPDAAKMIPK
CSTQLKAVTAHFQTHTTPILLSNNFIEPLEITKEIYDLNNPEKEPKKFQTAYAKKTGDQKG
YREALCKWIDFTRDFLSKYTKTTSIDLSSLRPSSQYKDLGEYYAELNPLLYHISFQRIAEK
EIMDAVETGKLYLFQIYNKDFAKGHHGKPNLHTLYWTGLFSPENLAKTSIKLNGQAELF
YRPKSRMKRMAHRLGEKMLNKKLKDQKTPIPDTLYQELYDYVNHRLSHDLSDEARAL
LPNVITKEVSHEIIKDRRFTSDKFFFHVPITLNYQAANSPSKFNQRVNAYLKEHPETPIIGI
DRGERNLIYITVIDSTGKILEQRSLNTIQQFDYQKKLDNREKERVAARQAWSVVGTIKDL
KQGYLSQVIHEIVDLMIHYQAVVVLENLNFGFKSKRTGIAEKAVYQQFEKMLIDKLNCL
VLKDYPAEKVGGVLNPYQLTDQFTSFAKMGTQSGFLFYVPAPYTSKIDPLTGFVDPFVW
KTIKNHESRKHFLEGFDFLHYDVKTGDFILHFKMNRNLSFQRGLPGFMPAWDIVFEKNE
TQFDAKGTPFIAGKRIVPVIENHRFTGRYRDLYPANELIALLEEKGIVERDGSNILPKLLEN
DDSHAIDTMVALIRSVLQMRNSNAATGEDYINSPVRDLNGVCFDSRFQNPEWPMDADA
NGAYHIALKGQLLLNHLKESKDLKLQNGISNQDWLAYIQELRN
LENGTH: 1,486
TYPE: AMINO ACID
ORGANISM: ARTIFICAL
FEATURE: SYNTHETIC POLYPEPTIDE; Tev[WT]-Cas12a[WT]
OTHER INFORMATION:
SEQ ID NO: 45
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETFKAK
MLKLGPDGRKALYSKPGSKNGRWNPETHKFCKCGVRIQTSAYTCSKCRNGGSGGTGGS
GTQFEGFTNLYQVSKTLRFELIPQGKTLKHIQEQGFIEEDKARNDHYKELKPIIDRIYKTY
ADQCLQLVQLDWENLSAAIDSYRKEKTEETRNALIEEQATYRNAIHDYFIGRTDNLTDAI
NKRHAEIYKGLFKAELFNGKVLKQLGTVTTTEHENALLRSFDKFTTYFSGFYENRKNVF
SAEDISTAIPHRIVQDNFPKFKENCHIFTRLITAVPSLREHFENVKKAIGIFVSTSIEEVFSFP
FYNQLLTQTQIDLYNQLLGGISREAGTEKIKGLNEVLNLAIQKNDETAHIIASLPHRFIPLF
KQILSDRNTLSFILEEFKSDEEVIQSFCKYKTLLRNENVLETAEALFNELNSIDLTHIFISHK
KLETISSALCDHWDTLRNALYERRISELTGKITKSAKEKVQRSLKHEDINLQEIISAAGKE
LSEAFKQKTSEILSHAHAALDQPLPTTLKKQEEKEILKSQLDSLLGLYHLLDWFAVDESN
EVDPEFSARLTGIKLEMEPSLSFYNKARNYATKKPYSVEKFKLNFQMPTLASGWDVNKE
KNNGAILFVKNGLYYLGIMPKQKGRYKALSFEPTEKTSEGFDKMYYDYFPDAAKMIPK
CSTQLKAVTAHFQTHTTPILLSNNFIEPLEITKEIYDLNNPEKEPKKFQTAYAKKTGDQKG
YREALCKWIDFTRDFLSKYTKTTSIDLSSLRPSSQYKDLGEYYAELNPLLYHISFQRIAEK
EIMDAVETGKLYLFQIYNKDFAKGHHGKPNLHTLYWTGLFSPENLAKTSIKLNGQAELF
YRPKSRMKRMAHRLGEKMLNKKLKDQKTPIPDTLYQELYDYVNHRLSHDLSDEARAL
LPNVITKEVSHEIIKDRRFTSDKFFFHVPITLNYQAANSPSKFNQRVNAYLKEHPETPIIGI
DRGERNLIYITVIDSTGKILEQRSLNTIQQFDYQKKLDNREKERVAARQAWSVVGTIKDL
KQGYLSQVIHEIVDLMIHYQAVVVLENLNFGFKSKRTGIAEKAVYQQFEKMLIDKLNCL
VLKDYPAEKVGGVLNPYQLTDQFTSFAKMGTQSGFLFYVPAPYTSKIDPLTGFVDPFVW
KTIKNHESRKHFLEGFDFLHYDVKTGDFILHFKMNRNLSFQRGLPGFMPAWDIVFEKNE
TQFDAKGTPFIAGKRIVPVIENHRFTGRYRDLYPANELIALLEEKGIVFRDGSNILPKLLEN
DDSHAIDTMVALIRSVLQMRNSNAATGEDYINSPVRDLNGVCFDSRFQNPEWPMDADA
NGAYHIALKGQLLLNHLKESKDLKLQNGISNQDWLAYIQELRN
LENGTH: 1,486
TYPE: AMINO ACID
ORGANISM: ARTIFICAL
FEATURE: SYNTHETIC POLYPEPTIDE; Tev[V117F]-Cas12a[WT]
OTHER INFORMATION: Mutations underlined
SEQ ID NO: 46
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSRPGSKSGRWNPETHKFCKCGVRIQTSAYTCSKCRNGGSGGTGGS
GTQFEGFTNLYQVSKTLRFELIPQGKTLKHIQEQGFIEEDKARNDHYKELKPIIDRIYKTY
ADQCLQLVQLDWENLSAAIDSYRKEKTEETRNALIEEQATYRNAIHDYFIGRTDNLTDAI
NKRHAEIYKGLFKAELFNGKVLKQLGTVTTTEHENALLRSFDKFTTYFSGFYENRKNVF
SAEDISTAIPHRIVQDNFPKFKENCHIFTRLITAVPSLREHFENVKKAIGIFVSTSIEEVFSFP
FYNQLLTQTQIDLYNQLLGGISREAGTEKIKGLNEVLNLAIQKNDETAHIIASLPHRFIPLF
KQILSDRNTLSFILEEFKSDEEVIQSFCKYKTLLRNENVLETAEALFNELNSIDLTHIFISHK
KLETISSALCDHWDTLRNALYERRISELTGKITKSAKEKVQRSLKHEDINLQEIISAAGKE
LSEAFKQKTSEILSHAHAALDQPLPTTLKKQEEKEILKSQLDSLLGLYHLLDWFAVDESN
EVDPEFSARLTGIKLEMEPSLSFYNKARNYATKKPYSVEKFKLNFQMPTLASGWDVNKE
KNNGAILFVKNGLYYLGIMPKQKGRYKALSFEPTEKTSEGFDKMYYDYFPDAAKMIPK
CSTQLKAVTAHFQTHTTPILLSNNFIEPLEITKEIYDLNNPEKEPKKFQTAYAKKTGDQKG
YREALCKWIDFTRDFLSKYTKTTSIDLSSLRPSSQYKDLGEYYAELNPLLYHISFQRIAEK
EIMDAVETGKLYLFQIYNKDFAKGHHGKPNLHTLYWTGLFSPENLAKTSIKLNGQAELF
YRPKSRMKRMAHRLGEKMLNKKLKDQKTPIPDTLYQELYDYVNHRLSHDLSDEARAL
LPNVITKEVSHEIIKDRRFTSDKFFFHVPITLNYQAANSPSKFNQRVNAYLKEHPETPIIGI
DRGERNLIYITVIDSTGKILEQRSLNTIQQFDYQKKLDNREKERVAARQAWSVVGTIKDL
KQGYLSQVIHEIVDLMIHYQAVVVLENLNFGFKSKRTGIAEKAVYQQFEKMLIDKLNCL
VLKDYPAEKVGGVLNPYQLTDQFTSFAKMGTQSGFLFYVPAPYTSKIDPLTGFVDPFVW
KTIKNHESRKHFLEGFDFLHYDVKTGDFILHFKMNRNLSFQRGLPGFMPAWDIVFEKNE
TQFDAKGTPFIAGKRIVPVIENHRFTGRYRDLYPANELIALLEEKGIVERDGSNILPKLLEN
DDSHAIDTMVALIRSVLQMRNSNAATGEDYINSPVRDLNGVCFDSRFQNPEWPMDADA
NGAYHIALKGQLLLNHLKESKDLKLQNGISNQDWLAYIQELRN
LENGTH: 1,486
TYPE: AMINO ACID
ORGANISM: ARTIFICAL
FEATURE: SYNTHETIC POLYPEPTIDE; Tev[K135R/N140S]-Cas12a[WT]
OTHER INFORMATION: Mutations underlined
SEQ ID NO: 47
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETFKAK
MLKLGPDGRKALYSRPGSKSGRWNPETHKFCKCGVRIQTSAYTCSKCRNGGSGGTGGS
GTQFEGFTNLYQVSKTLRFELIPQGKTLKHIQEQGFIEEDKARNDHYKELKPIIDRIYKTY
ADQCLQLVQLDWENLSAAIDSYRKEKTEETRNALIEEQATYRNAIHDYFIGRTDNLTDAI
NKRHAEIYKGLFKAELFNGKVLKQLGTVTTTEHENALLRSFDKFTTYFSGFYENRKNVF
SAEDISTAIPHRIVQDNFPKFKENCHIFTRLITAVPSLREHFENVKKAIGIFVSTSIEEVFSFP
FYNQLLTQTQIDLYNQLLGGISREAGTEKIKGLNEVLNLAIQKNDETAHIIASLPHRFIPLF
KQILSDRNTLSFILEEFKSDEEVIQSFCKYKTLLRNENVLETAEALFNELNSIDLTHIFISHK
KLETISSALCDHWDTLRNALYERRISELTGKITKSAKEKVQRSLKHEDINLQEIISAAGKE
LSEAFKQKTSEILSHAHAALDQPLPTTLKKQEEKEILKSQLDSLLGLYHLLDWFAVDESN
EVDPEFSARLTGIKLEMEPSLSFYNKARNYATKKPYSVEKFKLNFQMPTLASGWDVNKE
KNNGAILFVKNGLYYLGIMPKQKGRYKALSFEPTEKTSEGFDKMYYDYFPDAAKMIPK
CSTQLKAVTAHFQTHTTPILLSNNFIEPLEITKEIYDLNNPEKEPKKFQTAYAKKTGDQKG
YREALCKWIDFTRDFLSKYTKTTSIDLSSLRPSSQYKDLGEYYAELNPLLYHISFQRIAEK
EIMDAVETGKLYLFQIYNKDFAKGHHGKPNLHTLYWTGLFSPENLAKTSIKLNGQAELF
YRPKSRMKRMAHRLGEKMLNKKLKDQKTPIPDTLYQELYDYVNHRLSHDLSDEARAL
LPNVITKEVSHEIIKDRRFTSDKFFFHVPITLNYQAANSPSKFNQRVNAYLKEHPETPIIGI
DRGERNLIYITVIDSTGKILEQRSLNTIQQFDYQKKLDNREKERVAARQAWSVVGTIKDL
KQGYLSQVIHEIVDLMIHYQAVVVLENLNFGFKSKRTGIAEKAVYQQFEKMLIDKLNCL
VLKDYPAEKVGGVLNPYQLTDQFTSFAKMGTQSGFLFYVPAPYTSKIDPLTGFVDPFVW
KTIKNHESRKHFLEGFDFLHYDVKTGDFILHFKMNRNLSFQRGLPGFMPAWDIVFEKNE
TQFDAKGTPFIAGKRIVPVIENHRFTGRYRDLYPANELIALLEEKGIVERDGSNILPKLLEN
DDSHAIDTMVALIRSVLQMRNSNAATGEDYINSPVRDLNGVCFDSRFQNPEWPMDADA
NGAYHIALKGQLLLNHLKESKDLKLQNGISNQDWLAYIQELRN
LENGTH: 1,486
TYPE: AMINO ACID
ORGANISM: ARTIFICAL
FEATURE: SYNTHETIC POLYPEPTIDE; Tev[V117F/K135R/N140S]-Cas12a[WT]
OTHER INFORMATION: Mutations underlined
SEQ ID NO: 48
MGKSGIYQIKNTLNNKVYVGSAKDFERRWKRHFKDLEKGCHSSIKLQRSFNKHGNVFE
CSILEEIPYEKDLIIERENFWIKELNSKINGYNIADASFGDTCSTHPLKEEIIKKRSETVKAK
MLKLGPDGRKALYSKPGSKNGRWNPETHKFCKCGVRIRTSAYTCSKCRNGGSGGTGGS
GTQFEGFTNLYQVSKTLRFELIPQGKTLKHIQEQGFIEEDKARNDHYKELKPIIDRIYKTY
ADQCLQLVQLDWENLSAAIDSYRKEKTEETRNALIEEQATYRNAIHDYFIGRTDNLTDAI
NKRHAEIYKGLFKAELFNGKVLKQLGTVTTTEHENALLRSFDKFTTYFSGFYENRKNVF
SAEDISTAIPHRIVQDNFPKFKENCHIFTRLITAVPSLREHFENVKKAIGIFVSTSIEEVFSFP
FYNQLLTQTQIDLYNQLLGGISREAGTEKIKGLNEVLNLAIQKNDETAHIIASLPHRFIPLF
KQILSDRNTLSFILEEFKSDEEVIQSFCKYKTLLRNENVLETAEALFNELNSIDLTHIFISHK
KLETISSALCDHWDTLRNALYERRISELTGKITKSAKEKVQRSLKHEDINLQEIISAAGKE
LSEAFKQKTSEILSHAHAALDQPLPTTLKKQEEKEILKSQLDSLLGLYHLLDWFAVDESN
EVDPEFSARLTGIKLEMEPSLSFYNKARNYATKKPYSVEKFKLNFQMPTLASGWDVNKE
KNNGAILFVKNGLYYLGIMPKQKGRYKALSFEPTEKTSEGFDKMYYDYFPDAAKMIPK
CSTQLKAVTAHFQTHTTPILLSNNFIEPLEITKEIYDLNNPEKEPKKFQTAYAKKTGDQKG
YREALCKWIDFTRDFLSKYTKTTSIDLSSLRPSSQYKDLGEYYAELNPLLYHISFQRIAEK
EIMDAVETGKLYLFQIYNKDFAKGHHGKPNLHTLYWTGLFSPENLAKTSIKLNGQAELF
YRPKSRMKRMAHRLGEKMLNKKLKDQKTPIPDTLYQELYDYVNHRLSHDLSDEARAL
LPNVITKEVSHEIIKDRRFTSDKFFFHVPITLNYQAANSPSKFNQRVNAYLKEHPETPIIGI
DRGERNLIYITVIDSTGKILEQRSLNTIQQFDYQKKLDNREKERVAARQAWSVVGTIKDL
KQGYLSQVIHEIVDLMIHYQAVVVLENLNFGFKSKRTGIAEKAVYQQFEKMLIDKLNCL
VLKDYPAEKVGGVLNPYQLTDQFTSFAKMGTQSGFLFYVPAPYTSKIDPLTGFVDPFVW
KTIKNHESRKHFLEGFDFLHYDVKTGDFILHFKMNRNLSFQRGLPGFMPAWDIVFEKNE
TQFDAKGTPFIAGKRIVPVIENHRFTGRYRDLYPANELIALLEEKGIVFRDGSNILPKLLEN
DDSHAIDTMVALIRSVLQMRNSNAATGEDYINSPVRDLNGVCFDSRFQNPEWPMDADA
NGAYHIALKGQLLLNHLKESKDLKLQNGISNQDWLAYIQELRN
LENGTH: 1,486
TYPE: AMINO ACID
ORGANISM: ARTIFICAL
FEATURE: SYNTHETIC POLYPEPTIDE; Tev[K26R/T95S/Q158R]-Cas12a[WT]
OTHER INFORMATION: Mutations underlined
SEQ ID NO: 49
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGTGGS
QEIKRINKIRRRLVKDSNTKKAGKTGPMKTLLVRVMTPDLRERLENLRKKPENIPQPISN
TSRANLNKLLTDYTEMKKAILHVYWEEFQKDPVGLMSRVAQPAPKNIDQRKLIPVKDG
NERLTSSGFACSQCCQPLYVYKLEQVNDKGKPHTNYFGRCNVSEHERLILLSPHKPEAN
DELVTYSLGKFGQRALDFYSIHVTRESNHPVKPLEQIGGNSCASGPVGKALSDACMGAV
ASFLTKYQDIILEHQKVIKKNEKRLANLKDIASANGLAFPKITLPPQPHTKEGIEAYNNVV
AQIVIWVNLNLWQKLKIGRDEAKPLQRLKGFPSFPLVERQANEVDWWDMVCNVKKLI
NEKKEDGKVFWQNLAGYKRQEALLPYLSSEEDRKKGKKFARYQFGDLLLHLEKKHGE
DWGKVYDEAWERIDKKVEGLSKHIKLEEERRSEDAQSKAALTDWLRAKASFVIEGLKE
ADKDEFCRCELKLOKWYGDLRGKPFAIEAENSILDISGFSKQYNCAFIWQKDGVKKLNL
YLIINYFKGGKLRFKKIKPEAFEANRFYTVINKKSGEIVPMEVNFNFDDPNLIILPLAFGKR
QGREFIWNDLLSLETGSLKLANGRVIEKTLYNRRTRQDEPALFVALTFERREVLDSSNIK
PMNLIGIDRGENIPAVIALTDPEGCPLSRFKDSLGNPTHILRIGESYKEKQRTIQAAKEVEQ
RRAGGYSRKYASKAKNLADDMVRNTARDLLYYAVTQDAMLIFENLSRGFGRQGKRTF
MAERQYTRMEDWLTAKLAYEGLPSKTYLSKTLAQYTSKTCSNCGFTITSADYDRVLEK
LKKTATGWMTTINGKELKVEGQITYYNRYKRQNVVKDLSVELDRLSEESVNNDISSWT
KGRSGEALSLLKKRFSHRPVQEKFVCLNCGFETHADEQAALNIARSWLFLRSQEYKKYQ
TNKTTGNTDKRAFVETWQSFYRKKLKEVWKPAV
LENGTH: 1,156
TYPE: AMINO ACID
ORGANISM: ARTIFICAL
FEATURE: SYNTHETIC POLYPEPTIDE; Tev[WT]-CasX
OTHER INFORMATION:
SEQ ID NO 50
UAAUUUCUACUCUUGUAGAU
LENGTH: 20
TYPE: RNA
ORGANISM: ARTIFICAL
FEATURE: TARGETS AAVS1 GENE
OTHER INFORMATION:
SEQ ID 51 [Cas12a]
TQFEGFTNLYQVSKTLRFELIPQGKTLKHIQEQGFIEEDKARNDHYKELKPIIDRIYKTYA
DQCLQLVQLDWENLSAAIDSYRKEKTEETRNALIEEQATYRNAIHDYFIGRTDNLTDAIN
KRHAEIYKGLFKAELFNGKVLKQLGTVTTTEHENALLRSFDKFTTYFSGFYENRKNVFS
AEDISTAIPHRIVQDNFPKFKENCHIFTRLITAVPSLREHFENVKKAIGIFVSTSIEEVFSFPF
YNQLLTQTQIDLYNQLLGGISREAGTEKIKGLNEVLNLAIQKNDETAHIIASLPHRFIPLFK
QILSDRNTLSFILEEFKSDEEVIQSFCKYKTLLRNENVLETAEALFNELNSIDLTHIFISHKK
LETISSALCDHWDTLRNALYERRISELTGKITKSAKEKVQRSLKHEDINLQEIISAAGKEL
SEAFKQKTSEILSHAHAALDQPLPTTLKKQEEKEILKSQLDSLLGLYHLLDWFAVDESNE
VDPEFSARLTGIKLEMEPSLSFYNKARNYATKKPYSVEKFKLNFQMPTLASGWDVNKE
KNNGAILFVKNGLYYLGIMPKQKGRYKALSFEPTEKTSEGFDKMYYDYFPDAAKMIPK
CSTQLKAVTAHFQTHTTPILLSNNFIEPLEITKEIYDLNNPEKEPKKFQTAYAKKTGDQKG
YREALCKWIDFTRDFLSKYTKTTSIDLSSLRPSSQYKDLGEYYAELNPLLYHISFQRIAEK
EIMDAVETGKLYLFQIYNKDFAKGHHGKPNLHTLYWTGLFSPENLAKTSIKLNGQAELF
YRPKSRMKRMAHRLGEKMLNKKLKDQKTPIPDTLYQELYDYVNHRLSHDLSDEARAL
LPNVITKEVSHEIIKDRRFTSDKFFFHVPITLNYQAANSPSKFNQRVNAYLKEHPETPIIGI
DRGERNLIYITVIDSTGKILEQRSLNTIQQFDYQKKLDNREKERVAARQAWSVVGTIKDL
KQGYLSQVIHEIVDLMIHYQAVVVLENLNFGFKSKRTGIAEKAVYQQFEKMLIDKLNCL
VLKDYPAEKVGGVLNPYQLTDQFTSFAKMGTQSGFLFYVPAPYTSKIDPLTGFVDPFVW
KTIKNHESRKHFLEGFDFLHYDVKTGDFILHFKMNRNLSFQRGLPGFMPAWDIVFEKNE
TQFDAKGTPFIAGKRIVPVIENHRFTGRYRDLYPANELIALLEEKGIVFRDGSNILPKLLEN
DDSHAIDTMVALIRSVLQMRNSNAATGEDYINSPVRDLNGVCFDSRFQNPEWPMDADA
NGAYHIALKGQLLLNHLKESKDLKLQNGISNQDWLAYIQELRN
SEQ ID 52 [CasX]
QEIKRINKIRRRLVKDSNTKKAGKTGPMKTLLVRVMTPDLRERLENLRKKPENIPQPISN
TSRANLNKLLTDYTEMKKAILHVYWEEFQKDPVGLMSRVAQPAPKNIDQRKLIPVKDG
NERLTSSGFACSQCCQPLYVYKLEQVNDKGKPHTNYFGRCNVSEHERLILLSPHKPEAN
DELVTYSLGKFGQRALDFYSIHVTRESNHPVKPLEQIGGNSCASGPVGKALSDACMGAV
ASFLTKYQDIILEHQKVIKKNEKRLANLKDIASANGLAFPKITLPPQPHTKEGIEAYNNVV
AQIVIWVNLNLWQKLKIGRDEAKPLQRLKGFPSFPLVERQANEVDWWDMVCNVKKLI
NEKKEDGKVFWQNLAGYKRQEALLPYLSSEEDRKKGKKFARYQFGDLLLHLEKKHGE
DWGKVYDEAWERIDKKVEGLSKHIKLEEERRSEDAQSKAALTDWLRAKASFVIEGLKE
ADKDEFCRCELKLQKWYGDLRGKPFAIEAENSILDISGFSKQYNCAFIWQKDGVKKLNL
YLIINYFKGGKLRFKKIKPEAFEANRFYTVINKKSGEIVPMEVNFNFDDPNLIILPLAFGKR
QGREFIWNDLLSLETGSLKLANGRVIEKTLYNRRTRQDEPALFVALTFERREVLDSSNIK
PMNLIGIDRGENIPAVIALTDPEGCPLSRFKDSLGNPTHILRIGESYKEKQRTIQAAKEVEQ
RRAGGYSRKYASKAKNLADDMVRNTARDLLYYAVTQDAMLIFENLSRGFGRQGKRTF
MAERQYTRMEDWLTAKLAYEGLPSKTYLSKTLAQYTSKTCSNCGFTITSADYDRVLEK
LKKTATGWMTTINGKELKVEGQITYYNRYKRQNVVKDLSVELDRLSEESVNNDISSWT
KGRSGEALSLLKKRFSHRPVQEKFVCLNCGFETHADEQAALNIARSWLFLRSQEYKKYQ
TNKTTGNTDKRAFVETWQSFYRKKLKEVWKPAV
SEQ ID NO: 53 [I-TEVI WT NUCLEASE DOMAIN AND LINKER DOMAIN]
MKSGIYQIKNTLNNKVYVGSAKDFEKRWKRHFKDLEKGCHSSIKLQRSFNKHGNVFEC
SILEEIPYEKDLIIERENFWIKELNSKINGYNIADATFGDTCSTHPLKEEIIKKRSETVKAKM
LKLGPDGRKALYSKPGSKNGRWNPETHKFCKCGVRIQTSAYTCSKCRN
SEQ ID NO: 54 [CAS9 WT]
KRNYILGLDIGITSVGYGIIDYETRDVIDAGVRLFKEANVENNEGRRSKRGARRLKRRRR
HRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKE
AKQLLKVQKAYHQLDQSFIDTYIDLLETRRTYYEGPGEGSPFGWKDIKEWYEMLMGHC
TYFPEELRSVKYAYNADLYNALNDLNNLVITRDENEKLEYYEKFQIIENVFKQKKKPTL
KQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
SEQ ID NO: 55 [SV40 nuclear localization sequence]
PKKKRKV
SEQ ID NO: 56 [nucleoplasmin nuclear localization sequence]
KRPAATKKAGQAKKKK
SEQ ID NO: 57 [I-TevI linker fragment]
DATFGDTCSTHPLKEEIIKKRSETVKAKMLKLGPDGRKALYSKPGSKNGRWNPETHKFC
KCGVRIQTSAYTCSKCRN
SEQ ID 58 [I-TevI nuclease domain]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIA
SEQ ID 59 [I-TevI linker domain]
DATFGDTCSTHPLKEEIIKKRSETVKAKMLKLGPDGRKALYSKPGSKNGRWNPETHKFC
KCGVRIQTSAYTCSKCRNGGSGGS

1.-105. (canceled)

106. A chimeric nuclease comprising a modified I-TevI nuclease domain and a Cas9 domain, wherein the modified I-TevI nuclease domain comprises an amino acid sequence that is at least 90%, 95%, 97%, 98%, or 99% identical to SEQ ID NO: 8 or residues 1-92 of SEQ ID NO: 53.

107. The chimeric nuclease of claim 106, wherein the modified I-TevI nuclease domain comprises an amino acid modification at an amino acid residue corresponding to K26 of SEQ ID NO: 53, optionally wherein the modification is K26R.

108. The chimeric nuclease of claim 106, wherein the modified I-TevI nuclease domain comprises one or more amino acid substitutions selected from the group consisting of T11V, V16I, N14G, E25D, K26R, E36S, K37N, G38N, C39V, S41H, L45F, F49Y, I60V, and E81I of SEQ ID NO: 53.

109. The chimeric nuclease of claim 106, further comprising an I-TevI linker domain, wherein the I-TevI linker domain comprises residues 93-169 of SEQ ID NO: 53

110. The chimeric nuclease of claim 109, wherein the modified I-TevI nuclease domain and or the I-TevI linker domain comprises a modification at one or more amino acid residues corresponding to K26, T95, V117, K135, N140, and Q158 of SEQ ID NO: 53.

111. The chimeric nuclease of claim 109, wherein the I-TevI linker domain comprises one or more amino acid substitutions selected from the group consisting of T95S, S101Y, V117F, A119D, K120N, K135N, K135R, P126S, D127K, N140S, T147I, Q158R, A161V, and S165G.

112. The chimeric nuclease of claim 109, wherein
a. the modified I-TevI nuclease domain comprises residues 1-92 of SEQ ID NO: 53 and one or more amino acid substitutions selected from the group consisting of T11V, V16I, N14G, E25D, K26R, E36S, K37N, G38N, C39V, S41H, L45F, F49Y, I60V, and E81I; and
b. the I-TevI linker domain comprises residues 93-169 of SEQ ID NO: 53 and one or more amino acid substitutions selected from the group consisting of T95S, S101Y, V117F, A119D, K120N, K135N, K135R, P126S, D127K, N140S, T147I, Q158R, A161V and S165G.

113. The chimeric nuclease of claim 106, wherein the Cas9 domain comprises an amino acid sequence at least about 90%, 95%, 97%, 98%, or 99% identical, or is identical to SEQ ID NO: 15.

114. The chimeric nuclease of claim 113, wherein the Cas9 domain comprises SEQ ID NO: 15 having at least one amino acid substitution selected from the group consisting of D10A, D10E, H557A, N580A, H840A, D1135E, R1335Q, and T1337R.

115. The method of claim 106, wherein the Cas9 domain comprises a Streptococcus pyogenes Cas9 sequence having at least one substitution selected from the group consisting of D10A, D10E, D1135E, R1335Q, T1337R, and H840A.

116. The chimeric nuclease of claim 106, further comprising a guide RNA.

117. The chimeric nuclease of claim 116, wherein the guide RNA targets a genomic target in a mammalian cell.

118. The chimeric nuclease of claim 106, further comprising a nuclear localization signal.

119. The chimeric nuclease of claim 106, wherein the chimeric nuclease has cleavage activity against an I-TevI recognition site in double-stranded DNA.

120. A nucleic acid encoding the chimeric nuclease of claim 106.

121. A pharmaceutical composition comprising the chimeric nuclease of claim 106 and a pharmaceutically acceptable excipient, diluent, or carrier.

122. A composition comprising the chimeric nuclease of claim 106 encapsulated in a lipid nanoparticle.

123. A method of genetically engineering the genome of a cell, the method comprising administering the chimeric nuclease of claim 106 to the cell.

124. A chimeric nuclease comprising a modified I-TevI nuclease domain and a CasX domain, wherein the modified I-TevI nuclease domain comprises an amino acid sequence that is at least 90%, 95%, 97%, 98%, or 99% identical to SEQ ID NO: 8 or residues 1-92 of SEQ ID NO: 53.

125. A chimeric nuclease comprising a modified I-TevI nuclease domain and a Cas12 domain, wherein the modified I-TevI nuclease domain comprises an amino acid sequence that is at least 90%, 95%, 97%, 98%, or 99% identical to SEQ ID NO: 8 or residues 1-92 of SEQ ID NO: 53.
